(19) United States
(12) Patent Application Publication
(10) Pub. No.: US 2014/0221235 A1
(43) Pub. Date: Aug. 7, 2014

(54) BIOMARKER ALGORITHM FOR DETERMINING THE TIME OF STROKE SYMPTOM ONSET AND METHOD

(71) Applicant: Taura L. Barr, Waynesburg, PA (US)

(72) Inventor: Taura L. Barr, Waynesburg, PA (US)

(73) Assignee: WEST VIRGINIA UNIVERSITY, Morgantown, WV (US)

(21) Appl. No.: 14/167,059

(22) Filed: Jan. 29, 2014

Related U.S. Application Data
(60) Provisional application No. 61/759,657, filed on Feb. 1, 2013.

(51) Int. Cl. C12Q 1/68

(52) U.S. Cl. CPC C12Q 1/6883
USPC 506/9; 536/24.31; 530/389.2; 506/18; 506/16; 530/389.1

(57) ABSTRACT

A method of determining the time of stroke symptom onset is provided including obtaining a biological sample from an individual; contacting the biological sample with a detection composition comprising at least one expression mediator of a LY96, ARG1, CA4, and a TLR expression mediators, or a combination of these expression mediators, wherein at least one of the expression mediators is associated with an acute phase response of ischemic stroke, for forming a detectable response; and correlating the detectable response with a time of onset of one or more stroke symptoms. A composition is provided having a nucleic acid probe, an antibody, or a purified biomarker that is specific for at least one of a LY96, ARG1, CA4, and TLR expression mediators, or a combination of these expression mediators.

### CROSS-REFERENCE TO RELATED APPLICATIONS

This utility patent application claims the benefit of priority to pending U.S. Provisional Patent Application Ser. No. 61/759,657, filed on Feb. 1, 2013. The entire contents of U.S. Provisional Patent Application Ser. No. 61/759,657 is incorporated by reference into this utility patent application as if fully rewritten herein.

### STATEMENT REGARDING FEDERALLY SPONSORED RESEARCH OR DEVELOPMENT

Not applicable.

### SEQUENCE LISTING

Following the Abstract of the Disclosure is set forth a paper copy of the SEQUENCE LISTING in written form (.PDF format) having SEQ ID NO:1 through SEQ ID NO:8. The paper copy of the SEQUENCE LISTING is incorporated by reference into this application. A SEQUENCE LISTING in computer-readable form (.txt file) also accompanies this application with a Statement Of Identity Of Computer-Readable Form And Written Sequence Listing.

### BACKGROUND OF THE INVENTION

1. Field of the Invention

The present invention provides compositions for a diagnostic assay for the diagnosis of stroke symptom onset and a method of using these assays for determining the time of onset of a stroke in a patient. Moreover, the methods and compositions of the present invention can also be used to facilitate the treatment of stroke patients or other neurologic disease patients and the development of additional diagnostic and/or prognostic indicators. Specifically, the present invention relates to a method of determining the time of stroke symptom onset comprising obtaining a biological sample from an individual; contacting the biological sample with a detection composition comprising at least one or more of an expression mediator that is a Lymphocyte antigen 96 (LY96); a Arginase 1 (ARG1); a Carbonic anhydrase 4 (CA4); and/or a Toll-like receptors (TLR) expression mediator, or combinations thereof, and wherein at least one of these expression mediators is associated with an acute phase response of ischemic stroke, for forming a detectable response; and correlating the detectable response with a time of onset of one or more stroke symptoms.

2. Description of the Background Art

Stroke, also referred to as a cerebrovascular accident (CVA), is the rapid loss of brain function due to disturbance in the blood supply to the brain. There are two broad categories of stoke: ischemic stroke and hemorrhagic stroke. Ischemic stroke, also referred to as acute ischemic stroke (AIS), is usually caused by the interruption of blood supply, often by a thrombus (blood clot). Ischemic stroke can also be caused by a narrowing of a blood vessel(s) that supplies the brain. Ischemic stroke accounts for about 87% of strokes. In contrast, hemorrhagic stroke is caused by bleeding into the brain as a result from rupture of a blood vessel or an abnormal vascular structure. Intracerebral hemorrhages and subarachnoid hemorrhages make up 10% and 3% of strokes, respectively. Additionally, a patient may experience transient ischemic attacks, which is caused by the changes in the blood supply to a particular area of the brain. Transient ischemic attacks indicate a high risk for a future stroke and are defined as stroke symptoms that are resolved within 24 hours. In contrast, symptoms persisting longer than 24 hours are classified as stroke. However, recently the medical community has incorporated terms such as brain attach and acute ischemic cerebrovascular syndrome to distinguish stroke without the arbitrary time frame of 24 hours.

Ischemic stroke encompasses subtypes that at least include thrombotic, embolic, lacunar and hypoperfusion types of strokes. In a thrombotic stroke, blood flow is impaired due to the formation of a thrombus that causes blockage to one or more of the arteries supplying blood to the brain. In contrast, most embolic strokes occur when a thrombus forms in the body, usually the heart, and travels through the arterial bloodstream to the brain and to a blood vessel small enough to block passage of the thrombus. Embolic strokes can also be caused by substances other than a thrombus, including fat (atheroma), air, cancer cells, or bacteria. Lacunar, also referred to as small vessel disease, occurs when blood flow is blocked to small arterial vessels. Hypoperfusion is the reduction of blood flow to all parts of the body and is often caused by myocardial infarction, pulmonary embolism, pericardial effusion, or arrhythmias.

The symptoms of stroke often include sudden numbness or weakness, especially on one side of the body, often of the face, arm or leg; sudden confusion, trouble speaking or understanding; sudden trouble seeing in one or both eyes; sudden trouble walking, dizziness, loss of balance or coordination; and sudden severe headache with no known cause.

Stroke is currently ranked the fourth leading cause of death in the United States, ranking only behind heart disease, cancer, and chronic lower respiratory diseases. Approximately 795,000 strokes occur in the United States each year and cause 133,000 deaths each year. Further, there is an estimated 7 million stroke survivors in the United States over the age of 20 years old and acute ischemic stroke is the leading cause of long-term disability. The estimated cost of stroke in the United States is over $73 billion per year. As mentioned above, ischemic stroke accounts for 87% of instances of stroke, and consequently, the category of stroke contributing the greatest financial burden. Roger V L, Go A S, Lloyd-Jones D M, et al. Heart disease and stroke statistics-2011 update: a report from the American Heart Association. Circulation. 2011;123(4):e18-e209.

The risk of ischemic stroke is associated with a variety of controllable factors. These factors include hypertension (high blood pressure), atrial fibrillation, high cholesterol, diabetes, atherosclerosis, circulation problems, tobacco use, alcohol use, physical inactivity and obesity. Uncontrollable factors associated with the risk of ischemic stroke in a patient include age, race, gender, family history, fibromuscular dysplasia, and patent foramen ovale.

There is currently only one Food and Drug Administration (FDA) approved treatment for stroke. Tissue plasminogen activator (tPA), or recombinant tissue plasminogen activator (rtPA), has been the only FDA approved treatment for ischemic stroke since 1995. However, the powerful effects of tPA also come with significant clinical complications. Only 2-3% of all ischemic stroke patients receive tPA because of many contraindicating factors, the first primarily being when the patient arrives at the treatment facility compared to when their symptoms began. tPA is only FDA approved for up to 4.5 hours from onset of stroke symptoms. However, the median time patients arrive to the ED (emergency department) for treatment is around 8 hours. Increasing the time window for tPA treatment is a clinical need. In addition, up to 30% of patients are unaware of the time when their stroke symptoms began. In some cases, patients have gone to bed normal and then wake up in the morning with their symptoms. These patients cannot be given tPA because of the uncertainty surrounding the time when they were last known to be normal.

Prior to this invention, the determination of time of stroke symptom onset is often difficult and inaccurate, as discussed hereinabove, and especially when patients are severely comprised or the events are un-witnessed. These problems are due in part to limitations in the technology currently used to evaluate a patient for when their stroke began (clinician and patient/surrogate interaction) and limitations in the level of experience and/or proper training possessed by medical clinicians who engage the patients. These circumstances are detrimental to stroke and brain injury victims because accurate, nonbiased prediction of time of stroke onset is extremely important to the health and outcome of the patients at the point of care. The present invention is related to methods for determining the onset of stroke symptoms.

As mentioned hereinabove, tissue plasminogen activator (tPA) has been the only FDA approved treatment for ischemic stroke since 1995. The present invention discloses the strong innate inflammatory reaction to stroke and monitors the expression of these immune genes in the peripheral blood following stroke. The present invention discloses that the expression of these immune genes significantly decreases over time and thus can be used as a surrogate for when the stroke began. An unbiased measure of when stroke symptoms began would aid clinicians in their decision to treat with tPA. This could result in a 30% increase in utilization of tPA with an expected increase in functional recovery. These inflammatory immune markers may also be used to guide tPA treatment beyond the 4.5 hour time window. The methods of the present invention using these genomic biomarkers will guide stroke therapeutics.

The advancements of tPA therapy aside, there is still a demand for alternative acute ischemic stroke therapies in clinical practice. Unfortunately, the results of recent clinical trials have demonstrated that there is still a gap in the understanding of the variable human response to ischemic stroke. Numerous promising pre-clinical therapeutics display insignificant clinical utility in human patients, which speaks to the difficulty of translating what is learned at the bench to the patient at the bedside.

These negative findings may be due in part to the complexity of the human physiologic response to ischemic stroke, limited knowledge about the multiple pathways interacting in response to ischemic stroke and the implications of genomic variability on individual recovery from ischemic stroke. The difficulty may also be attributable to insufficient classification of ischemic stroke subtype. It is possible that gene expression profiling can help to identify subtypes of ischemic stroke, which has tremendous utility in designing therapeutic strategies for treatment. A better understanding of stroke pathophysiology in humans and more appropriate stroke subtyping may provide the foundation needed to design appropriate therapeutics for battling ischemic stroke and other stroke types. Because knowing the definitive time of onset is critical for treating stroke patients with tissue plasminogen activator (tPA) since treatment with tPA relies upon knowing the last known normal for administration of tPA within the 4.5 hour time window. However, the last known normals are often difficult to determine because of the un-witnessed stroke events, inability of the patient to communicate, or stroke symptoms are mild and not immediately noticed. Further, another limitation in the diagnosis of ischemic stroke is circumstances due to the rapid onset and progression of acute ischemic stroke, are such that ischemic stroke patients are often seen by clinicians not having the appropriate knowledge and training to be able to provide a correct, life-saving diagnosis. For example, brain imaging technology can be an important component in diagnosing an ischemic stroke. These technologies include, for example, brain computed tomography scan (brain CT scan), Magnetic Resonance Imaging (MRI), computed tomography arteriogram (CTA) and magnetic resonance arteriogram (MRA), carotid angiography, and carotid ultrasound. However, such technology is often not available and proper interpretation of brain imaging results concerning stroke diagnoses is best for highly and specifically trained clinicians. Therefore, achieving early and accurate diagnosis is often not possible due to current clinical circumstances.

Accordingly, there is a need for a rapid diagnostic test capable of making an unbiased and accurate clinical diagnosis of ischemic stroke. The present invention meets these unmet needs in the medical assessment of a stroke patient. The present invention provides a method for determining time from stroke symptom onset for use in the acute care clinical setting to improve utilization of the administration of tPA and streamline appropriate secondary prevention.

### BRIEF SUMMARY OF THE INVENTION

The present invention relates to the identification and use of diagnostic markers for the time of stroke onset. The present invention includes methods for rapid and early detection of stroke and a surrogate for when the stroke began to help facilitate medical treatment to a patient.

In one embodiment of the present invention, a method of determining the time of stroke symptom onset is provided comprising obtaining a biological sample from an individual; contacting the biological sample with a detection composition comprising at least one of an expression mediator of a LY96, a ARG1, a CA4, and/or a TLR expression mediator, or combinations thereof, wherein at least one of these expression mediators is associated with an acute phase response of ischemic stroke, for forming a detectable response; and correlating the detectable response with a time of onset of one or more stroke symptoms.

Another embodiment of this invention provides a method of determining the time of stroke symptom onset comprising obtaining a biological sample from an individual; contacting the biological sample with a panel of detectable polynucleotides or functional polynucleotide fragments which correspond to at least one or more of an expression mediator of a LY96, a ARG1, a CA4, and/or a TLR expression mediator, or combinations thereof, wherein at least one of these expression mediators is associated with an acute phase response of ischemic stroke, for forming a detectable response; and correlating the detectable response with a time of onset of one or more stroke symptoms.

In yet another embodiment of this invention, a method is provided for determining the time of stroke symptom onset comprising obtaining a biological sample from an individual; contacting the biological sample with a panel of detectable oligonucleotides which correspond to at least one or more of an expression mediator of a LY96, a ARG1, a CA4, and/or a TLR expression mediator, or combinations thereof, wherein at least one of the expression mediators is associated with an acute phase response of ischemic stroke, for forming a detectable response; and correlating the detectable response with a time of onset of one or more stroke symptoms.

Another embodiment of this invention provides a method of determining the time of stroke symptom onset comprising obtaining a biological sample from an individual; contacting the biological sample with a panel of detectable antibodies for at least one of an expression mediator that is a LY96, a ARG1, a CA4, and/or a TLR expression mediator, or combinations thereof, wherein at least one of the expression mediators is associated with an acute phase response of ischemic stroke, for forming a detectable response; and correlating the detectable response with a time of onset of one or more stroke symptoms.

In another embodiment a method is provided for determining the time of stroke symptom onset comprising creating a sample by extracting target polynucleotide molecules from an individual afflicted with an ischemic stroke so that the DNA is preserved, deriving the mRNA from the RNA of the individual, labeling the mRNA and hybridizing to a detection mechanism containing at least one of an expression mediator that is at least one of a LY96, a ARG1, a CA4, and/or a TLR expression mediator, wherein at least one of the expression mediators is associated with an acute phase response of ischemic stroke, for forming a detectable response; and correlating the detectable response with a time of onset of one or more stroke symptoms.

In addition, the invention is directed to compositions that detect the biomarkers. The present invention provides compositions, including nucleic acid probes and antibodies that are complementary or specific to biomarkers that are associated with acute phase response of ischemic stroke.

Another embodiment of the present invention provides a composition for the detection of biomarkers comprising a nucleic acid probe that is specific for at least one of a LY96, ARG1, CA4, and/or TLR expression mediator.

Another embodiment of the present invention provides a composition for the detection of biomarkers comprising at least one antibody that is specific for at least one of a LY96, ARG1, CA4, and/or TLR expression mediator.

Another embodiment of this invention provides a composition comprising a purified biomarker specific for at least one of a LY96, ARG1, CA4, and/or TLR expression mediator and the corresponding encoding nucleic acids thereof.

In yet another embodiment of this invention, a method is disclosed for determining the time of onset of ischemic stroke symptoms or other neurological disease comprising creating a sample by extracting target polynucleotide molecules from an individual afflicted with an ischemic stroke so that the RNA is preserved, deriving the nucleic acids from the mRNA of the individual, labeling the nucleic acids and hybridizing to a detection mechanism containing at least one or more of SEQ ID NO: 1, SEQ ID NO: 2, SEQ ID NO: 3, SEQ ID NO: 4, SEQ ID NO: 5, SEQ ID NO:6, SEQ ID NO:7 and SEQ ID NO:8; determining a chemoresponse based on gene expression profiles between the sample and the detection mechanism; and correlating the chemoresponse with a time of onset of one or more stroke symptoms or one or more symptoms of a neurological disease.

Another embodiment of this invention, a method is disclosed for determining the time of onset of ischemic stroke symptoms or other neurological disease comprising creating a sample by extracting target polynucleotide molecules from an individual afflicted with an ischemic stroke so that the RNA is preserved, deriving the nucleic acids from the mRNA of the individual, labeling the nucleic acids and hybridizing the labeled nucleic acids to a detection mechanism containing probes that are a portion of at least one or more of SEQ ID NO:2, SEQ ID NO:3, SEQ ID NO:5, SEQ ID NO:6 and SEQ ID NO:8; determining a chemoresponse based on gene expression profiles between the sample and said detection mechanism; and correlating said chemoresponse with a time of onset of one or more stroke symptoms or one or more symptoms of neurological disease.

The neurological disease is selected from the group consisting essentially of at least one of multiple sclerosis, Alzheimer's disease, migraine, epilepsy, and traumatic brain injury.

The SEQ ID NO:1 is the Sequence ID for the marker Lymphocyte antigen 96 (LY96) [Homo sapiens] Gene ID: 23643 The SEQ ID NO:2 is the Sequence ID for the marker Lymphocyte antigen 96, transcript variant 1. The SEQ ID NO:3 is the Sequence ID for the marker Lymphocyte antigen 96 also known as MD2, transcript variant 2. The SEQ ID NO:4 is the Sequence ID for the marker ARG1 arginase 1 [Homo sapiens (human)] Gene ID: 383. The SEQ ID NO:5 is the Sequence ID for the marker arginase 1 (ARG1), transcript variant 1, mRNA. The SEQ ID NO:6 is the Sequence ID for the marker arginase 1 (ARG1), transcript variant 2, mRNA. The SEQ ID NO:7 is the Sequence ID for the marker CA4 carbonic anhydrase IV [Homo sapiens (human)] Gene ID: 762. The SEQ ID NO:8 is the Sequence ID for the marker carbonic anhydrase IV (CA4), mRNA. These SEQ IDs are available to those persons skilled in the art and are disclosed herein.

### BRIEF DESCRIPTION OF THE DRAWINGS

FIG. 1(a) is a table that sets forth patient demographic information; FIG. 1(b) is a graph of the expression of LY96 in peripheral blood (patients-human beings) in first 48 hours after stroke which shows that an increased time from stroke onset is associated with decrease expression of LY96; FIG. 1(c) is a graph of LY96 Ct gene expression over time that shows reverse transcription polymerase chain reaction (RT-PCR) validation of LY96 wherein the LY96 raw Ct values show a decreasing trend over time with a small sample size; FIG. 1(d) is a graph of LY96 dCt gene expression over time that shows RT-PCR validation of LY96 when normalizing LY96 to B-Actin the decreasing trend is no longer seen.

FIG. 2 is a Sequence ID for the marker Lymphocyte antigen 96 (LY96) [Homo sapiens] Gene ID: 23643.

FIG. 3 is a Sequence ID for the marker Lymphocyte antigen 96, transcript variant 1.

FIG. 4 is a Sequence ID for the marker Lymphocyte antigen 96 also known as MD2, transcript variant 2.

FIG. 5 is a Sequence ID for the marker ARG1 arginase 1 [Homo sapiens (human)] Gene ID: 383.

FIG. 6 is a Sequence ID for the marker arginase 1 (ARG1), transcript variant 1, mRNA.

FIG. 7 is a Sequence ID for the marker arginase 1 (ARG1), transcript variant 2, mRNA.

FIG. 8 is a Sequence ID for the marker CA4 carbonic anhydrase IV [Homo sapiens (human)] Gene ID: 762.

FIG. 9 is a Sequence ID for the marker carbonic anhydrase IV (CA4), mRNA.

FIGS. 10(a)-(l) are graphs that show data for patient populations (human beings) of various age groups (i.e. less than 60 years old, greater than 60 years old, less than 80 years old, and greater than 80 years old, respectively) plotted as the expression (see the y axis of each graph) of a specific expression mediator of the present invention over time (in hours, from zero hours to 48 hours) (see the x-axis of each graph). FIG. 10(a) shows expression of LY96 for patients less than 60 years of age. FIG. 10(b) shows expression of LY96 for patients greater than 60 years of age. FIG. 10(c) shows expression of ARG1 for patients less than 60 years of age. FIG. 10(d) shows expression of ARG1 for patients greater than 60 years of age. FIG. 10(e) shows expression of CA4 for patients less than 60 years of age. FIG. 10(f) shows expression of CA4 for patients greater than 60 years of age. FIG. 10(g) shows expression of ARG1 for patients less than 80 years of age. FIG. 10(h) shows expression of ARG1 for patients greater than 80 years of age. FIG. 10(i) shows expression of CA4 for patients less than 80 years of age. FIG. 10(j) shows expression of CA4 for patients greater than 80 years of age. FIG. 10(k) shows expression of LY96 for patients less than 80 years of age. FIG. 10(l) shows expression of LY96 for patients greater than 80 years of age. CA4 and ARG1 expression significantly decreased >1.5 fold between baseline and follow up. These decreases in expression were associated with an increase from time of stroke onset and were significantly lower in older aged patients (patients greater than 80 years of age).

### DETAILED DESCRIPTION OF THE INVENTION

### Definitions

The present invention may be understood more readily by reference to the following detailed description of preferred embodiments of the invention and the Methods included therein. Before the present methods and techniques are disclosed and described, it is to be understood that this invention is not limited to specific analytical or synthetic methods as such may, of course, vary. It is also to be understood that the terminology used herein is for the purpose of describing particular embodiments only and is not intended to be limiting. Unless defined otherwise, all technical and scientific terms used herein have the meaning commonly understood by one of ordinary skill in the art to which this invention belongs.

As used herein and in the claims, the singular forms “a,” “and,” and “the” include plural reference unless the context clearly dictates otherwise. Thus, for example, reference to “a biomarker” is reference to one or more biomarkers and includes equivalents thereof known to those skilled in the art.

The term “antibody,” as used herein, refers to immunoglobulin molecules and immunologically active portions of immunoglobulin molecules. As such, the term antibody can refer to any type, including for example IgG, IgE, IgM, IgD, IgA and IgY, any class, including for example IgG1, IgG2, IgG3, IgG4, IgA1 and IgA2 or subclass of immunoglobulin molecules. Further, the terms “antibody” and immunoglobulin” can be used interchangeably throughout the specification. Antibodies or immunoglobulins can be used to encompass not only whole antibody molecules, but also antibody multimer, antibody fragments as well as variants of antibodies, antibody multimers and antibody fragments. The immunoglobulin molecules can be isolated from nature or prepared by recombinant means or chemically synthesized. Antibodies and immunoglobulins of the invention can be used for various purposes. In a preferred embodiment, antibodies and immunoglobulins can be used for the detection of the biomarkers through the use of any suitable detection mechanism, e.g. ELISA.

The terms “ischemic stroke (IS)”, “acute ischemic stroke (AIS)”, and “Acute Ischemic Cerebrovascular Syndrome (AICS)” are used interchangeably and refer to the condition of a patient experiencing a rapid loss of brain function due to disturbance in the blood supply to the brain. The diagnostic criteria of AICS defined by Kidwell et. al. “Acute Ischemic Cerebrovascular Syndrome: Diagnostic Criteria,” Stroke, 2003, 34, pp. 2995-2998 (incorporated herein by reference) are as follow:
Definite AICS: Acute onset of neurologic dysfunction of any severity consistent with focal brain ischemia AND imaging/laboratory CONFIRMATION of an acute vascular ischemic pathology.
Probable AICS: Acute onset of neurologic dysfunction of any severity suggestive of focal brain ischemic syndrome but WITHOUT imaging/laboratory CONFIRMATION of acute ischemic pathology (diagnostic studies were negative but INSENSITIVE for ischemic pathology of the given duration, severity and location). Imaging, laboratory, and clinical data studies do not suggest nonischemic etiology: possible alternative etiologies ARE ruled out.
Possible AICS: Acute neurologic dysfunction of any duration or severity possibly consistent with focal brain ischemia WITHOUT imaging/laboratory CONFIRMATION of acute ischemic pathology (diagnostic studies were not performed or were negative and SENSITIVE for ischemic pathology of the given duration, severity and location). Possible alternative etiologies are NOT ruled out. Symptoms may be nonfocal or difficult to localize.
Not AICS: Acute onset of neurologic dysfunction with imaging/laboratory CONFIRMATION of NONISCHEMIC pathology (including normal imaging/laboratory studies that are highly sensitive for ischemic pathology of the given duration, severity, and location) as the cause of the neurologic syndrome.

The term “stroke symptoms” can refer to those symptoms that may present at the onset of any type of stroke, including acute ischemic stroke. Stroke symptoms include those recognized by the National Stroke Association (www.stroke.org), which are as follows: (a) Sudden numbness or weakness of face, arm or leg-especially on one side of the body, (b) Sudden confusion, trouble speaking or understanding, (c) Sudden trouble seeing in one or both eyes, (d) Sudden trouble walking, dizziness, loss of balance or coordination, and (e) Sudden severe headache with no known cause.

The term “diagnosis” refers to methods by which one skilled in the art can estimate and/or determine whether or not a patient is suffering for, or is at some level of risk of developing, a given disease or condition. The skilled artisan, e.g. stroke clinician or point of care physician, often makes a diagnosis on the basis of one or more diagnostic indicators, i.e., a biomarker, the risk, presence, absence, or amount of which is indicative of the presence, severity, or absence of the condition, e.g., acute ischemic stroke or other neurological condition.

The phrase “acute phase response” as used herein refers to a group of physiological processes occurring soon after the onset of infection, trauma, e.g. ischemic stroke, inflammatory processes, and some malignant conditions. Acute phase response includes the increase of acute phase proteins in serum, fever, increased vascular permeability, and metabolic and pathologic changes. Biomarkers associated with acute phase response include, but are not limited to, LY96, ARG1, CA4, and TLR.

The terms “biomarker”, “marker”, and “expression mediator” are used interchangeable herein and refers to molecules (e.g. proteins, polypeptides, polynucleotides, oligonucleotides, mRNA, genomic DNA or DNA transcripts) found in the body (e.g. blood, other body fluids, or tissues) that is correlated with a normal or abnormal condition. In a preferred embodiment of the invention, the terms biomarker, marker and expression mediator refers to proteins, polypeptides, polynucleotides, oligonucleotides, mRNA, genomic DNA and DNA transcripts that are associated with acute phase response due to acute ischemic stroke or other neurological diseases or conditions. Further, biomarker, marker, and expression mediator may refer to RNA expression, metabolites, protein expression, or other upstream or downstream mediators. In another embodiment of the invention, the terms biomarker, marker and expression mediator refers to the complementary sequences of mRNA or DNA of a biomarker. Specific biomarkers of acute phase response due to acute ischemic stroke identified by the invention include lymphocyte antigen 96 (LY96), arginase 1 (ARG1), carbonic anhydrase 4 (CA4), and toll-like receptors (TLR) and upstream or downstream mediators of LY96, ARG1, CA4 and TLR. These specific biomarkers are described in detail hereinafter. As such, expression mediators can include RNA expression, metabolites, protein expression, or other upstream or downstream mediators associated with LY96, ARG1, CA4 and/or TLR. For example, a biomarker of the invention can include mRNA encoding LY96, ARG1, CA4, and/or TLR. In another example, an expression mediator of the invention can include nucleotides complementary or homologous to a portion of the mRNA of LY96, ARG1, CA4, and/or TLR. In yet another example, an expression mediator of the invention can include nucleotides complementary or homologous to a portion of the genomic DNA of LY96, ARG1, CA4 and/or TLR. The length of complementary or homologous nucleotides can be any length. In one embodiment of the present invention, the length of complementary or homologous nucleotides to mRNA or genomic DNA of LY96, ARG1, CA4 and/or TLR is from about 10 to about 15 nucleotides. In another embodiment, the length of complementary or homologous nucleotides to mRNA or genomic DNA of LY96, ARG1, CA4 and/or TLR is from about 15 to about 20 nucleotides. In yet another embodiment, the length of complementary or homologous nucleotides to mRNA or genomic DNA of LY96, ARG1, CA4 and/or TLR is from about 20 to about 25 nucleotides. In another embodiment, the length of complementary or homologous nucleotides to mRNA or genomic DNA of LY96, ARG1, CA4 and/or TLR is from about 20 to about 30 nucleotides. In yet another embodiment, the length of complementary or homologous nucleotides to mRNA or genomic DNA of LY96, ARG1, CA4 and/or TLR is from about 30 to about 40 nucleotides. In another embodiment, the length of complementary or homologous nucleotides to mRNA or genomic DNA of LY96, ARG1, CA4 and/or TLR is from about 40 to about 50 nucleotides. In yet another embodiment, the length of complementary or homologous nucleotides to mRNA or genomic DNA of LY96, ARG1, CA4 and/or TLR is from about 50 to about 75 nucleotides. In another embodiment, the length of complementary or homologous nucleotides to mRNA or genomic DNA of LY96, ARG1, CA4 and/or TLR is from about 75 to about 100 nucleotides. In yet another embodiment, the length of complementary or homologous nucleotides to mRNA or genomic DNA of LY96, ARG1, CA4 and/or TLR s is from about 100 to about 150 nucleotides. In another embodiment, the length of complementary or homologous nucleotides to mRNA or genomic DNA of LY96, ARG1, CA4 and/or TLR is from about 150 to about 200 nucleotides. In yet another embodiment, the length of complementary or homologous nucleotides to mRNA or genomic DNA of LY96, ARG1, CA4 and/or TLR is from about 200 to about 250 nucleotides. In another embodiment, the length of complementary or homologous nucleotides to mRNA or genomic DNA of LY96, ARG1, CA4 and/or TLR is from about 250 to about 300 nucleotides. In yet another embodiment, the length of complementary or homologous nucleotides to mRNA or genomic DNA of LY96, ARG1, CA4 and/or TLR is more than 300 nucleotides. Additional biomarkers may also be included in the invention. Biomarkers can be detected, identified, or measure using any suitable methods, mechanisms or instrumentation for detecting, identifying or detecting polypeptides, proteins, or nucleic acid molecules including mRNA, genomic DNA and transcribed DNA. Specific detection mechanisms that can detect, identify or measure biomarkers are described in detail hereinafter.

The term “proteins” and “polypeptides” used as biomarkers herein are intended to include any fragments thereof, in some particular embodiment, immunologically detectable fragments. A skilled artisan would recognize that proteins which are released by cells may become damaged during an acute phase response (e.g., as a result of an acute ischemic stroke) could become degraded or cleaved into such fragments. Further, some markers are synthesized in an inactive form, which may be subsequently activated, e.g., by proteolysis.

The phrases “detection mechanism” and “detection assay” are used interchangeably and used herein are intended any standard comparison mechanism or tool comprising biomarkers described above. Also, the term “detection mechanism” is used herein to refer to any standard comparison mechanism or tool to measure, identify or detect biomarkers. As such, the term detection mechanism may refer to a microarray or an assay of reverse transcription polymerase chain reaction (RT-PCR). Further, the term detection mechanism may refer to panel of antibodies that recognize specific biomarkers. In one embodiment of the invention, detection mechanism refers to a microarray comprising at least one of the biomarkers described herein. In a preferred embodiment of the invention, the detection mechanism refers to a microarray, RT-PCR assay, or probe set comprising at least one of the biomarkers of LY96, ARG1, CA4, and/or TLR. Further, detection mechanism can refer to analyzing biomarkers that are nucleic acid molecules. For example, detecting or measuring mRNA molecules in peripheral blood encoding a biomarker of the invention is a type of detection mechanism. Additionally, “gene panel” is similarly used herein to refer to a detection mechanism to measure, identify or detect biomarkers.

Additionally, the term “filament-based diagnostic system” used herein refers to a specific detection mechanism that is known in the art. Filament-based diagnostic system includes, but is not limited to, a material (e.g., polyester filament or gold wire) that is used to capture or bind to biomarkers collected from a biological sample. Generally, filament-based diagnostic system may either capture antibodies on a polyester filament, or DNA (or other nucleic acid) probe on a gold wire, each of which function as molecular hooks to troll for polypeptides or nucleic acid molecules of interest (e.g. the biomarker polypeptides of the current invention, or their corresponding mRNA molecules) in a biological sample, for example but not limited to peripheral blood of a patient (“patient” means any animal or creature warm or cold blooded, including such as for example but not limited to a human being). For antibody detection of target polypeptides (e.g. the biomarker polypeptides of the current invention), a filament material immobilized with antibodies specific for the target polypeptides that have been exposed to a test biologic sample is threaded through an array of chambers that carry out the washing and then a reporting of the results therefrom. For nucleic acid detection (e.g. mRNA encoding the biomarkers of the current invention), a filament containing DNA or nucleotide probes bound to the filament (for example, a gold filament) that are specific or hybridize to target nucleic acid molecules in the biologic sample (e.g. mRNA of each biomarker in the biologic sample) that is passed through various chambers that carry out the washing and then the reporting of any probe/target interactions that have occurred on the filament surface. Those persons skilled in the art understand what is meant by a “filament-based diagnostic system” and recognize that the filament may be made of various materials, such as for example, but not limited to, polystyrene, glass, and nylon. U.S. patent application Ser. No. 13/580,571 (US Patent Application Publication No. US 2013/0189243 A1, published Jul. 25, 2013) sets forth a general description of a filament-based diagnostic system, and such description is incorporated by reference herein.

By the terms “detect,” “detection,” “detectable,” “detectable response” and “detecting” are intended to refer to the identification of the presence, absence, or quantity of a given biomarker. As such, the terms “detectable composition,” “detectable polynucleotides,” “detectable oligonucleotides,” and “detectable antibodies” are intended to refer to the identification of the presence, absence, or quantity of a biomarker that is represented by a composition, polynucleotides, oligonucleotides and antibodies, respectively.

As used herein, the term “correlate” means to bring at least two factors into complementary, parallel, or reciprocal relation. For example, the detectable response is correlated to the time of onset of acute ischemic stroke symptoms. In a specific embodiment, the expression level of biomarkers of acute phase response, e.g. LY96, ARG1, CA4 and/or TLR, are correlated to the time of onset of stroke symptoms or other neurological disease symptoms. The instant invention establishes the correlation between biomarkers and time of onset of stroke or neurological disease symptoms (see Methods). Further, the present invention correlates sets of data (i.e. biomarker expression and time of onset of stroke or neurological disease symptoms) by means of an algorithm. These algorithms are well known in the art and are discussed further herein (see Methods).

As used herein, the terms “biological sample,” “patient sample” or “sample” refer to a sample obtained from an organism or from components (e.g., cells) of a subject or patient for the purpose of diagnosis, prognosis, or evaluation of subject of interest. As used herein to term “patient” or “individual” means any animal or creature, warm or cold blooded, including for example but not limited to, a human being. In certain embodiments, such a sample may be obtained for the purpose of determining the outcome of an ongoing condition or the effect of a treatment regimen on a condition. The sample may be of any biological tissue or fluid. The sample may be a clinical sample which is a sample derived from a patient. Such samples include, but are not limited to, brain cells or tissues, cerebrospinal fluid, nerve tissue, sputum, blood, serum, plasma, blood cells (e.g., white cells), tissue samples, biopsy samples, urine, peritoneal fluid, and pleural fluid, saliva, semen, breast exudate, tears, mucous, lymph, cytosols, ascites, amniotic fluid, bladder washes, and bronchioalveolar lavages or cells therefrom, among other body fluid samples. Preferably, the sample is peripheral blood. Preferable, the sample contains one or more of the biomarkers of the invention. The patient sample may be fresh or frozen, and may be treated, e.g. with heparin, citrate or EDTA. Samples may also include sections of tissues such as frozen sections taken for histological purposes.

### Biomarkers:

The present invention identifies gene profiles and correlates each with determining the onset of time of an acute phase of ischemic stroke or other neurological event. At least one of these genes physiological corresponds to the acute phase response. Specifically, the present invention determines the expression of at least one of the markers (i.e. Lymphocyte antigen 96 (LY96) aka MD2; carbonic anhydrase 4 (CA4), Arginase 1 (ARG1), or toll-like receptors (TLR), or a combination of at least two of the expression mediators selected from the group of Lymphocyte antigen 96 (LY96) aka MD2; carbonic anhydrase 4 (CA4), Arginase 1 (ARG1), or toll-like receptors (TLR)) that is/are associated with the time from when the ischemic event began, and thus a surrogate for when the stroke symptoms or other symptoms of a neurological disease began. The present invention discloses the functional relationship of a one or more gene panels that includes, for example, at least one of LY96, ARGI, and CA4 (i.e. markers) with time of stroke symptom onset.

In one embodiment of the present invention, a method of determining the time of stroke symptom onset is provided comprising obtaining a biological sample from an individual; contacting the biological sample with a detection composition comprising at least one of an expression mediator that is at least one of LY96, ARGI, CA4, and/or TLR expression mediators, or a combination of these expression mediators, wherein at least one of these expression mediators is associated with an acute phase response of ischemic stroke, for forming a detectable response; and correlating the detectable response with a time of onset of one or more stroke symptoms.

As used herein, the term “combination” means two or more specific expression mediators, such as for example but not limited to, the combination of LY96 and ARGI, or the combination of LY96 and CA4, or the combination of LY96, ARGI, and CA4, or the combination of CA4 and ARGI, or a combination of a TLR expression mediator and CA4, or a combination of ARGI and a TLR expression mediator, to name a few of such exemplary combinations.

In a preferable embodiment of this invention, this method, as described herein, of determining the time of stroke symptom onset is provided comprising obtaining a biological sample from an individual; contacting the biological sample with a detection composition comprising at least one of an expression mediator that is selected from the group consisting of a LY96, an ARGI, a CA4, and a TLR expression mediator, or a combination of at least two of a LY96, an ARGI, a CA4, and a TLR expression mediator, wherein at least one of these expression mediators is associated with an acute phase response of ischemic stroke, for forming a detectable response; and correlating the detectable response with a time of onset of one or more stroke symptoms. In a more preferable embodiment of this invention,this method, as described herein, of determining the time of stroke symptom onset is provided comprising obtaining a biological sample from an individual; contacting the biological sample with a detection composition comprising at least one of an expression mediator that is selected from the group consisting of a LY96, an ARGI, and a CA4 expression mediator, or a combination of at least two of LY96, ARGI, and CA4, wherein at least one of these expression mediators is associated with an acute phase response of ischemic stroke, for forming a detectable response; and correlating the detectable response with a time of onset of one or more stroke symptoms. In a most preferable embodiment of this invention, this method, as described herein, of determining the time of stroke symptom onset is provided comprising obtaining a biological sample from an individual; contacting the biological sample with a detection composition comprising at least one of an expression mediator that is selected from the group consisting of a LY96, an ARGI, and a CA4 expression mediator, or a combination of each of LY96, ARGI, and CA4 expression mediators, wherein at least one of these expression mediators is associated with an acute phase response of ischemic stroke, for forming a detectable response; and correlating the detectable response with a time of onset of one or more stroke symptoms.

Another embodiment of this invention provides a method of determining the time of stroke symptom onset comprising obtaining a biological sample from an individual; contacting the biological sample with a panel of detectable polynucleotides or functional polynucleotide fragments which correspond to at least one (or more) of a LY96, ARGI, CA4, and/or TLR expression mediators, wherein at least one of the expression mediators is associated with an acute phase response of ischemic stroke, for forming a detectable response; and correlating the detectable response with a time of onset of one or more stroke symptoms.

In yet another embodiment of this invention, a method is provided for determining the time of stroke symptom onset comprising obtaining a biological sample from an individual; contacting the biological sample with a panel of detectable oligonucleotides which correspond to at least one or more of a LY96, ARGI, CA4, and/or TLR expression mediators, wherein at least one of the expression mediators is associated with an acute phase response of ischemic stroke, for forming a detectable response; and correlating the detectable response with a time of onset of one or more stroke symptoms.

Another embodiment of this invention provides a method of determining the time of stroke symptom onset comprising obtaining a biological sample from an individual; contacting the biological sample with a panel of detectable antibodies for one or more of a LY96, ARG1, CA4, and/or TLR expression mediators, wherein at least one of the expression mediators is associated with an acute phase response of ischemic stroke, for forming a detectable response; and correlating the detectable response with a time of onset of one or more stroke symptoms.

In another embodiment a method is provided for determining the time of stroke symptom onset comprising creating a sample by extracting target polynucleotide molecules from an individual afflicted with an ischemic stroke so that the RNA is preserved, deriving the mRNA from the RNA of the individual, labeling the mRNA and hybridizing to a detection mechanism containing at least one of the LY96, ARG1, CA4, and/or TLR expression mediators, wherein at least one of the expression mediators is associated with an acute phase response of ischemic stroke, for forming a detectable response; and correlating the detectable response with a time of onset of one or more stroke symptoms.

Another embodiment of the present invention provides a composition for the detection of biomarkers comprising a nucleic acid probe that is specific for at least one of a LY96, ARG1, CA4, and/or TLR expression mediator.

Another embodiment of the present invention provides a composition for the detection of biomarkers comprising at least one antibody that is specific for at least one of a LY96, ARG1, CA4, and/or TLR expression mediator.

Another embodiment of this invention provides a composition comprising a purified biomarker specific for at least one of a LY96, ARG1, CA4, and/or TLR expression mediator and the corresponding encoding nucleic acids thereof.

In a preferred embodiment of this invention, a method of determining the time of stroke symptom onset is provided comprising obtaining a biological sample from an individual; contacting the biological sample with a panel of detectable polynucleotides or functional polynucleotide fragments which correspond to at least one expression mediator selected from the group consisting of a LY96, an ARG1, and a CA4, or a combination of these expression mediators, wherein at least one of these expression mediators is associated with an acute phase response of ischemic stroke; forming a detectable response; and correlating the detectable response with a time of onset of one or more stroke symptoms.

In a preferred embodiment of this invention, a method of determining the time of stroke symptom onset is provided comprising obtaining a biological sample from an individual; contacting the biological sample with a panel of detectable oligonucleotides which correspond to at least one expression mediator selected from the group consisting of a LY96, an ARG1, and a CA4, or a combination of these expression mediators, wherein at least one of these expression mediators is associated with an acute phase response of ischemic stroke; forming a detectable response; and correlating the detectable response with a time of onset of one or more stroke symptoms.

In a preferred embodiment of this invention, a method of determining the time of stroke symptom onset is provided comprising obtaining a biological sample from an individual; contacting the biological sample with a panel of detectable antibodies for at least one expression mediator selected from the group consisting of a LY96, an ARG1, and a CA4, or a combination of these expression mediators, wherein at least one of these expression mediators is associated with an acute phase response of ischemic stroke; forming a detectable response; and correlating the detectable response with a time of onset of one or more stroke symptoms.

In a preferred embodiment of this invention, a method of determining the time of stroke symptom onset is provided comprising treating a sample by extracting target polynucleotide molecules from an individual afflicted with an ischemic stroke so that the RNA is preserved, deriving the mRNA from the mRNA of the individual, labeling the mRNA and hybridizing to a detection mechanism containing at least one expression mediator selected from the group consisting of a LY96, an ARG1, and a CA4, or a combination of these expression mediators, wherein at least one of these expression mediators is associated with an acute phase response of ischemic stroke; forming a detectable response; and correlating the detectable response with a time of onset of one or more stroke symptoms.

In a preferred embodiment of this invention, a composition for the detection of biomarkers is provided comprising a nucleic acid probe that is specific for at least one expression mediator selected from the group consisting of a LY96, an ARG1, and a CA4, or combinations of these expression mediators.

In another preferred embodiment of this invention, a composition for the detection of biomarkers is provided comprising at least one antibody that is specific for at least one expression mediator that is selected from the group consisting of a LY96, an ARG1, and a CA4, or a combination of these expression mediators.

In yet another preferred embodiment of this invention, a composition is provided comprising a purified biomarker specific for at least one expression mediator selected from the group consisting of a LY96, an ARG1, and a CA4 expression mediators, or a combination of these expression mediators, and the corresponding encoding nucleic acids thereof.

In yet another embodiment of this invention, a method is disclosed for determining the time of onset of ischemic stroke symptoms or other neurological disease comprising creating a sample by extracting target polynucleotide molecules from an individual afflicted with an ischemic stroke so that the RNA is preserved, deriving the nucleic acids from the mRNA of the individual, labeling the nucleic acids and hybridizing to a detection mechanism containing at least one or more of SEQ ID NO:1, SEQ ID NO:2, SEQ ID NO:3, SEQ ID NO:4, SEQ ID NO:5, SEQ ID NO:6, SEQ ID NO:7, and SEQ ID NO:8; determining a chemoresponse based on gene expression profiles between the sample and the detection mechanism; and correlating the chemoresponse with a time of onset of one or more stroke symptoms or one or more symptoms of a neurological disease.

In another embodiment of this invention, a method is disclosed for determining the time of onset of ischemic stroke symptoms or other neurological disease comprising creating a sample by extracting target polynucleotide molecules from an individual afflicted with an ischemic stroke so that the RNA is preserved, deriving the nucleic acids from the mRNA of the individual, labeling the nucleic acids and hybridizing to a detection mechanism containing at least one or more of, SEQ ID NO:2, SEQ ID NO:3, SEQ ID NO:5, SEQ ID NO:6, and SEQ ID NO:8; determining a chemoresponse based on gene expression profiles between the sample and the detection mechanism; and correlating the chemoresponse with a time of onset of one or more stroke symptoms or one or more symptoms of a neurological disease.

The neurological disease is selected from the group consisting essentially of at least one of multiple sclerosis, Alzheimer's disease, migraine, epilepsy, and traumatic brain injury.

The SEQ ID NO:1 is the Sequence ID for the marker Lymphocyte antigen 96 (LY96) [Homo sapiens] Gene ID: 23643 The SEQ ID NO:2 is the Sequence ID for the marker Lymphocyte antigen 96, transcript variant 1. The SEQ ID NO:3 is the Sequence ID for the marker Lymphocyte antigen 96 also known as MD2, transcript variant 2. The SEQ ID NO:4 is the Sequence ID for the marker ARG1 arginase 1 [Homo sapiens (human)] Gene ID: 383. The SEQ ID NO:5 is the Sequence ID for the marker arginase 1 (ARG1), transcript variant 1, mRNA. The SEQ ID NO:6 is the Sequence ID for the marker arginase 1 (ARG1), transcript variant 2, mRNA. The SEQ ID NO:7 is the Sequence ID for the marker CA4 carbonic anhydrase IV [Homo sapiens (human)] Gene ID: 762. The SEQ ID NO:8 is the Sequence ID for the marker carbonic anhydrase IV (CA4), mRNA.

The compositions and methods of the present invention may be used as follows:
1. As a marker or predictor of time of human ischemic stroke onset.
2. As a marker or predictor of time of symptom onset in other neurological diseases (multiple sclerosis; Alzheimer's disease; migraine; epilepsy; traumatic brain injury, etc.).
3. As a novel therapeutic target for stroke treatment.
4. As a novel therapeutic target for treatment of other neurological diseases (multiple sclerosis; Alzheimer's disease; migraine; epilepsy; traumatic brain injury; etc.).
5. As a marker of brain tissue injury or predictor of time.
6. As a prognostic indicator of health outcome following neurologic injury.
7. As a method to increase the time window for tPA or other lytic drug treatment.

The present invention solves an existing problem in determining the difficult clinical assessment of time of stroke symptom onset. This assessment is problematic to determine either because the patient is incoherent or the event is not witnessed. An unbiased surrogate of time of symptom onset would improve clinical evaluation and may even facilitate increased utilization of tPA or other lytic agents/procedures.

For the purpose of determining time of symptom onset, after clinical validation, the present invention provides a method as a point of care test. Therefore the expression of LY96, ARG1 and/or CA4 either through RNA expression, metabolites, protein expression, or other upstream or downstream mediators associated with LY96, ARG1 and/or CA4 expression would be analyzed real-time for clinical decision making. It may also be used in combination with other markers of the acute phase response, such as for example toll-like receptors (TLR) or damage or pathogen associated molecular patterns (DAMPs and PAMPs). Those persons skilled in the art understand that LY96 is an example of a TLR expression mediator. Other examples of TLR expression mediators are known by those skilled in the art including those associated with TLR1 and TLR2.

Since LY96, ARG1 and CA4 are markers of the acute phase response and a general response to stress, it is possible the level of expression can be used to determine disease severity or time of symptom onset in multiple instances (acute or chronic neurological diseases, cardiac disease or trauma/traumatic events).

In one aspect, the present invention provides a biomarker for use in methods for diagnosing stroke and/or determining the time of stroke symptom onset. In addition, the present invention is directed to compositions (e.g., arrays, probes, biomarker panels) that comprise LY96, ARG1 and/or CA4 or TLR expression or other upstream or downstream mediators associated with the acute phase response which can be used in diagnosing/prognosing stroke or time of stroke symptom onset, or continued/secondary brain damage. Further, since biomarker(s) of the present invention represent(s) a target of intervention for the treatment of stroke, the biomarker(s) of this invention can be used in methods for screening compounds or agents that can treat stroke or a symptom thereof and which are detectable by the evaluation of the biomarkers of the invention. In addition, the invention is directed to compositions that are useful in the detection of the biomarkers, including nucleic acid probes and antibodies that are specific for the biomarkers of the invention, as well as to compositions comprising purified biomarkers and their corresponding encoding nucleic acid molecules.

In one aspect, the invention provides a method for determining time of stroke symptom onset or stroke in a subject presenting symptoms characteristic of a stroke or at risk of having a stroke or other neurological disease, comprising:

(a) obtaining a biological sample from the patient;

(b) contacting the biological sample with detection means capable of detecting the presence of LY96 or TLRs. The detection means is a detection mechanism as described herein.

In other aspects, the invention provides a kit comprising a means for detecting at least one of LY96, ARG1, CA4, or a TLR, or a combination thereof. Thus, those skilled in the art will understand that the present invention provides a kit comprising a detecting mechanism for detecting at least one biomarker that is diagnostic of an ischemic stroke, said biomarker selected from the group consisting of a lymphocyte antigen 96 (LY96), an arginase 1 (ARG1), and a carbonic anhydrase 4 (CA4), or a combination of said biomarkers. The detecting mechanism is described herein.

In certain other aspects, the invention provides a diagnostic system comprising a panel of detectable polypeptides or functional polypeptide fragments thereof each corresponding to LY96, ARG1 and/or CA4 or TLRs.

In still other aspects, the invention provides a filament-based diagnostic system comprising a panel of detectable oligonucleotides for LY96, ARG1 and/or CA4 or TLRs.

In still further aspects, the invention provides a filament-based diagnostic system comprising a panel of detectable antibodies for LY96, ARG1 and/or CA4 or TLRs.

Those persons skilled in the art will understand that the present invention provides a filament-based diagnostic system comprising either (i) a panel of detectable polypeptides or functional polypeptide fragments thereof each corresponding to, (ii) a panel of detectable oligonucleotides each corresponding to, or (iii) a panel of detectable antibodies, each capable of specifically binding, an ischemic stroke biomarker selected from the group consisting of a lymphocyte antigen 96 (LY96), an arginase 1 (ARG1), and a carbonic anhydrase 4 (CA4), or a combination of said biomarkers.

Specifically, four biomarkers are identified in this invention: (1) Lymphocyte antigen 96 (LY96); (2) Arginase 1 (ARG1); (3) Carbonic anhydrase 4 (CA4); and (4) TLR. Each of these biomarkers is described further.

(1) Lymphocyte antigen 96 (LY96). Lymphocyte antigen 96 (LY96) is also known as MD2 protein and associates with toll-like receptor 4 (TLR4) on the cell surface. LY96 is critical for TLR4 activation as an innate response to lipopolysaccharide (LPS). Thus, LY96 provides a link between the receptor and LPS signaling. Further, TLR4 activation induces transduction pathways resulting in NF-kappaB expression and subsequent release of pro-inflammatory cytokines (e.g. IL6 and IL8). Interestingly, there evidence in the art that ischemic tissue damage is recognized on the cellular level via receptor-mediated detection of proteins (called alarmins) that are released by dead cells. Therefore, there are exogenous and endogenous systems, such as LPS and alarmins, respectively, that elicit similar responses of the innate immune system known as damage associated molecular patterns (DAMPs). The upregulation of LY96 as shown by the methods of this invention (See Methods) suggests that the response to acute ischemic stroke is mediated by the innate immune system and TLR signaling. The methods of this invention (see Methods) further shows that this up-regulation of expression of LY96 significantly decreases overtime from the onset of symptoms of an acute ischemic stroke. The human LY96 genomic sequence is publicly available as GenBank Accession No. NC_{—}000008, the complete sequences is presented herein as SEQ ID NO: 1. The human LY96 gene is disclosed as Gene ID: 23643. Further, LY96 has alternative splicing that results in multiple transcript variants encoding different isoforms. The human LY96 mRNA sequence of transcript 1 is presented herein as SEQ ID NO:2 and is publically disclosed as GenBank Accession No. NM_{—}015364. The sequence of human LY96 mRNA of transcript 2 is publically available as GenBank Accession No. NM_{—}001195797 and is disclosed herein as SEQ ID NO:3.

(2) Arginase 1 (ARG1). Arginase-1 (ARG1) is an enzyme that catalyzes the hydrolysis of L-arginine to ornithine and urea and is a critical regulator of nitric oxide (NO) synthesis. ARG1 is induced by T-helper 2 cytokines. Inflammatory stimuli result in an increased expression of inducible NO sythetase (iNOS) through L-arginine metabolism. It is possible to determine the type of inflammatory response to injury depending on the relative amount of ARG1 and iNOS, as both compete for L-arginine. Trauma is associated with an increase activity of ARG1 and a decrease in the level of arginine. In addition studies in the art suggest activation of the JAK and STAT pathways induce ARG1 in smooth muscle. Since humoral anti-inflammatory cytokines induce ARG1, the up-regulation of ARG1 (see Methods) suggests that the response to acute ischemic stroke favors an innate humoral immune response. The methods of this invention (see Methods), shows that this up-regulation of expression of ARG1 significantly decreases overtime from the onset of symptoms of an acute ischemic stroke. The human ARG1 gene is disclosed as Gene ID 383 and is publicly available as GenBank Accession No. NG_{—}007086. The full genomic sequence of ARG1 is presented herein as SEQ ID NO:4 Two transcript variants encoding different isoforms have been found for the ARG1 gene. The human ARG1 mRNA of transcript variant 1 is publicly available as GenBank Accession No. NM_{—}001244438 and is disclosed herein as SEQ ID NO:5. The human ARG1 mRNA of transcript variant 2 is publicly available as GenBank Accession No. NM_{—}000045 and is presented herein as SEQ ID NO:6.

(3) Carbonic anhydrase 4 (CA4). Carbonic anhydrase 4 (CA4) is part of a large family of zinc metalloenzymes that catalyze the reversible hydration of carbon dioxide. Hence, CA4 is crucial for all physiological processes involved in cellular respiration and transport. CA4 is a glycosylphosphatidyl-inositol-anchored membrane protein expressed on the luminal surfaces, such as pulmonary capillaries and proximal renal tubules. Thus, CA4 is found throughout the body and in the brain within the luminal surface of capillary endothelial cells. This suggests a role for CA4 in the blood brain barrier as a regulator of CO₂ and bicarbonate homeostasis in the brain. The upregulation of CA4 after an ischemic stroke, suggests there is an increase in cellular respiration that requires an increase in CA4 to convert CO₂ to HCO₃ to maintain pH. The methods of this invention (see Methods), shows that this upregulation of expression of CA4 significantly decreases overtime from the onset of symptoms of an acute ischemic stroke. The human CA4 is identified as Gene ID 762 and is publicly available as GenBank Accession No. NG_{—}012050. This genomic sequence of CA4 is presented herein as SEQ ID NO:7. The human CA4 mRNA sequence is publicly disclosed as GenBank Accession No. NM_{—}00717, the complete sequence of which is presented herein as SEQ ID NO:8.

(4) Toll-like receptors (TLR). Toll-like receptors (TLR) are a family of proteins which play a fundamental role in pathogen recognition and activation of innate immunity. TLRs mediate the production of cytokines necessary for the development of effective immunity. TLRs are single membrane-spanning, non-catalytic receptors. Activators of the TLR pathway include products of protein degradation, damaged DNA, fibrinogen and heat shock proteins through a mechanism referred to as damage associated molecular pattern (DAMPs) recognition. Bianchi ME. Damps, pams and alarmins: All we need to know about danger. J Leukoc Biol. 2007;81:1-5. Those persons skilled in the art understand that LY96 is an example of a TLR expression mediator. Other examples of TLR expression mediators are known by those skilled in the art including those associated with TLR1 and TLR2.

As stated hereinabove, Tissue plasminogen activator (tPA) has been the only FDA approved treatment for ischemic stroke since 1995. Only 2-3% of all ischemic stroke patients receive tPA because of many contraindicating factors, the first primarily being when the patient arrives at the treatment facility compared to when their symptoms began. tPA must be given within a maximum of 4.5 hours from onset of stroke symptoms. However, the median time patients arrive to the ED for treatment is around 8 hours. Increasing the time window for tPA treatment is a clinical need. In addition, up to 30% of patients are unaware of the time when their stroke symptoms began. In some cases, patients have gone to bed normal and then wake up in the morning with their symptoms. These patients cannot be given tPA because of the uncertainty surrounding the time when they were last known to be normal. The present invention recognizes the strong innate inflammatory reaction to stroke and monitors the expression of these immune genes in the peripheral blood of a patient following stroke. The present invention has found that the expression of these immune genes significantly decreases over time and thus can be used as a surrogate for when the stroke began. An unbiased measure of when stroke symptoms began would aid clinicians in their decision to treat with tPA. This could result in a 30% increase in utilization of tPA with an expected increase in functional recovery. These inflammatory immune markers may also be used to guide tPA treatment beyond the 4.5 hour time window. The methods of the present invention comprising employing these genomic biomarkers are able to guide stroke therapeutics.

### Methods:

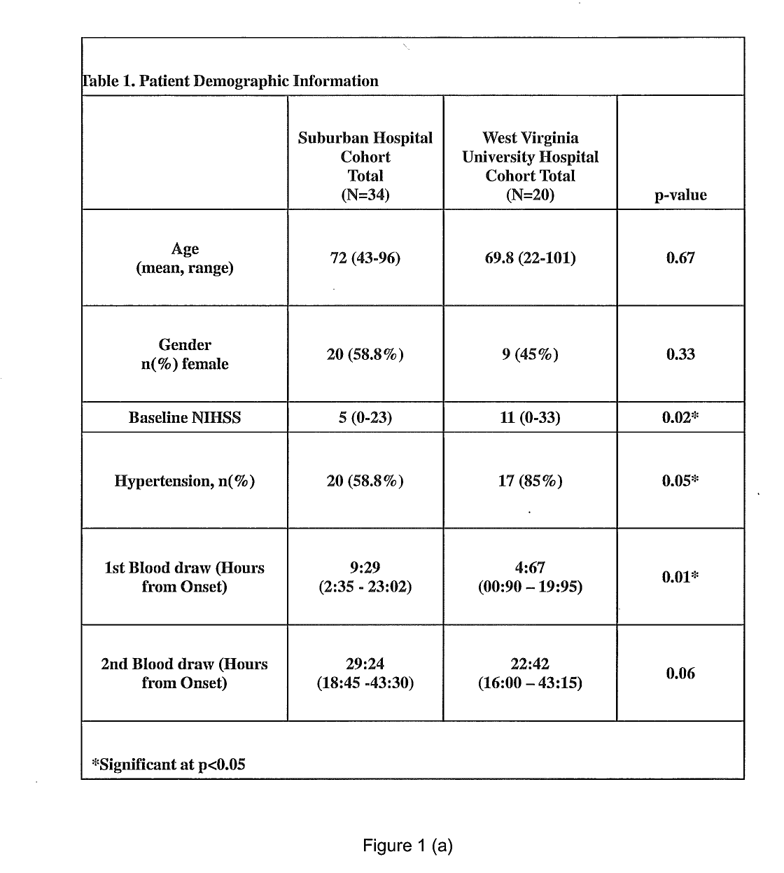

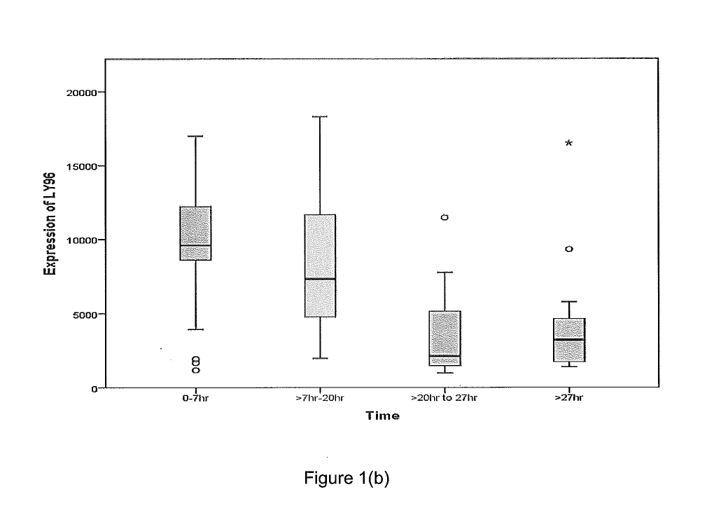

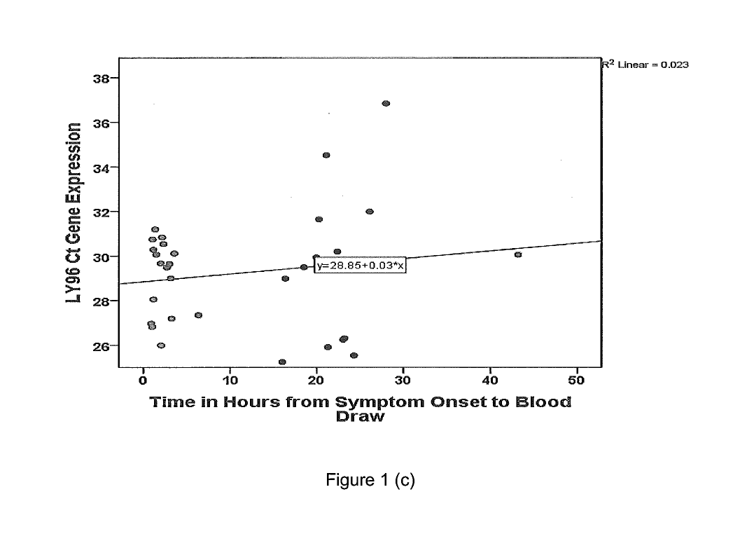

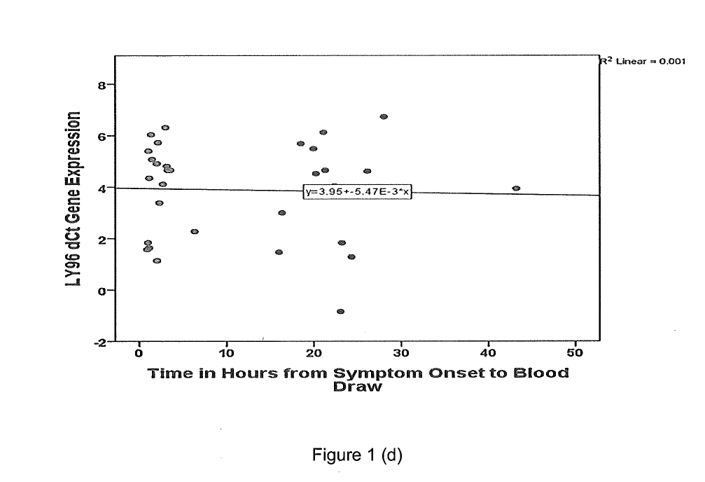

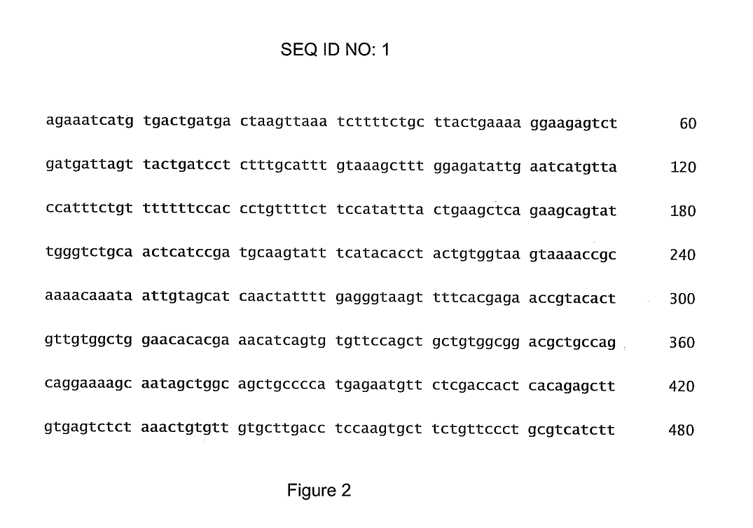

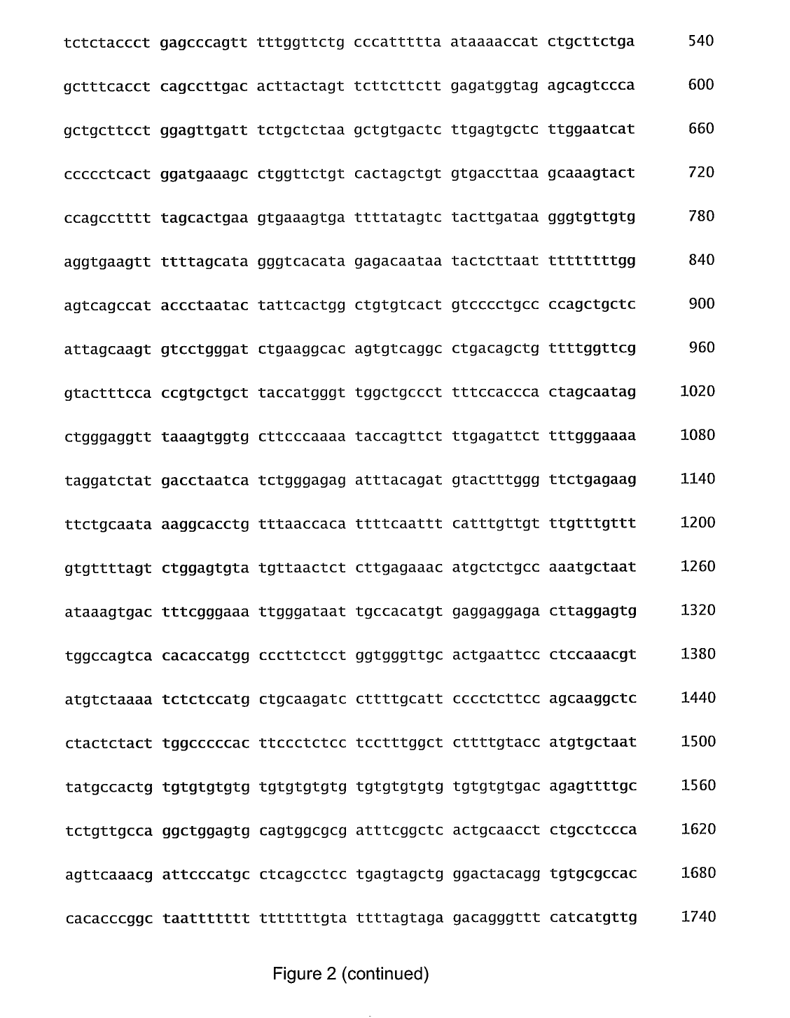

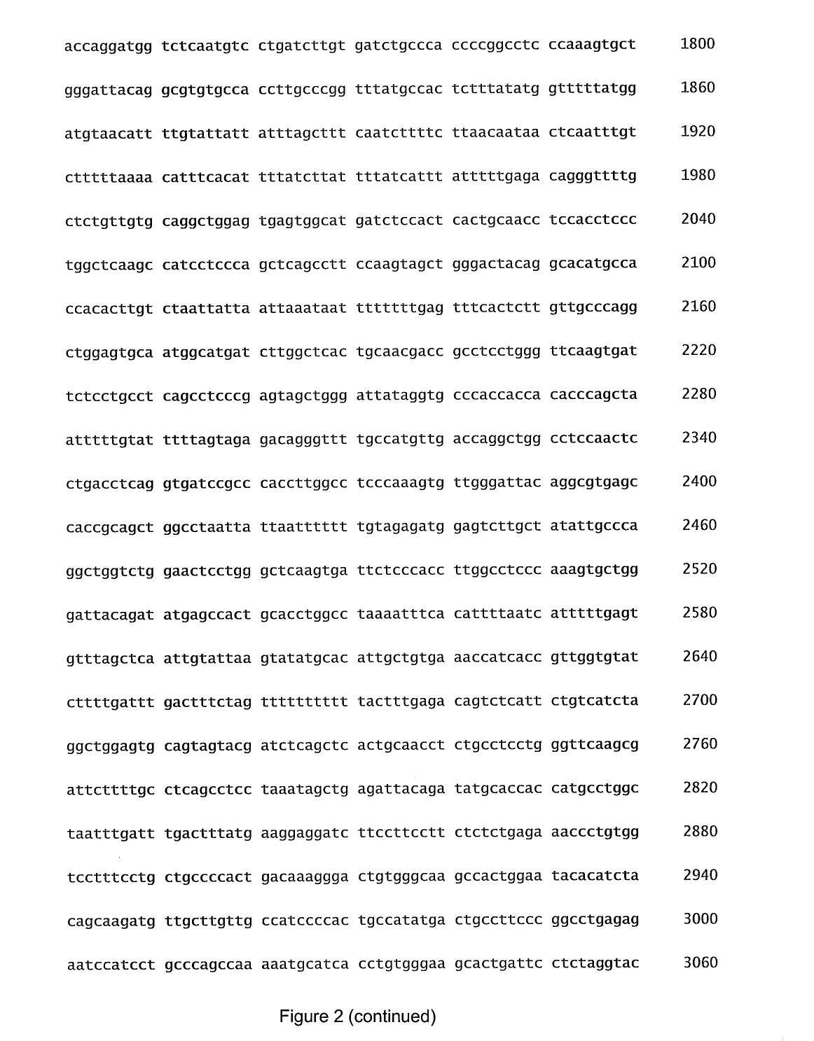

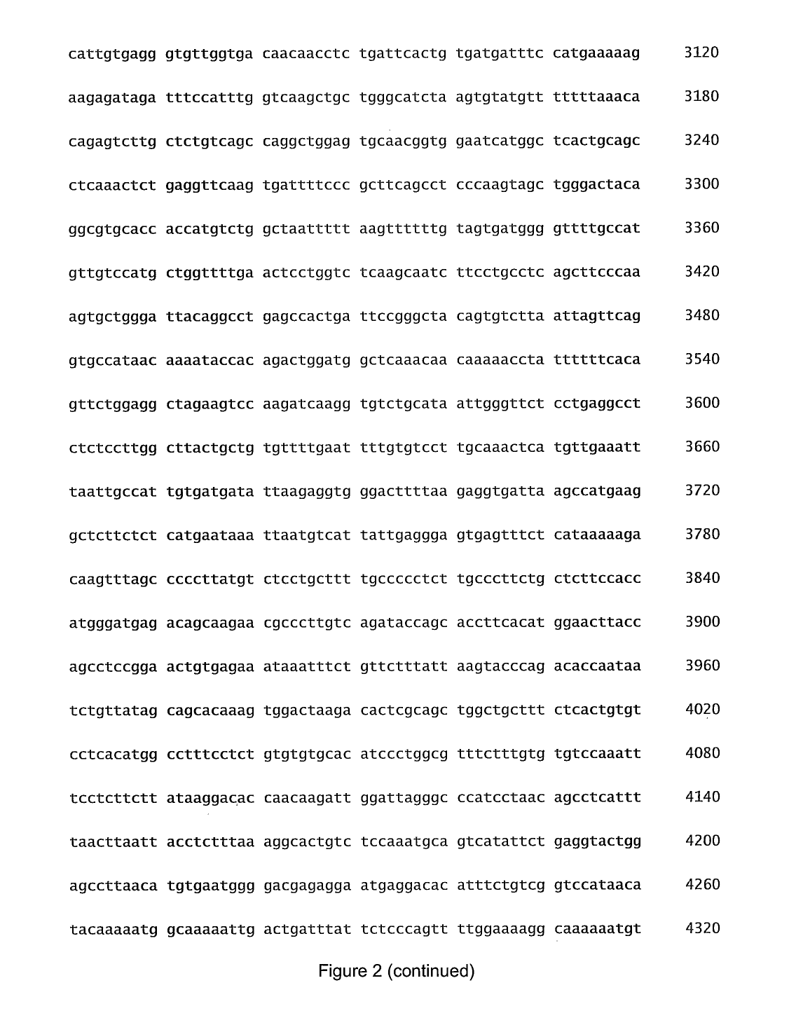

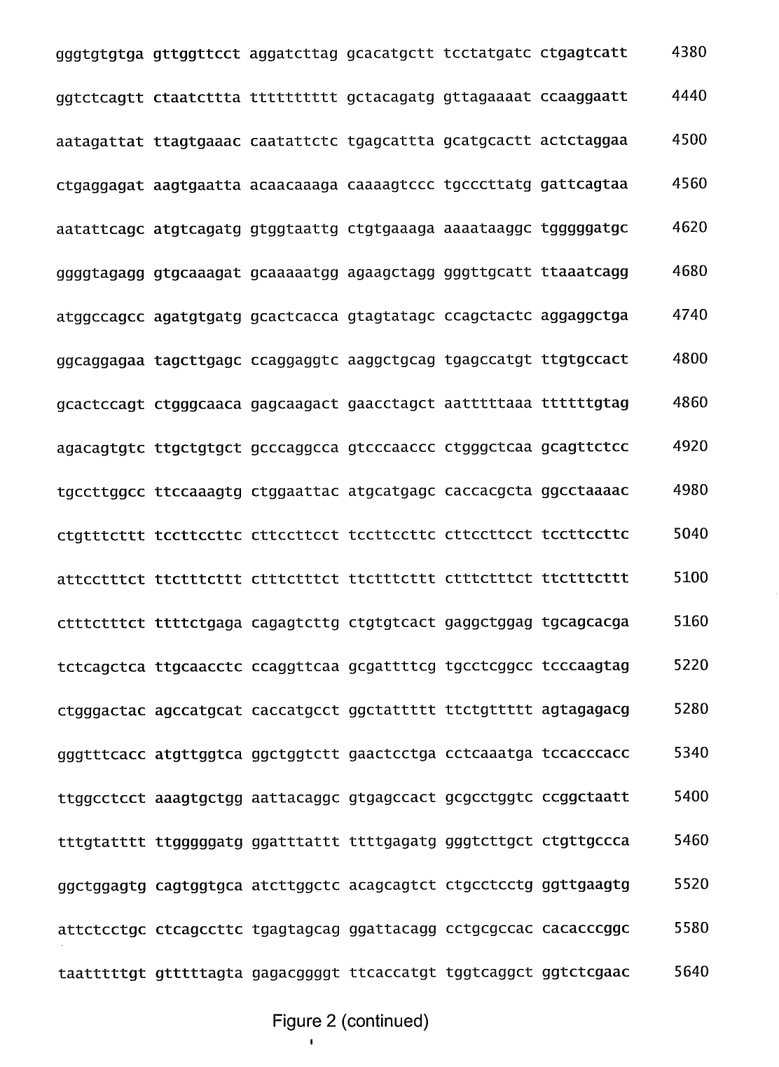

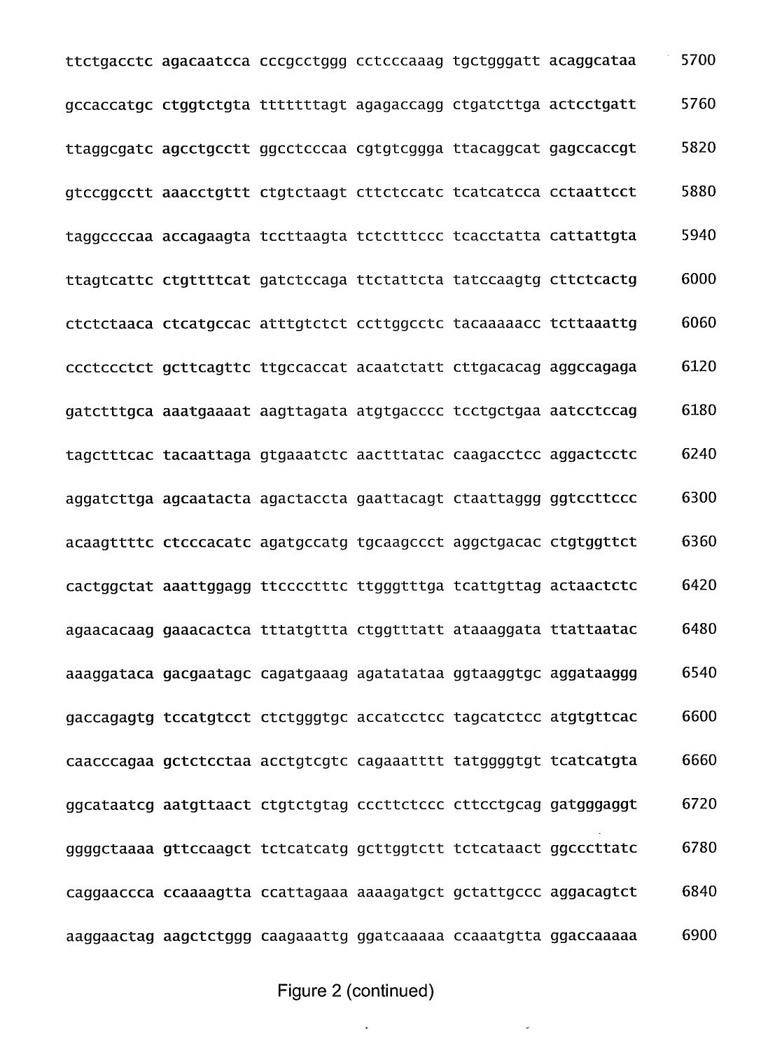

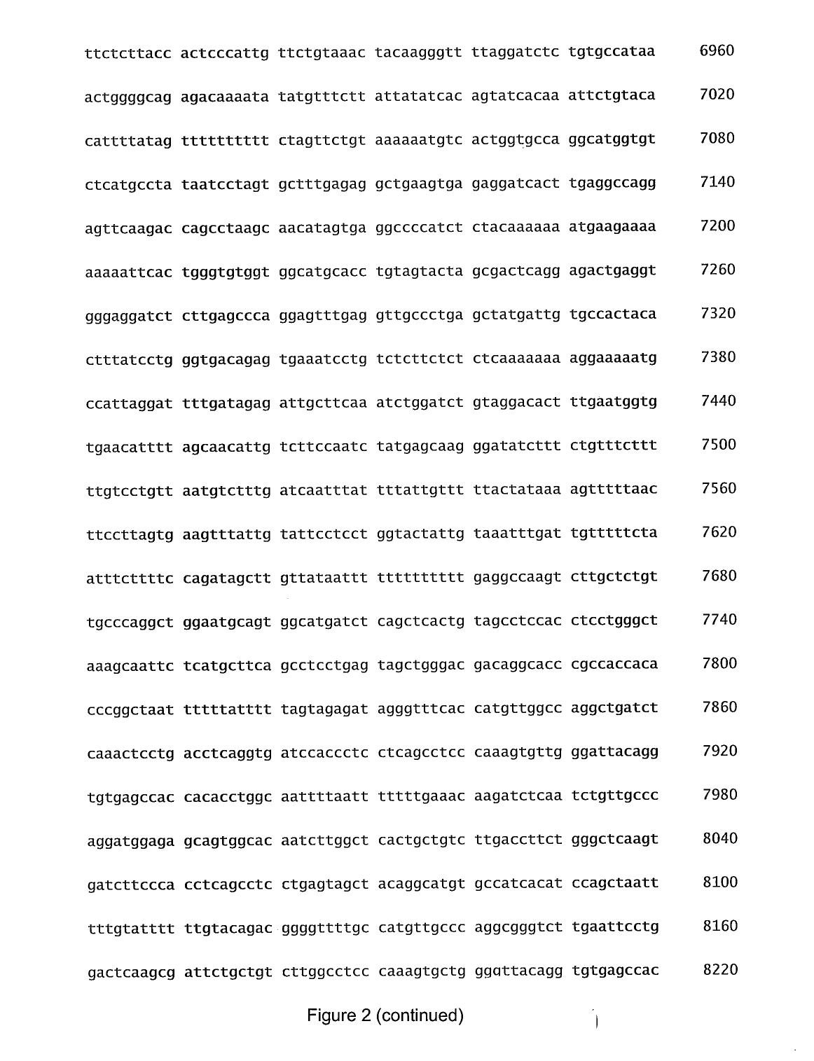

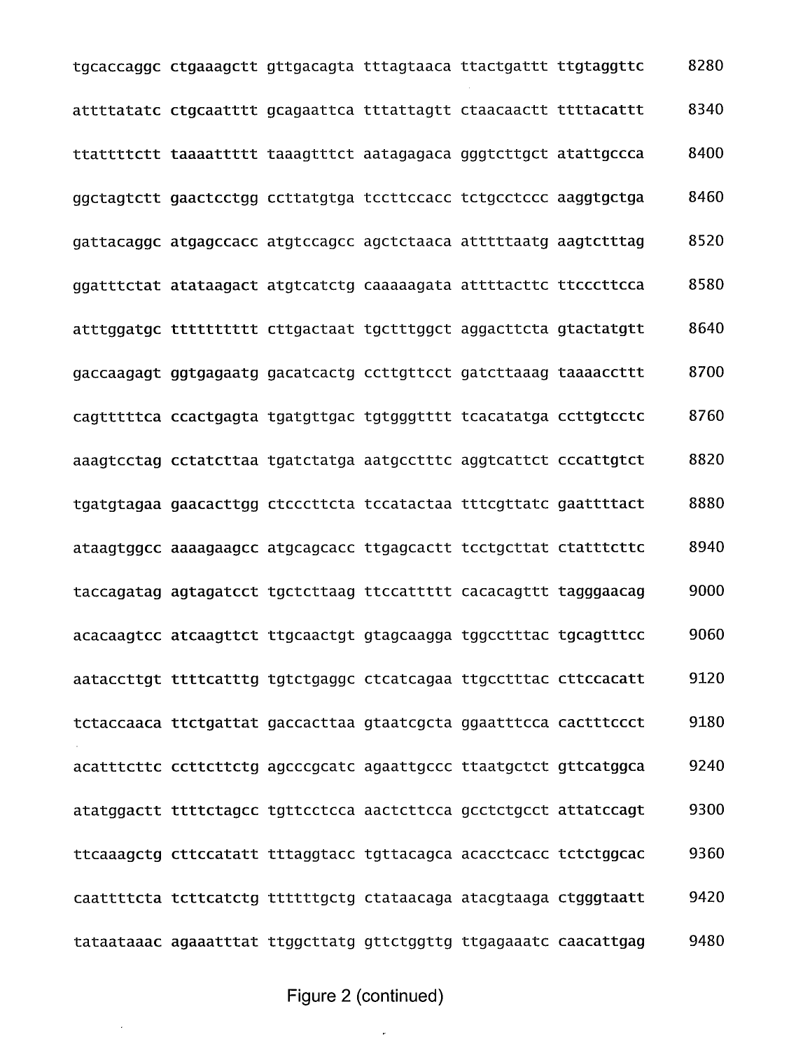

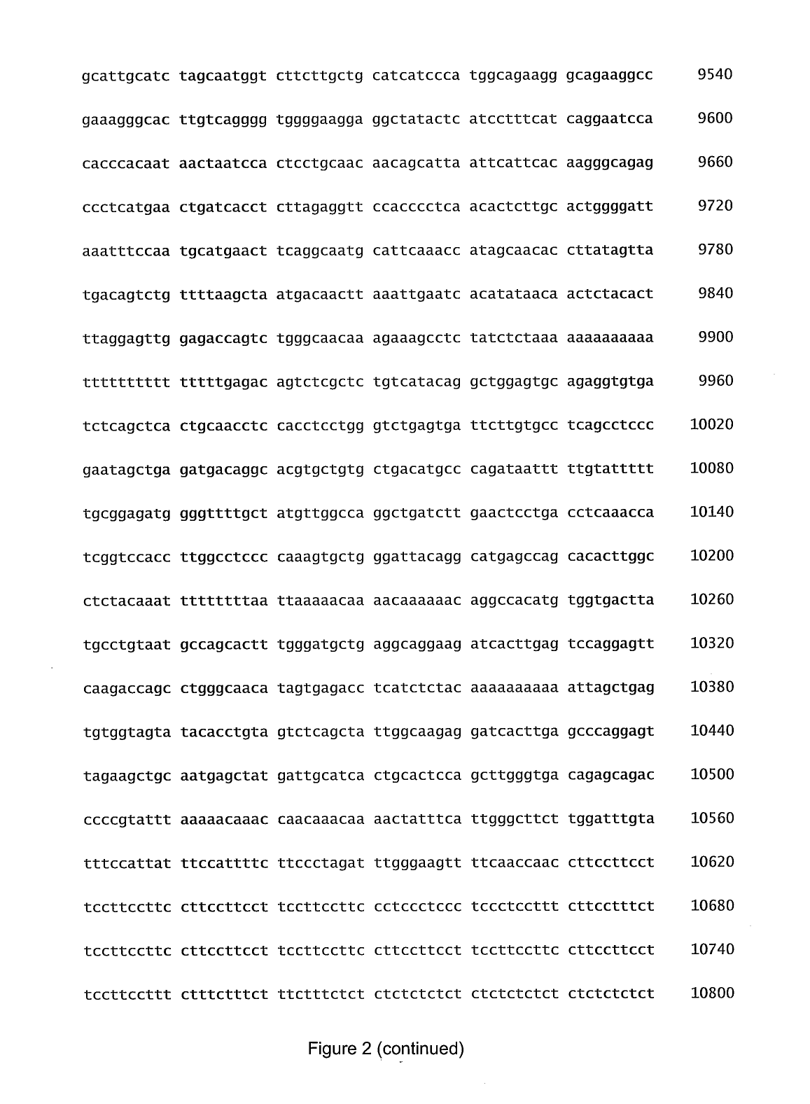

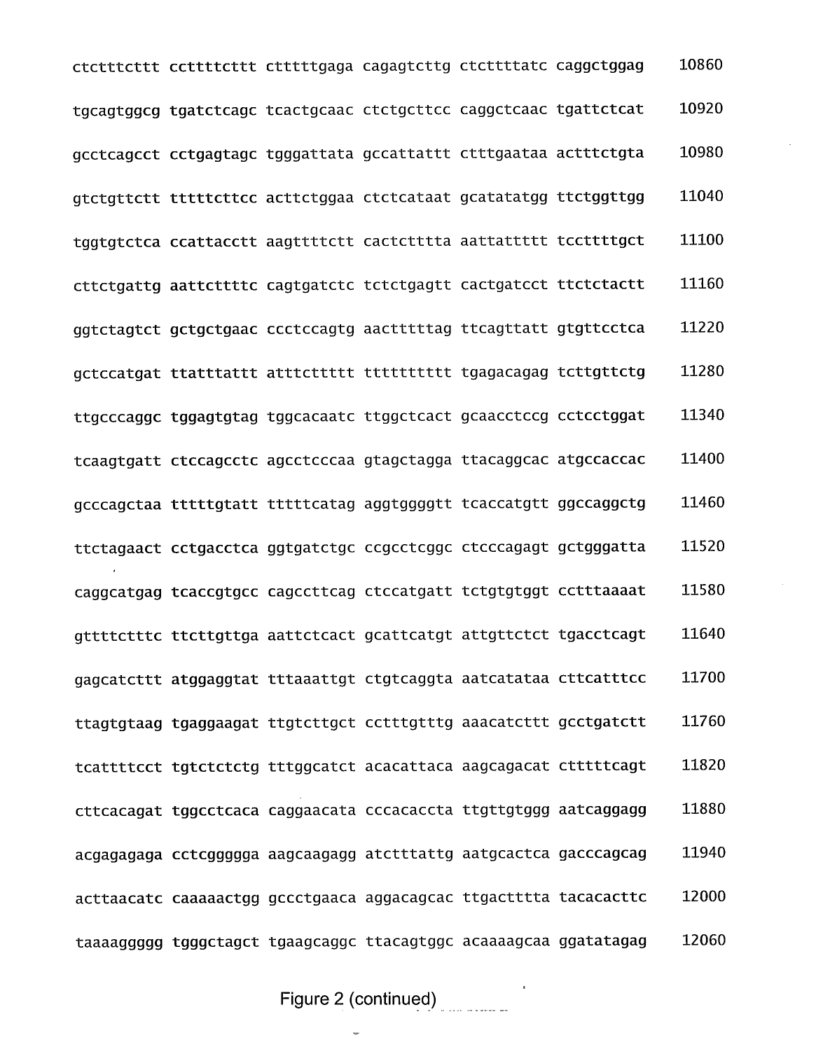

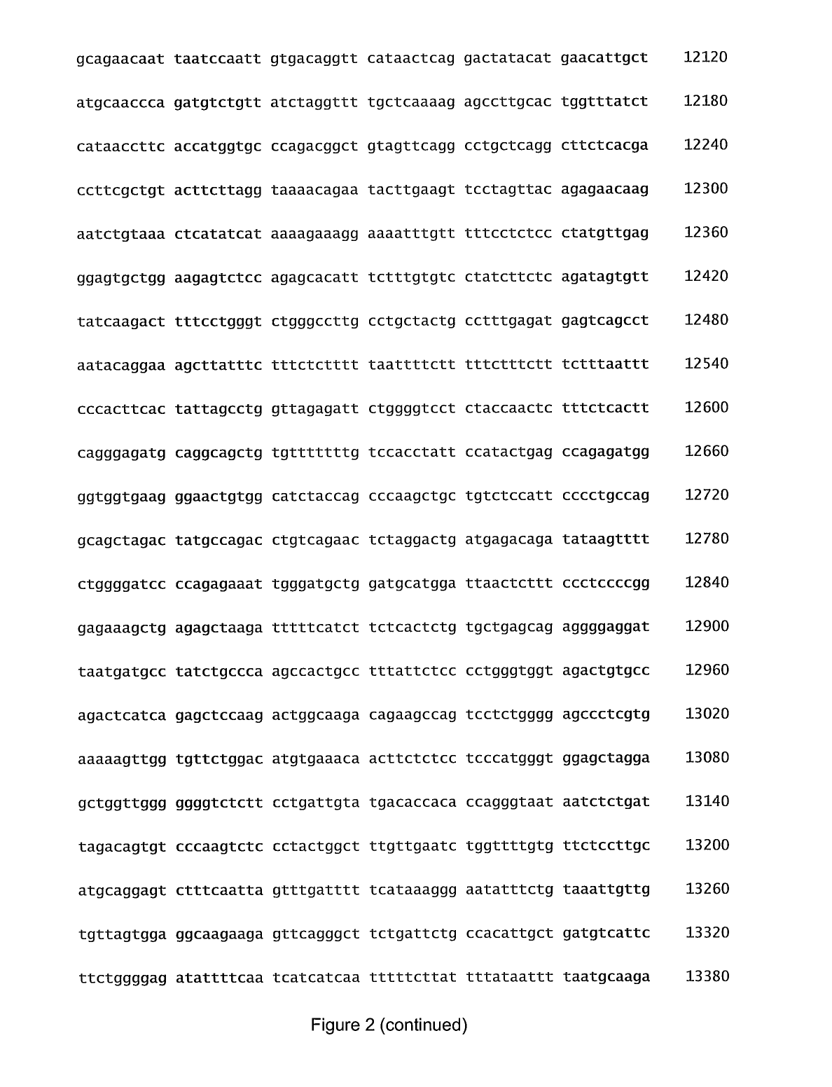

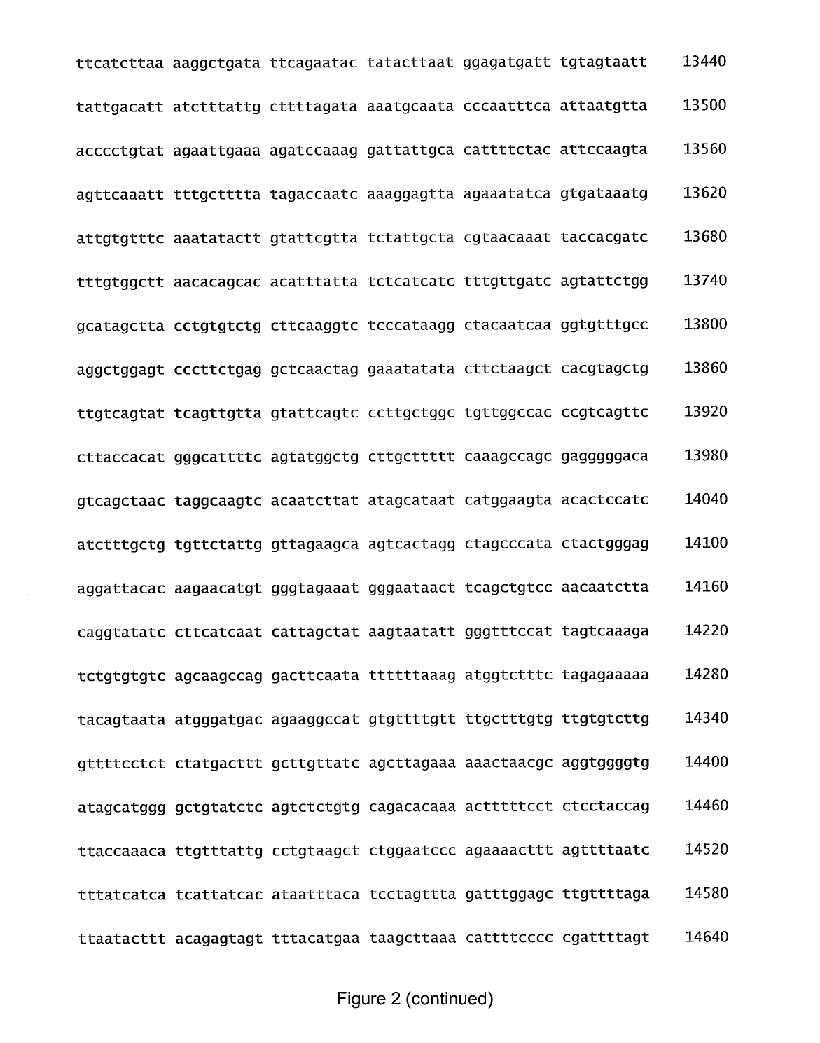

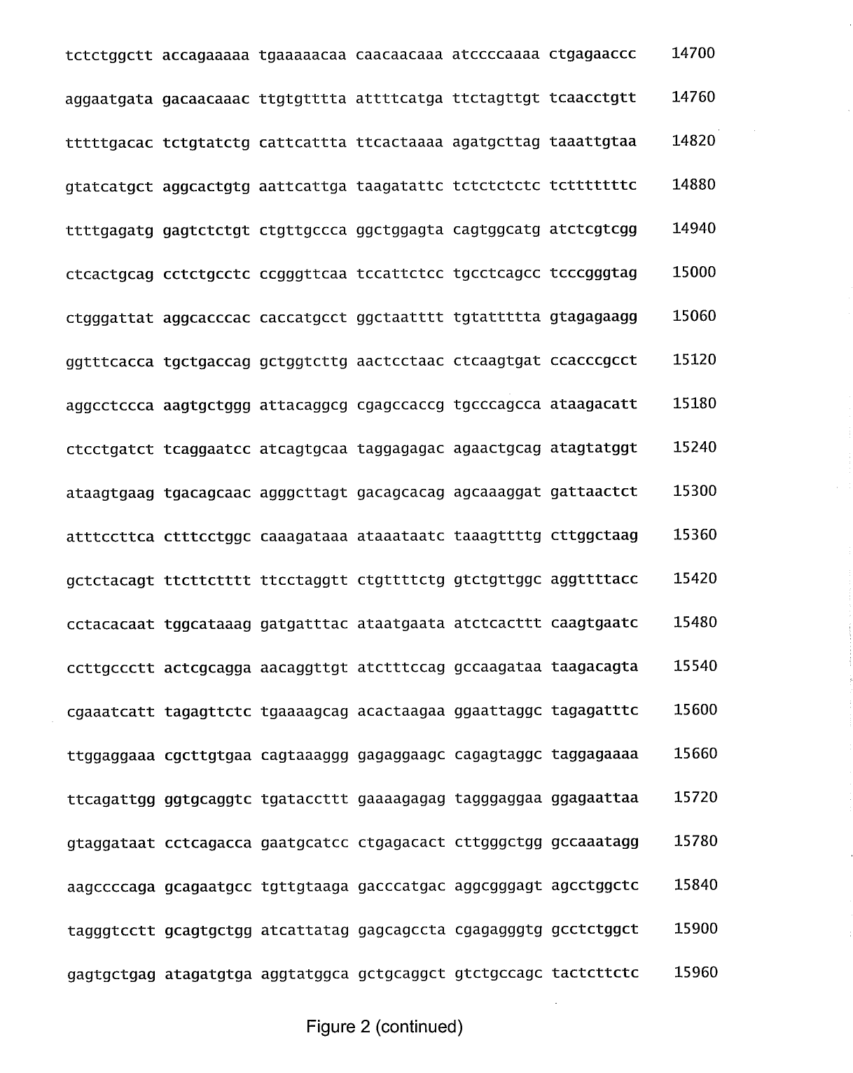

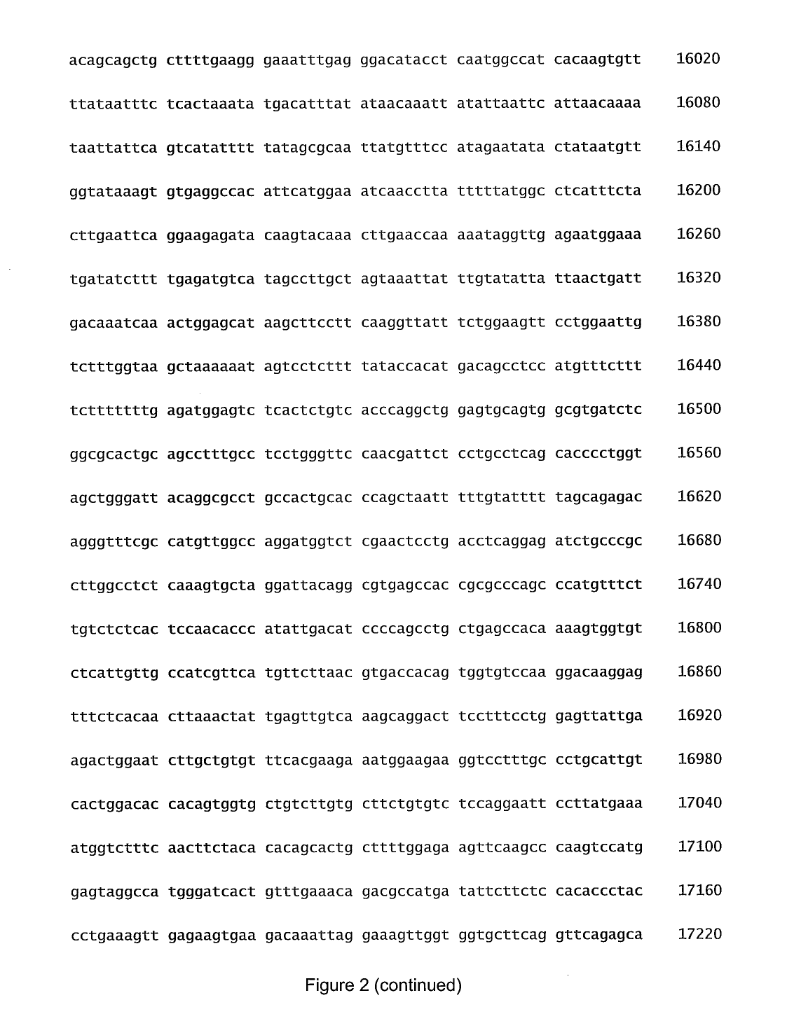

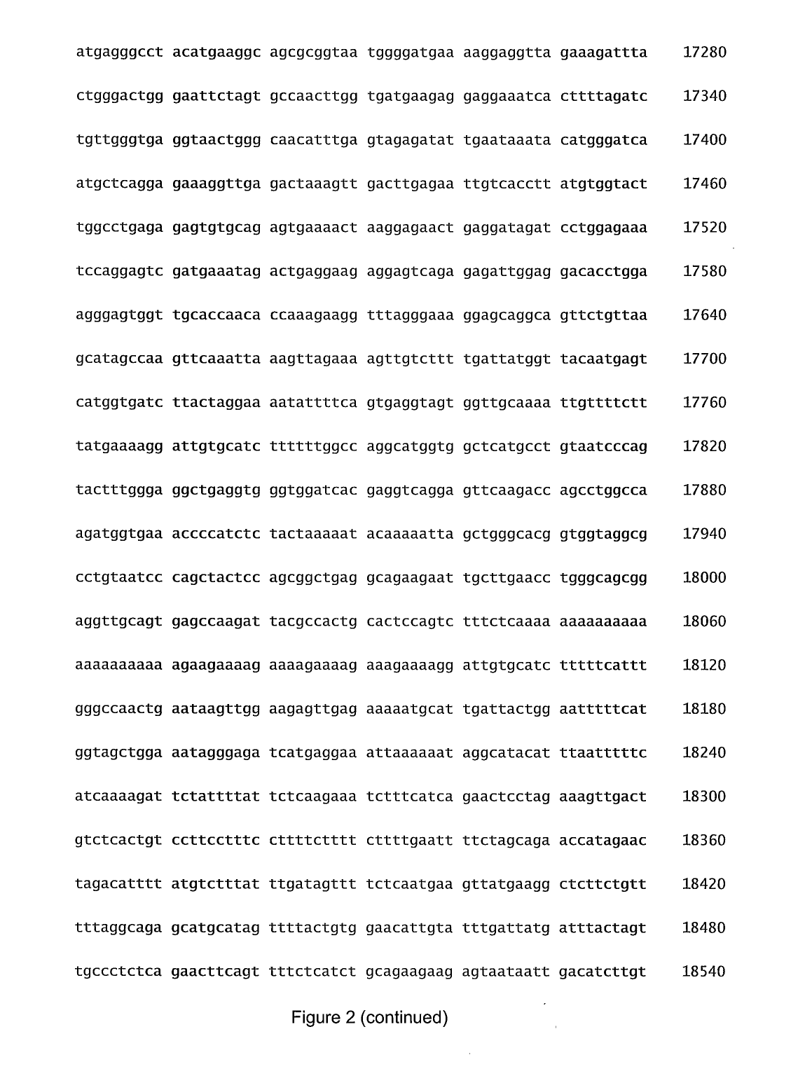

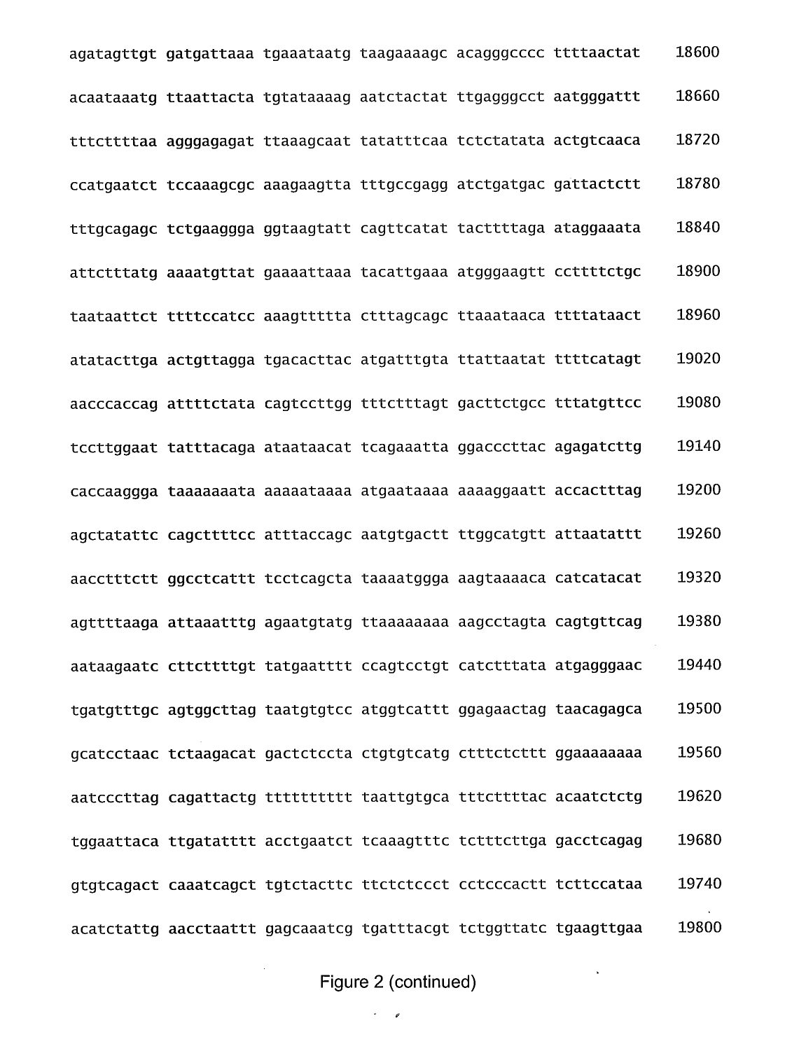

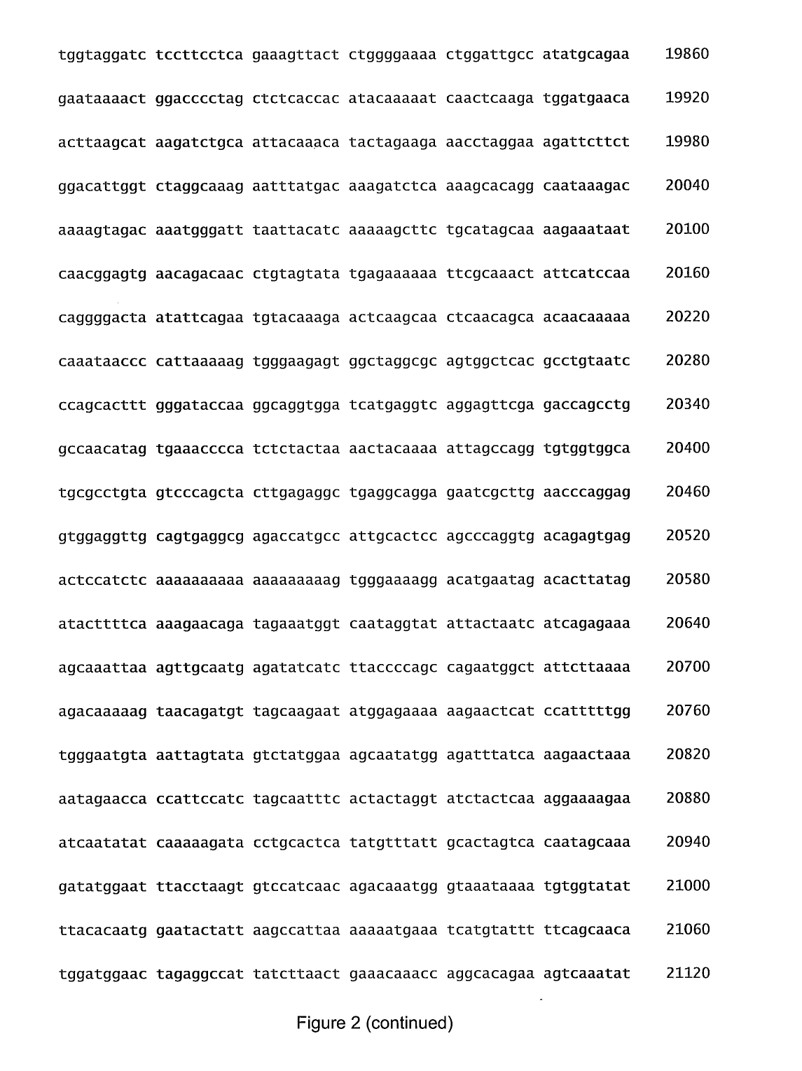

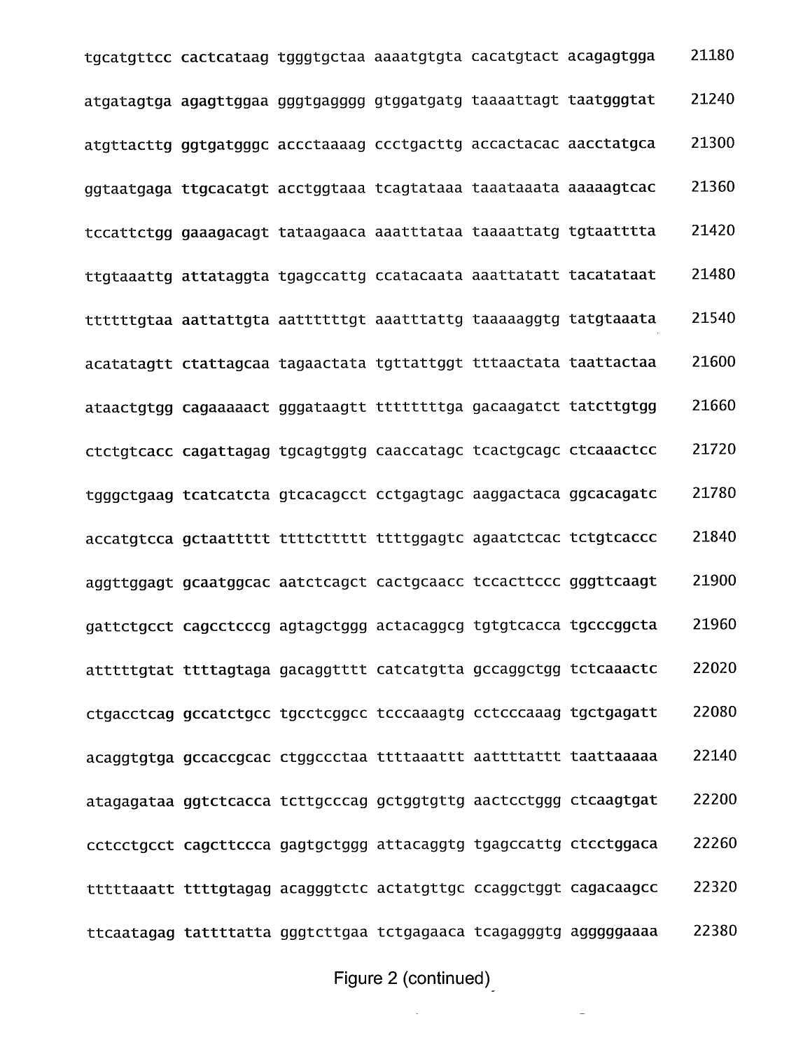

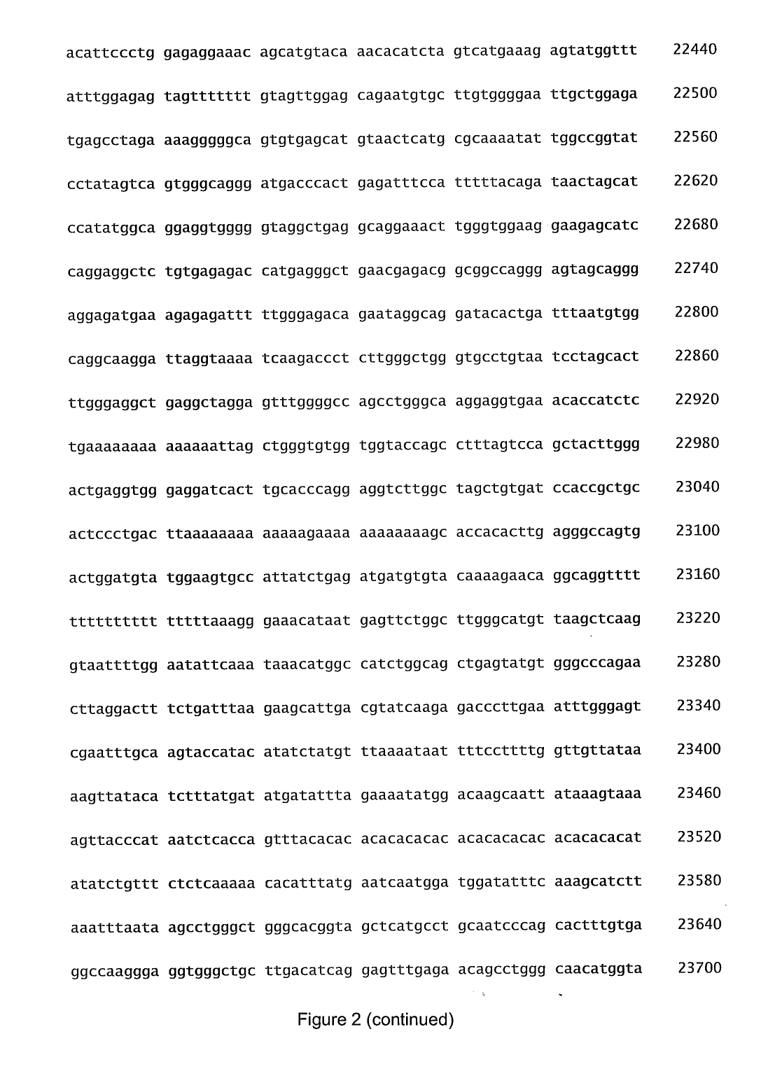

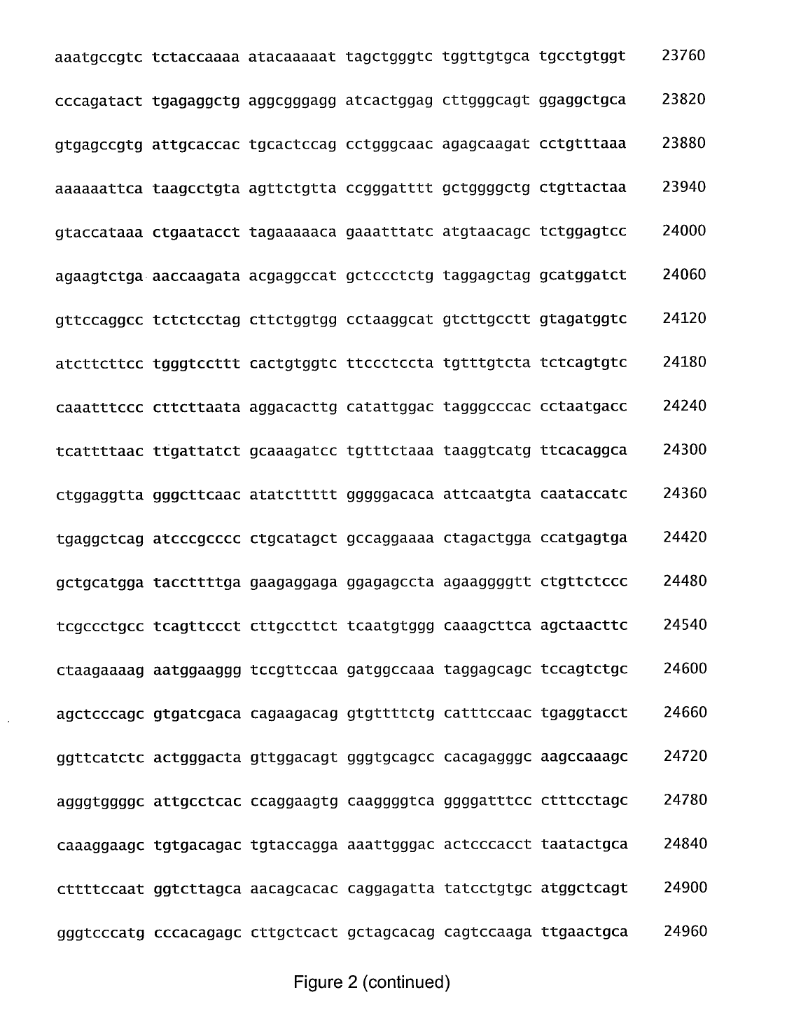

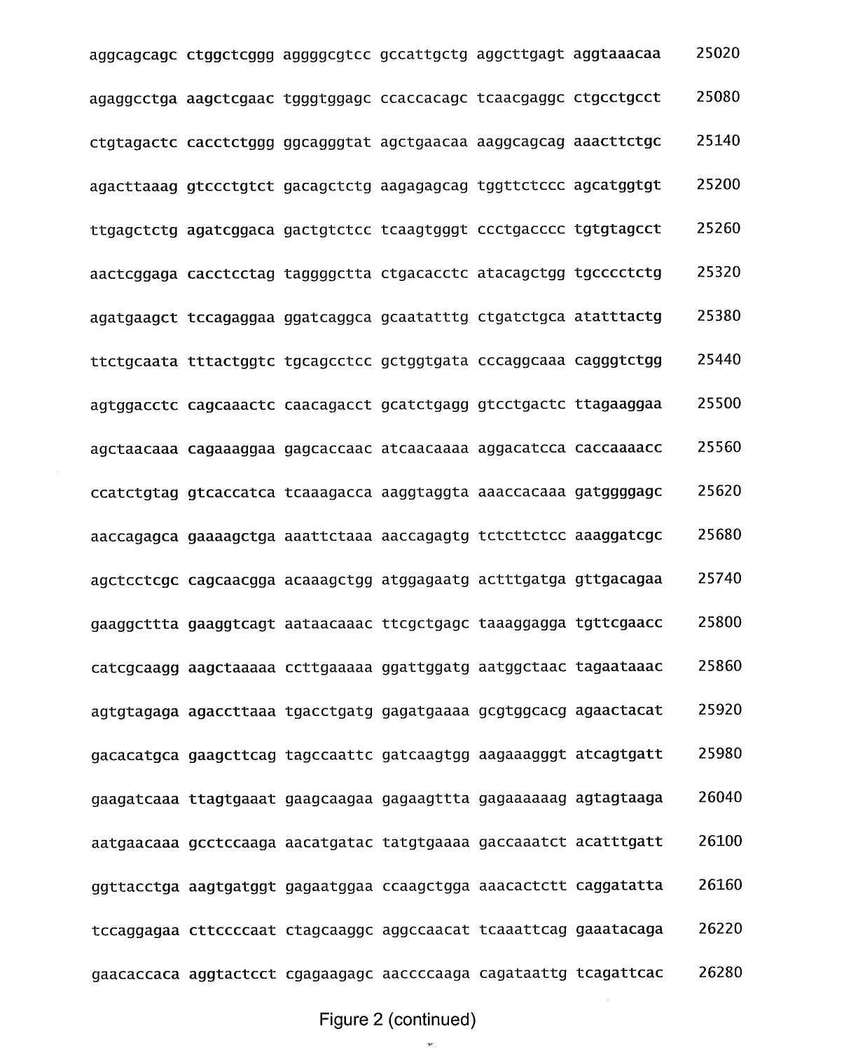

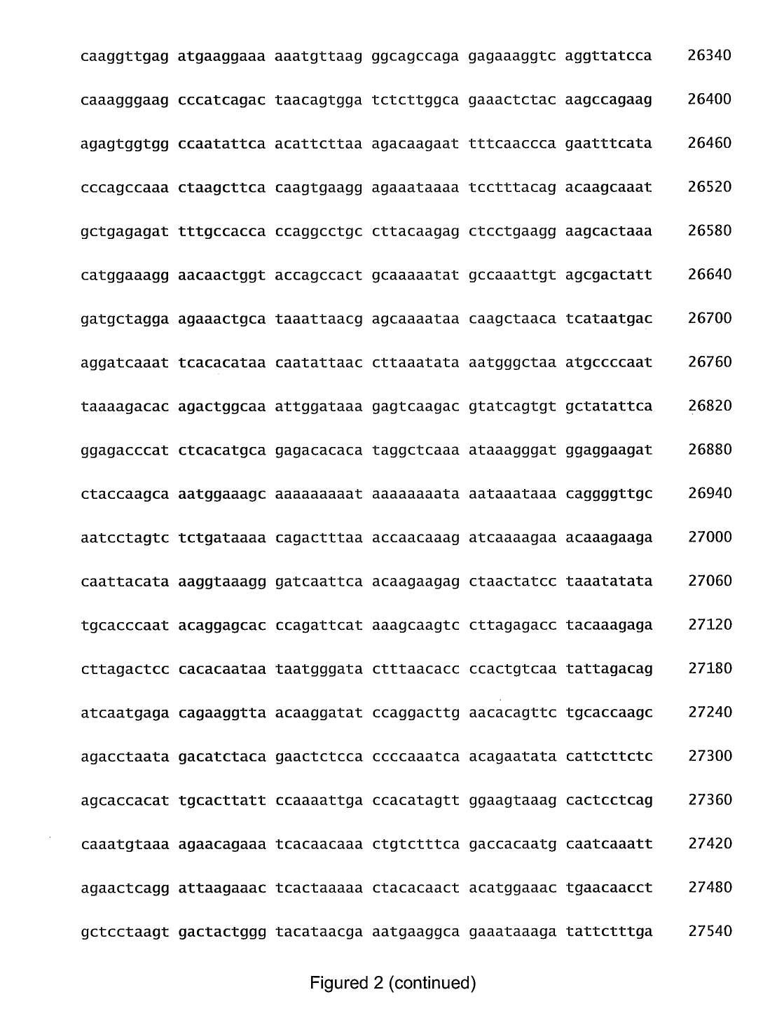

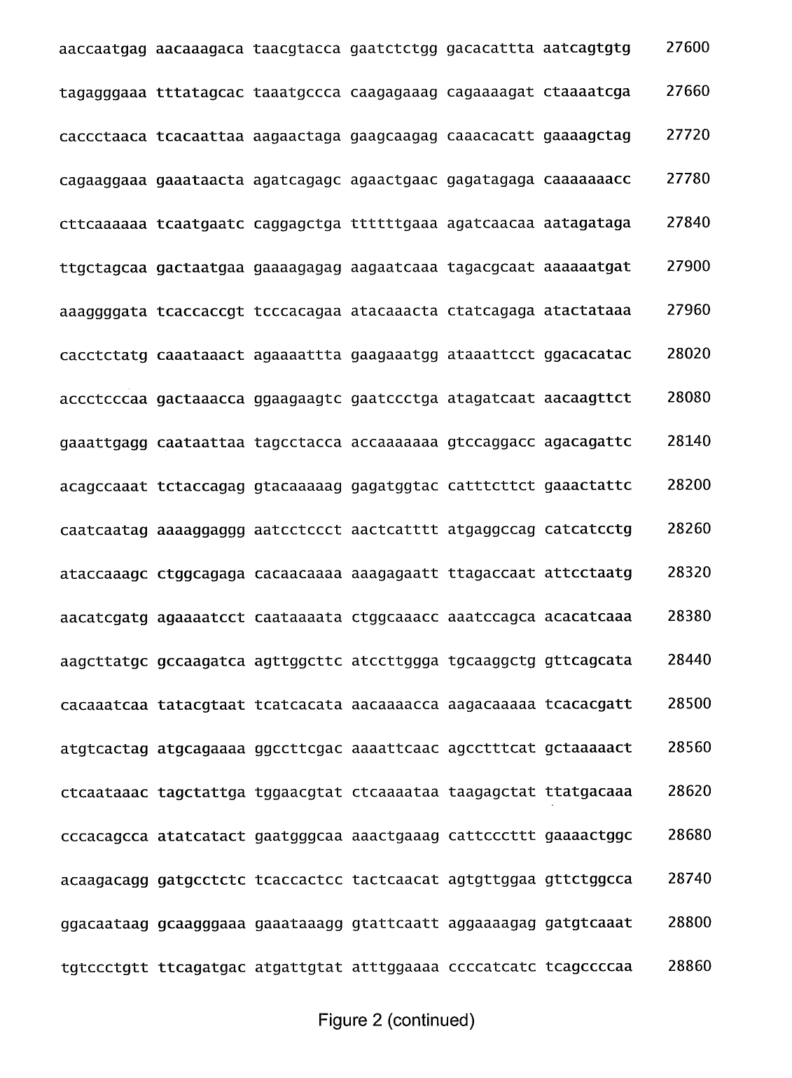

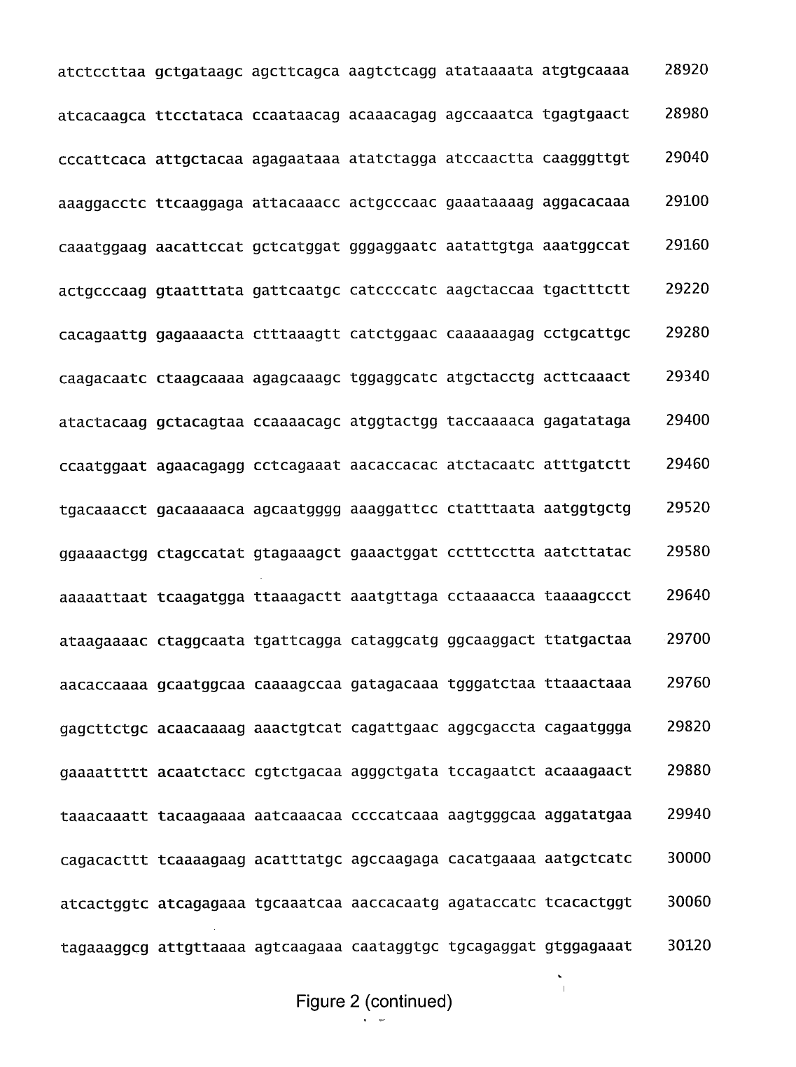

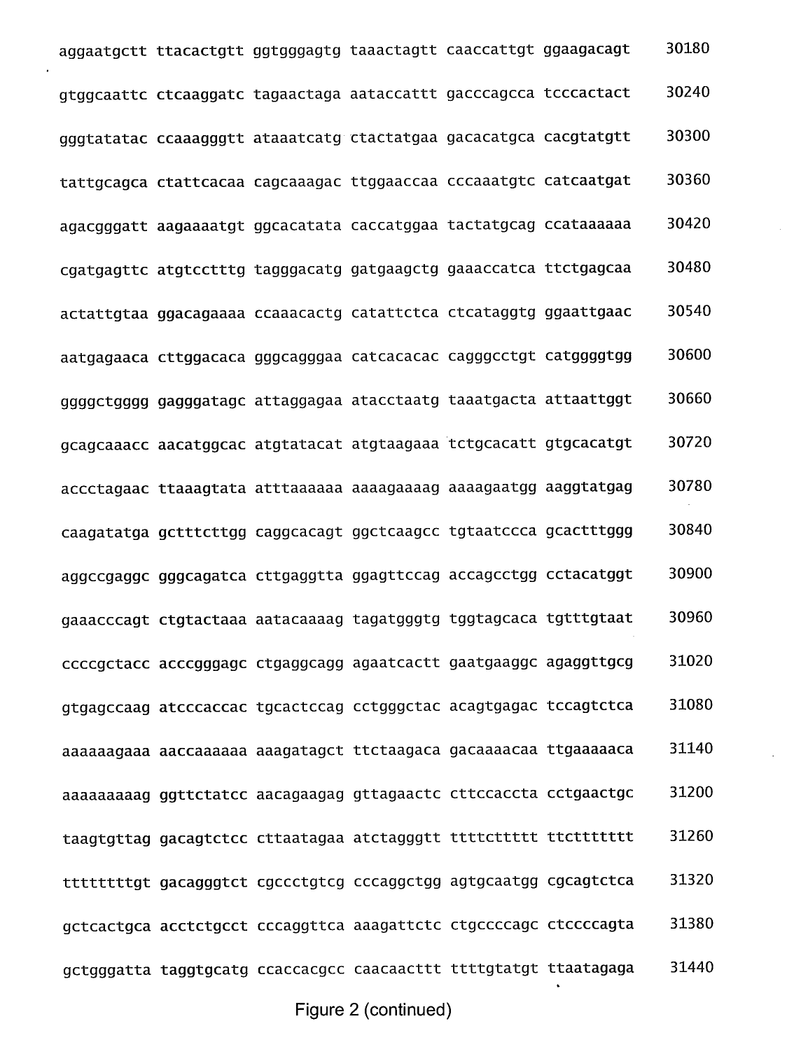

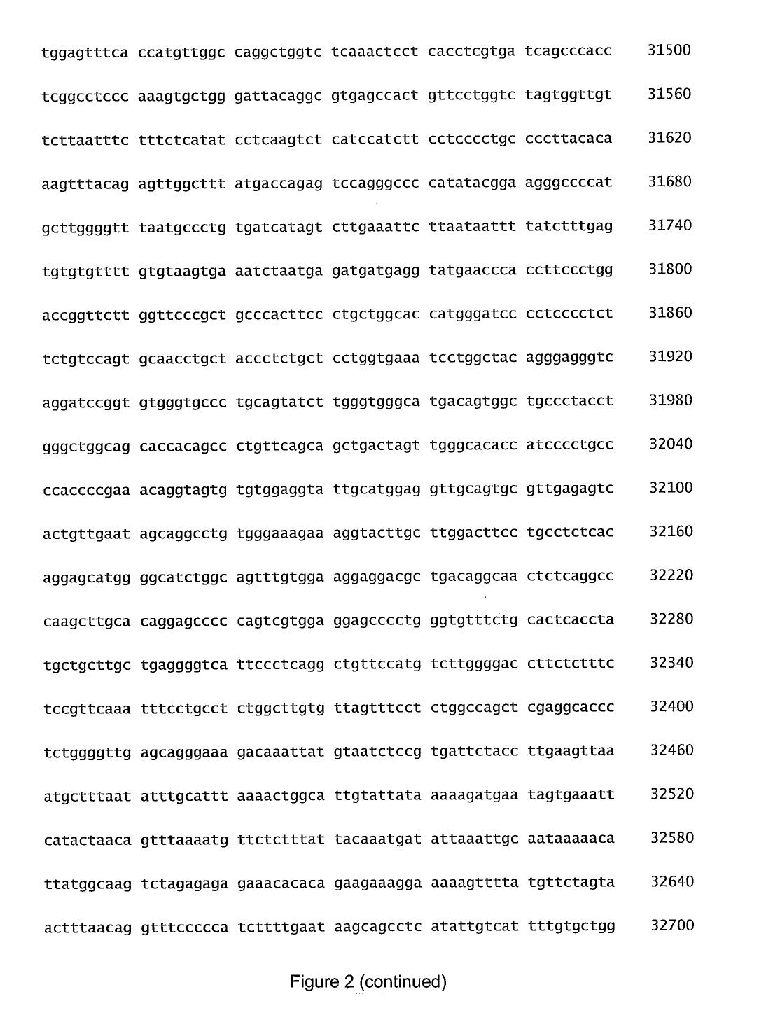

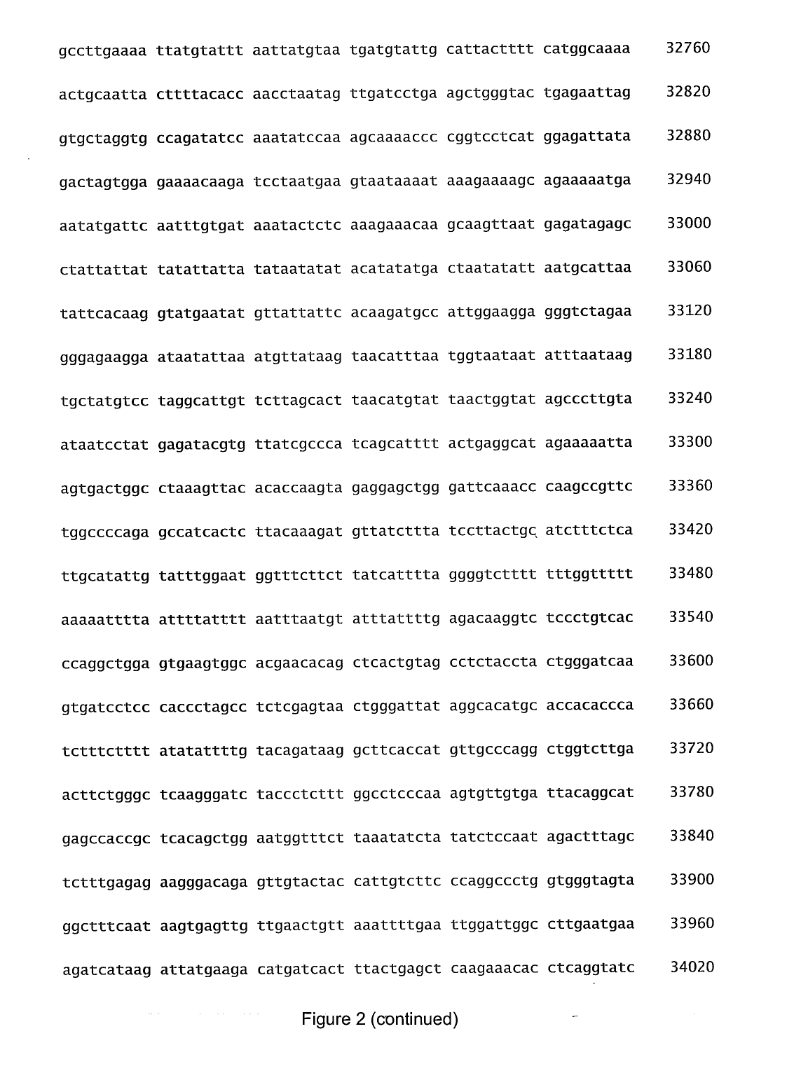

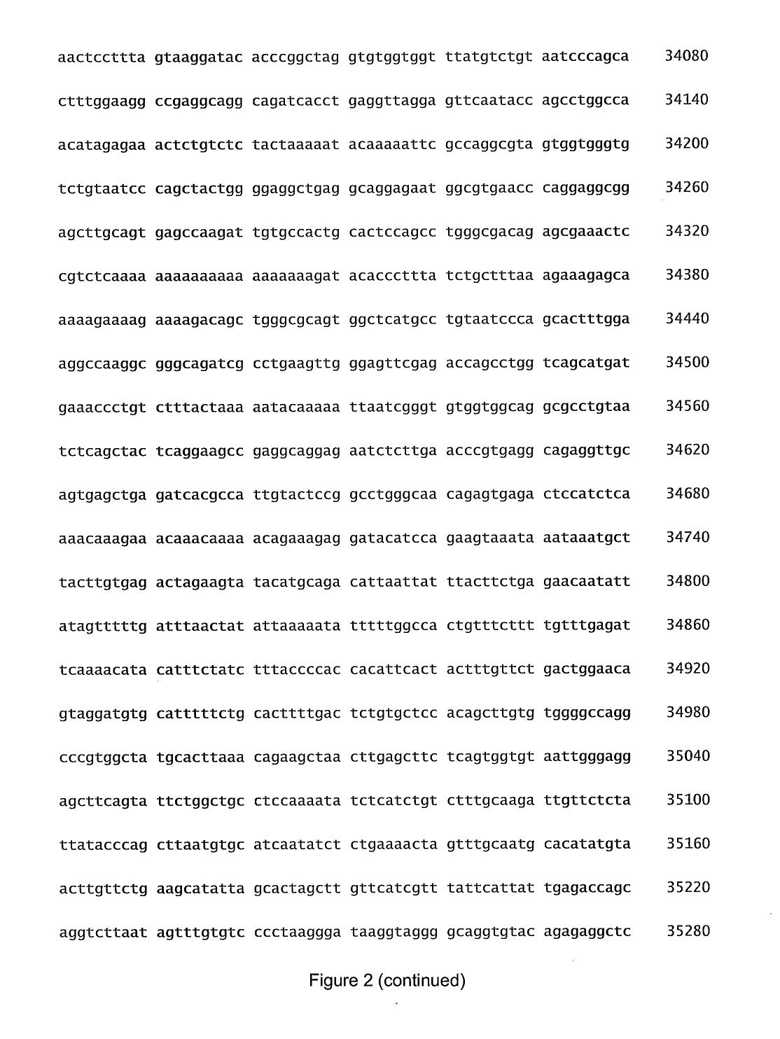

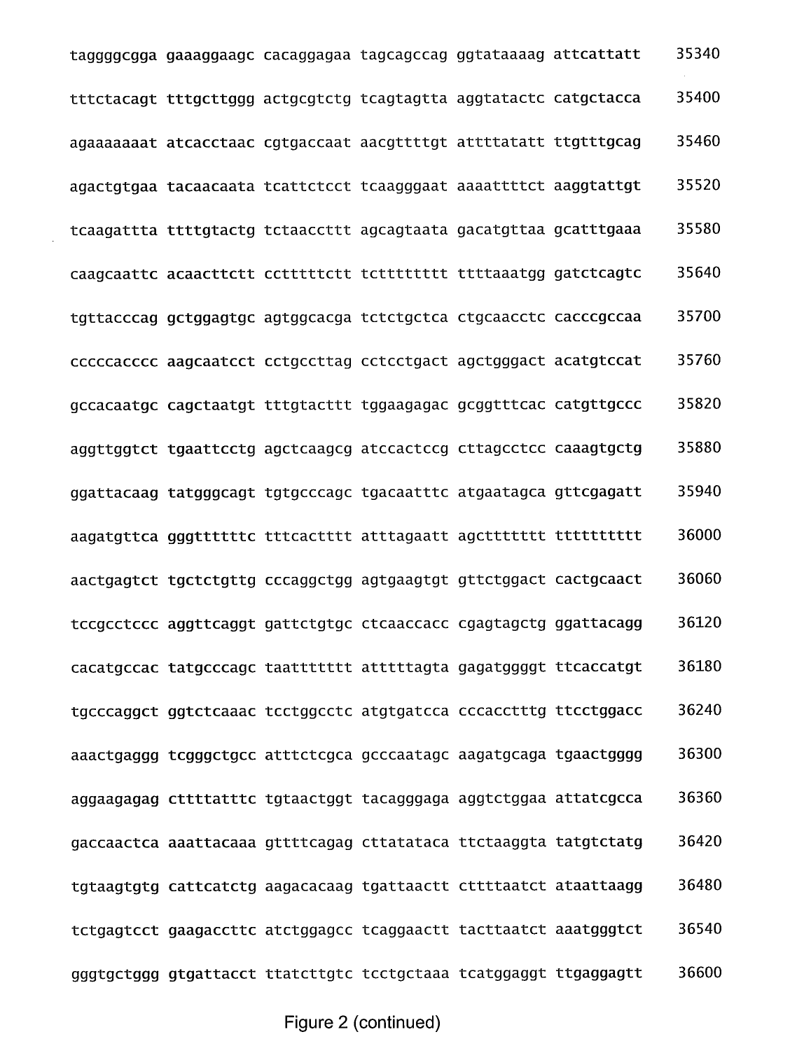

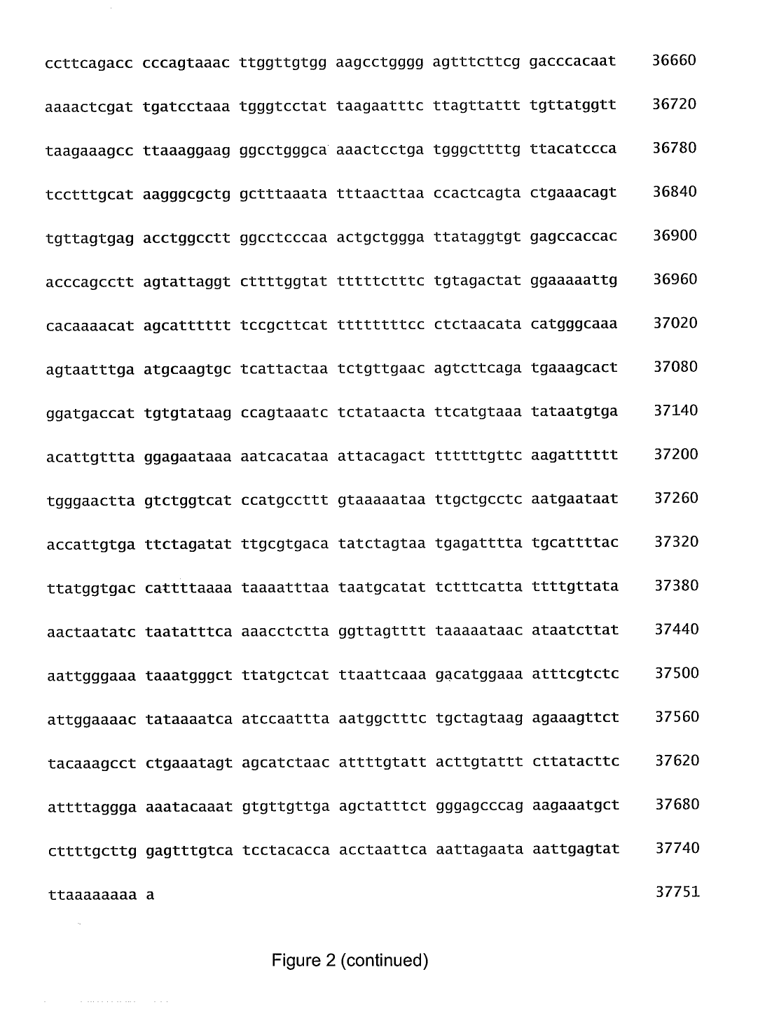

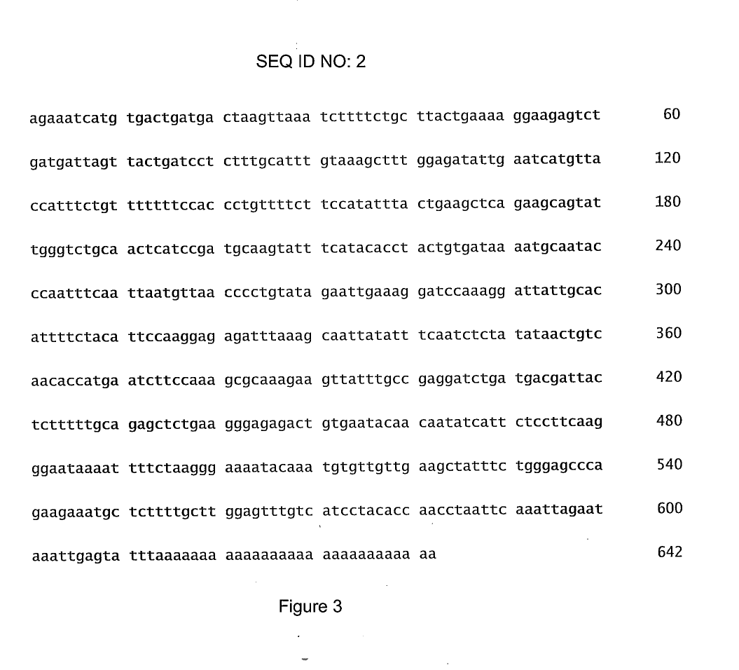

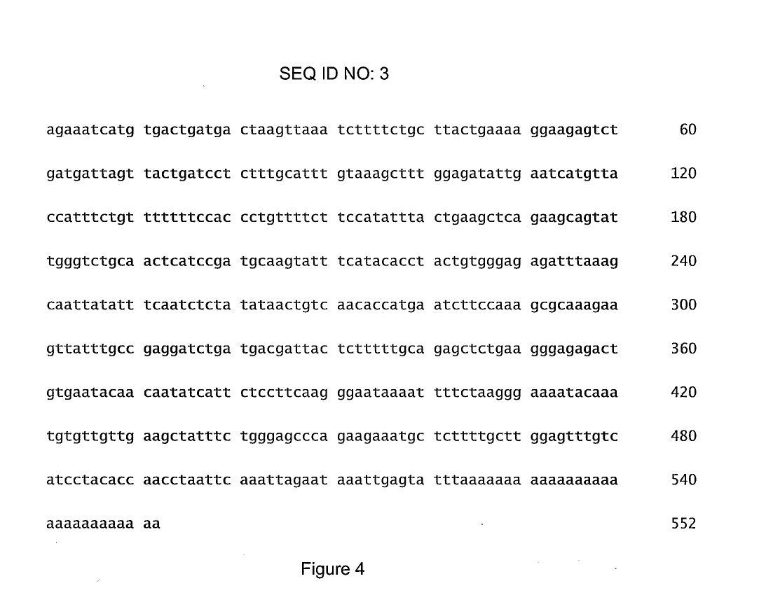

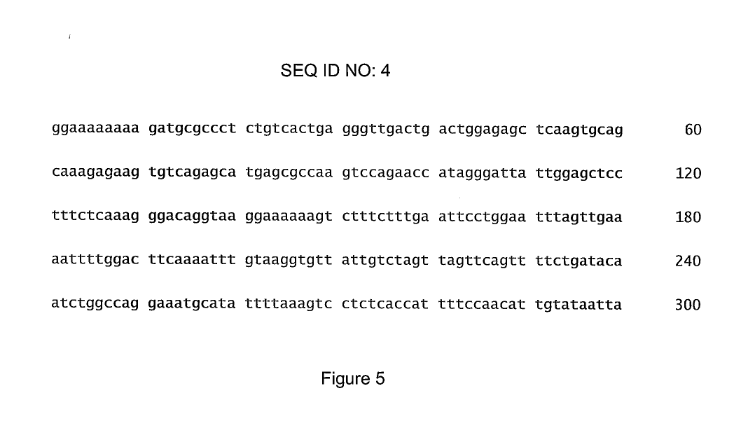

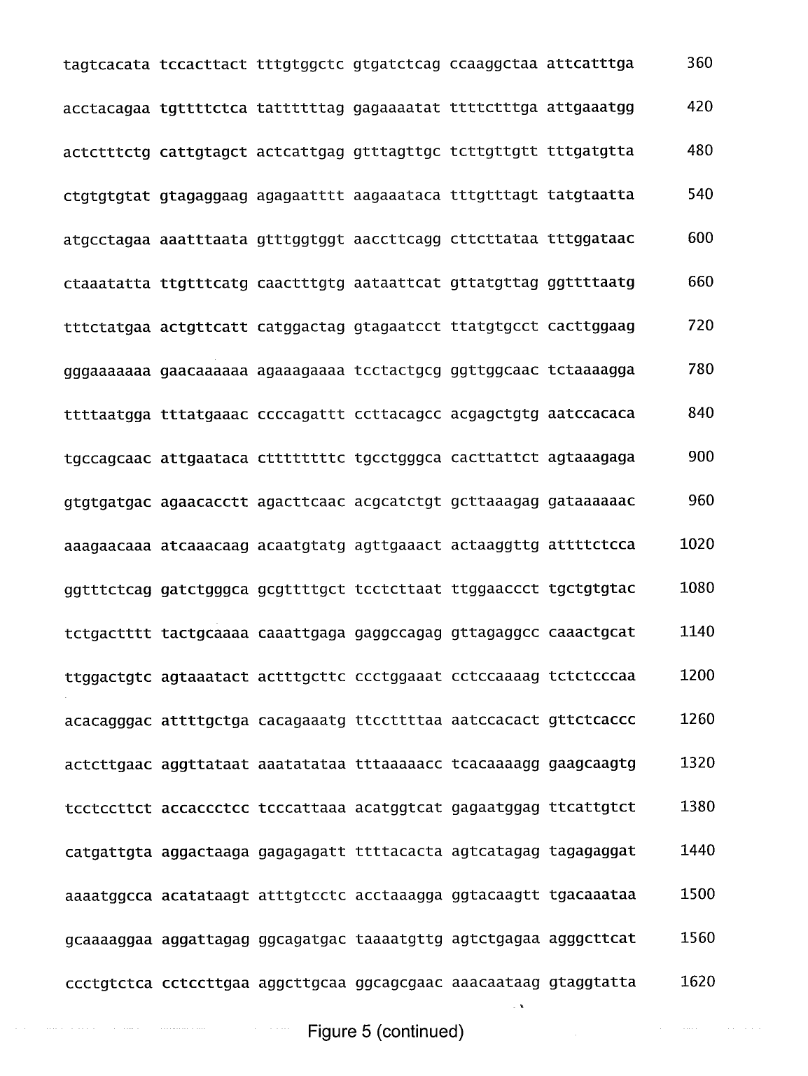

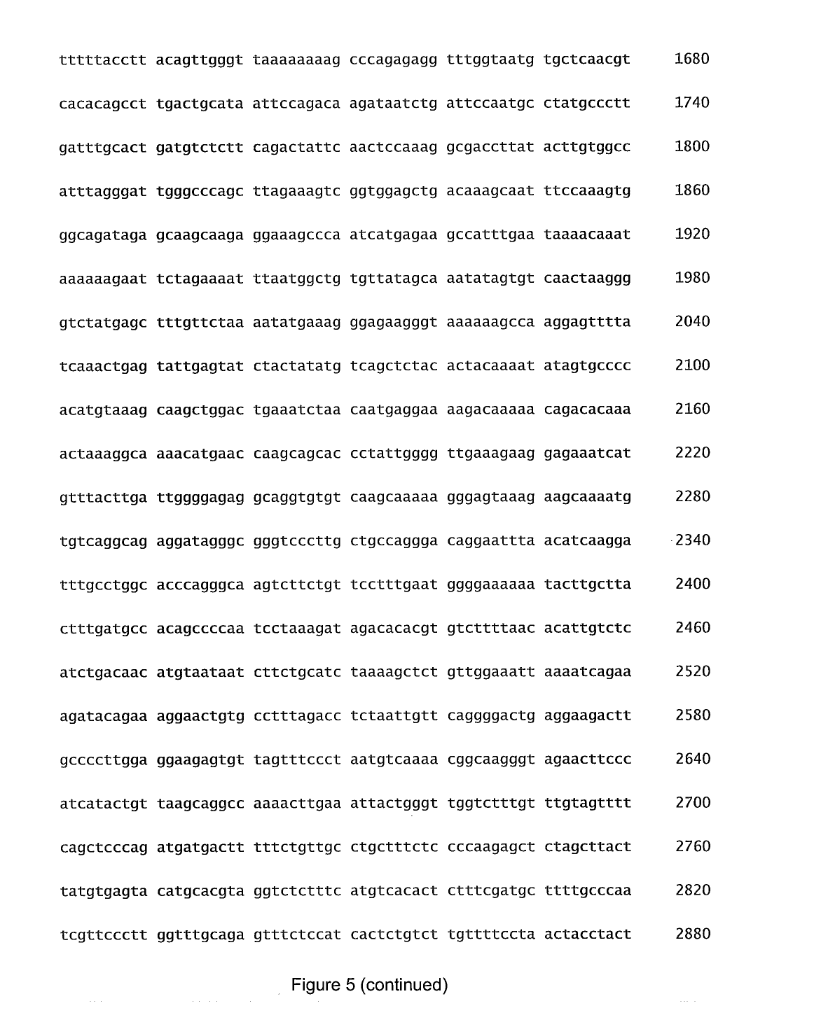

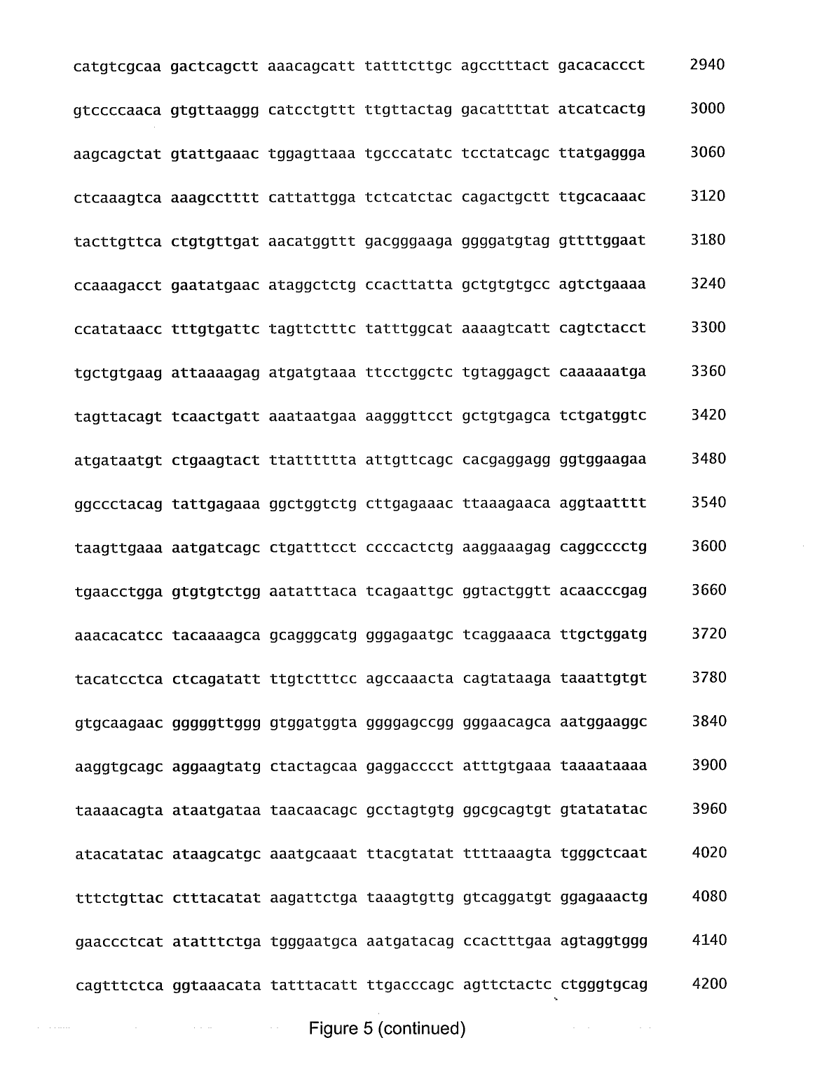

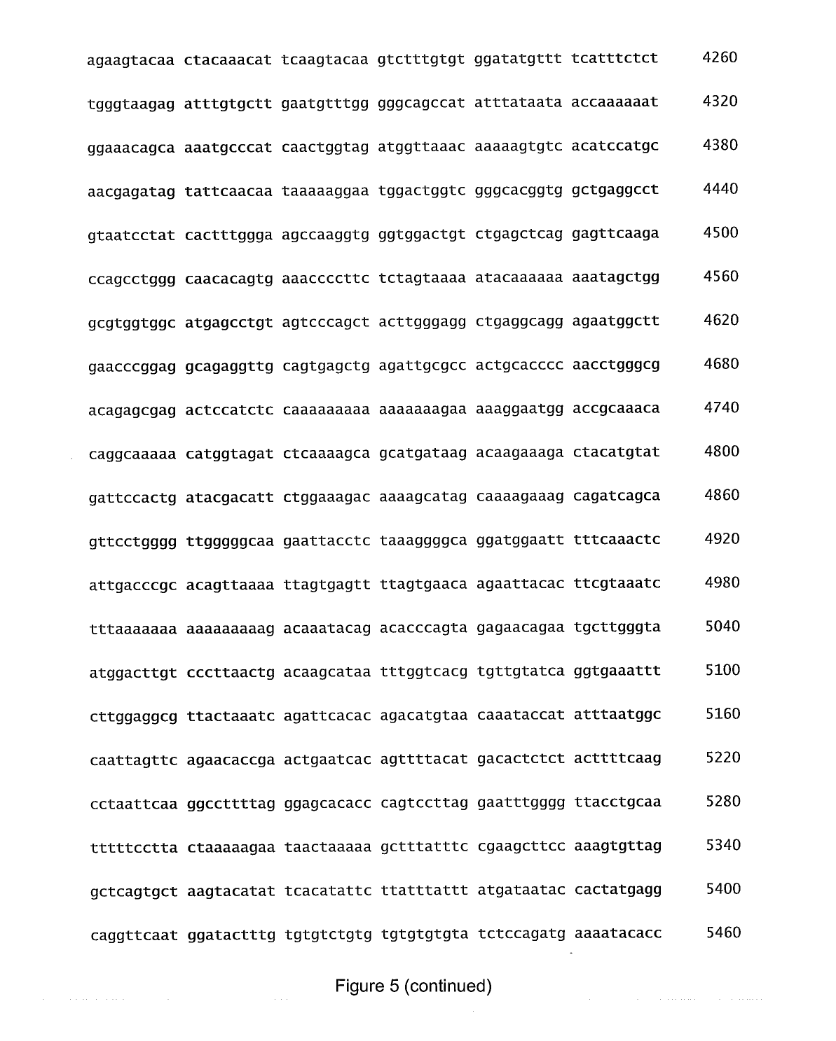

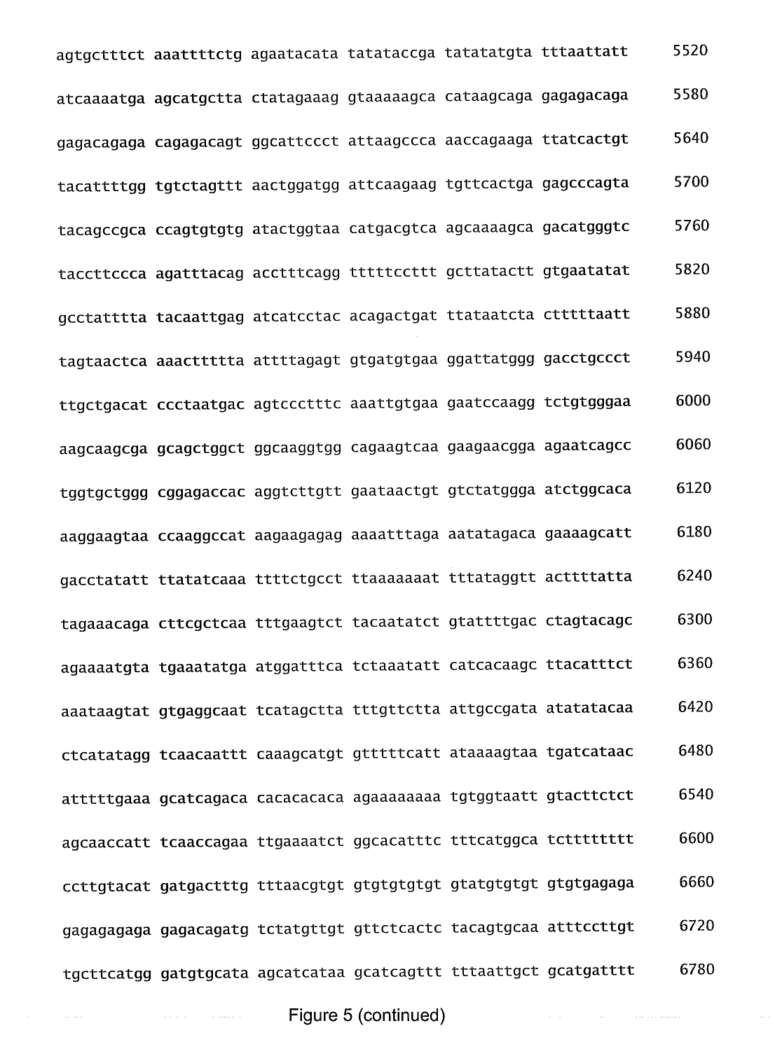

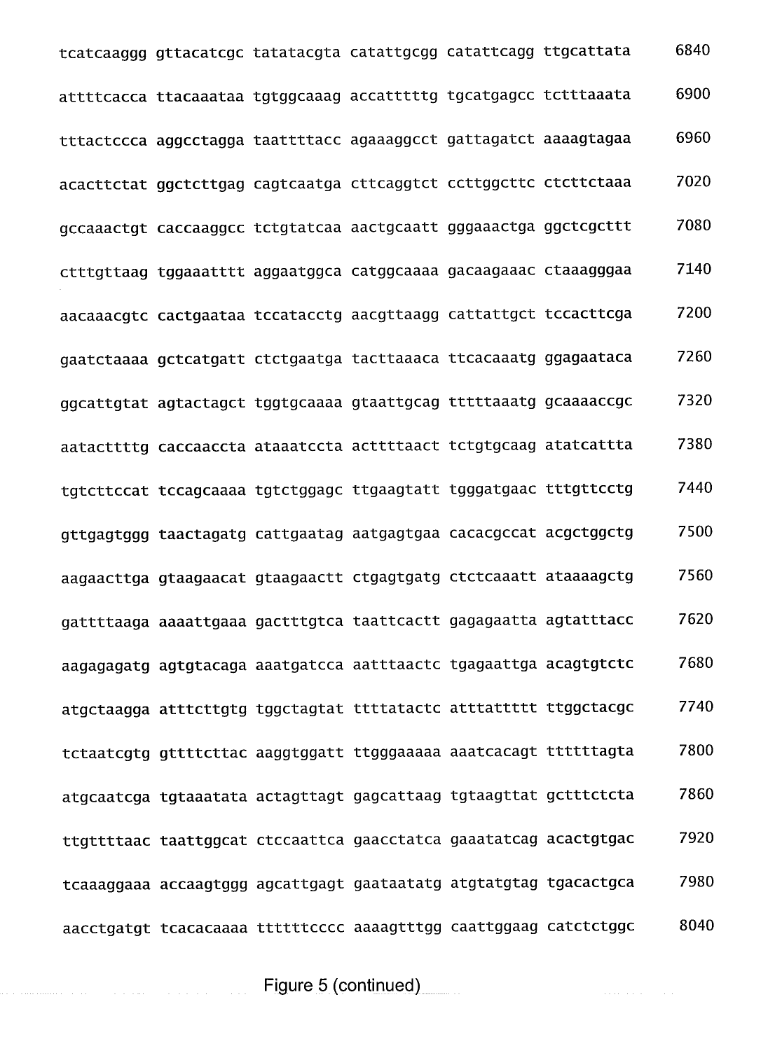

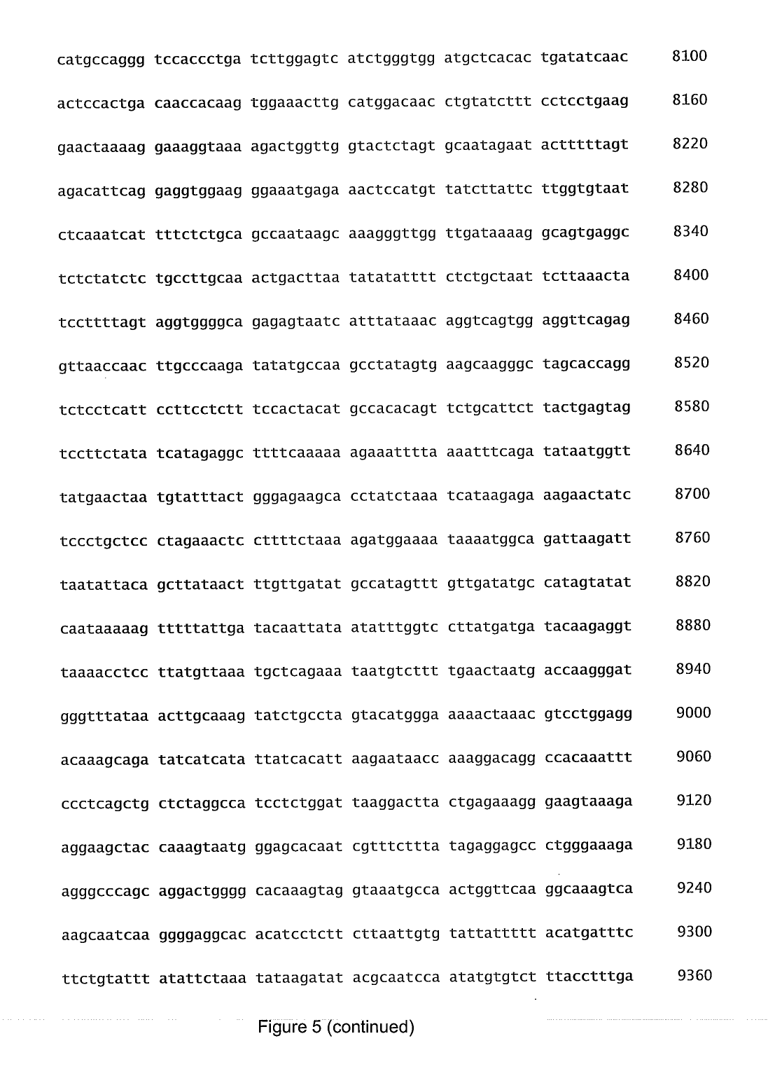

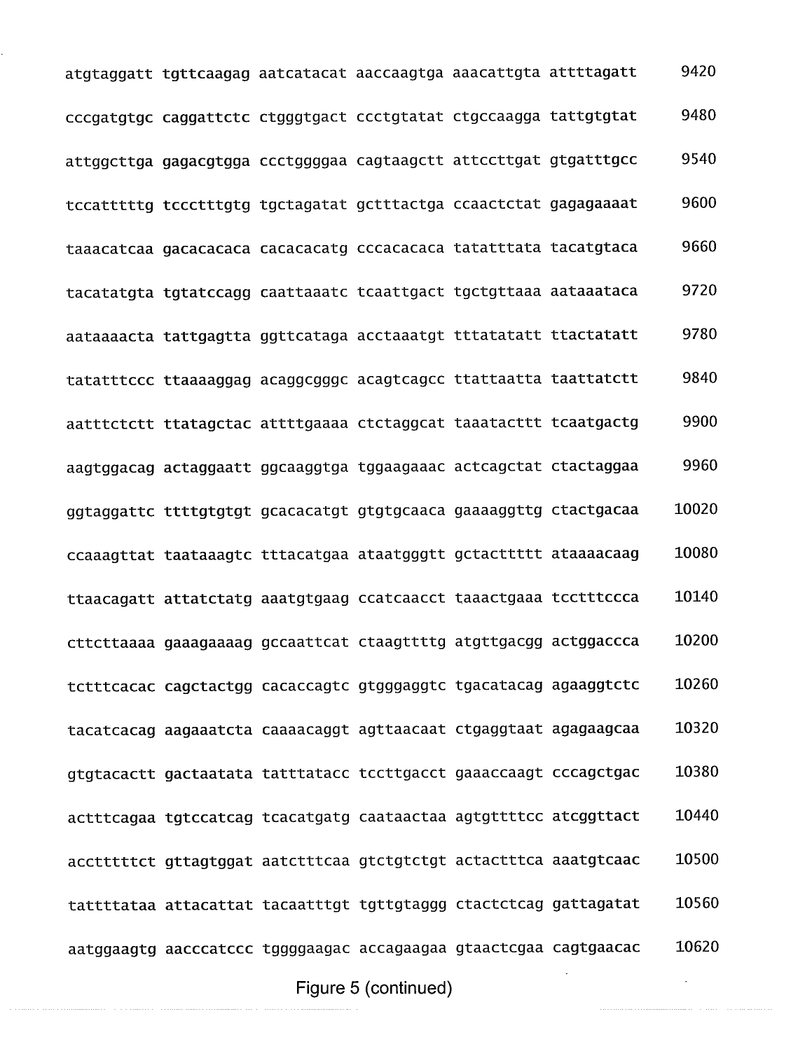

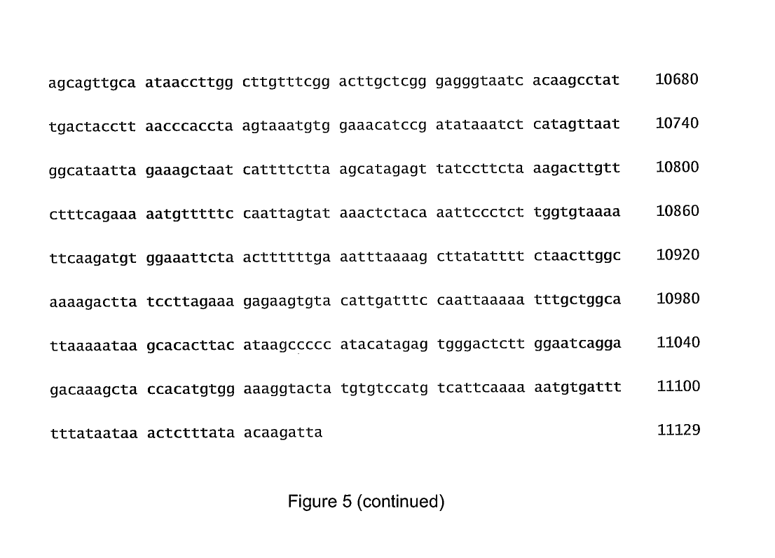

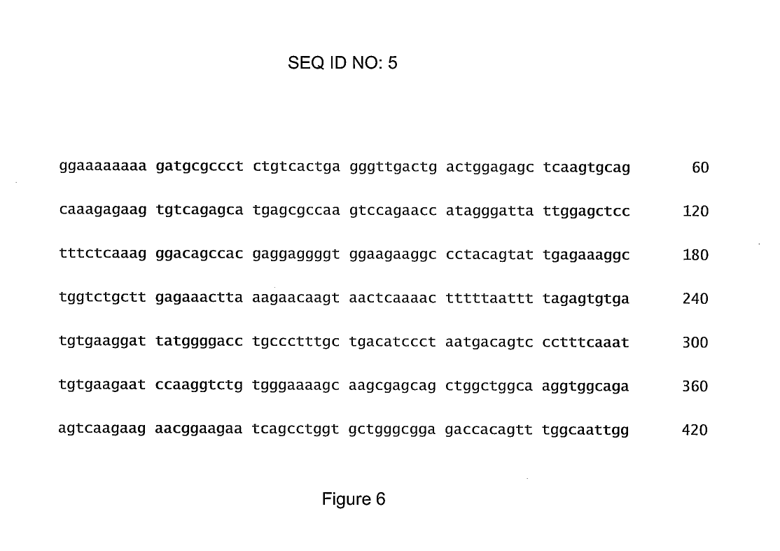

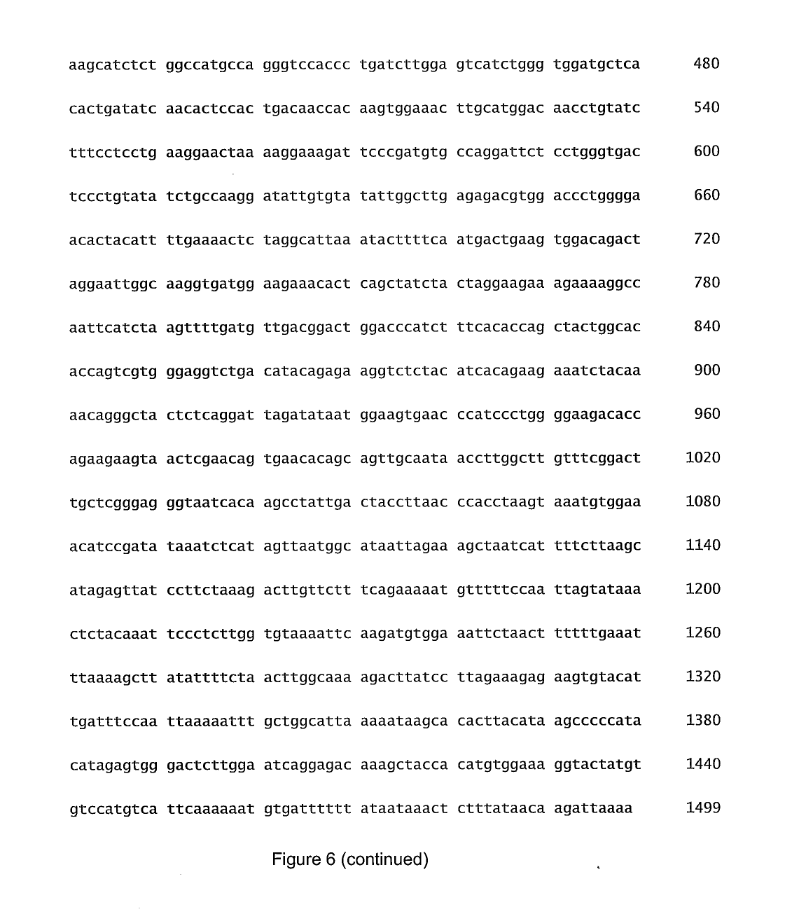

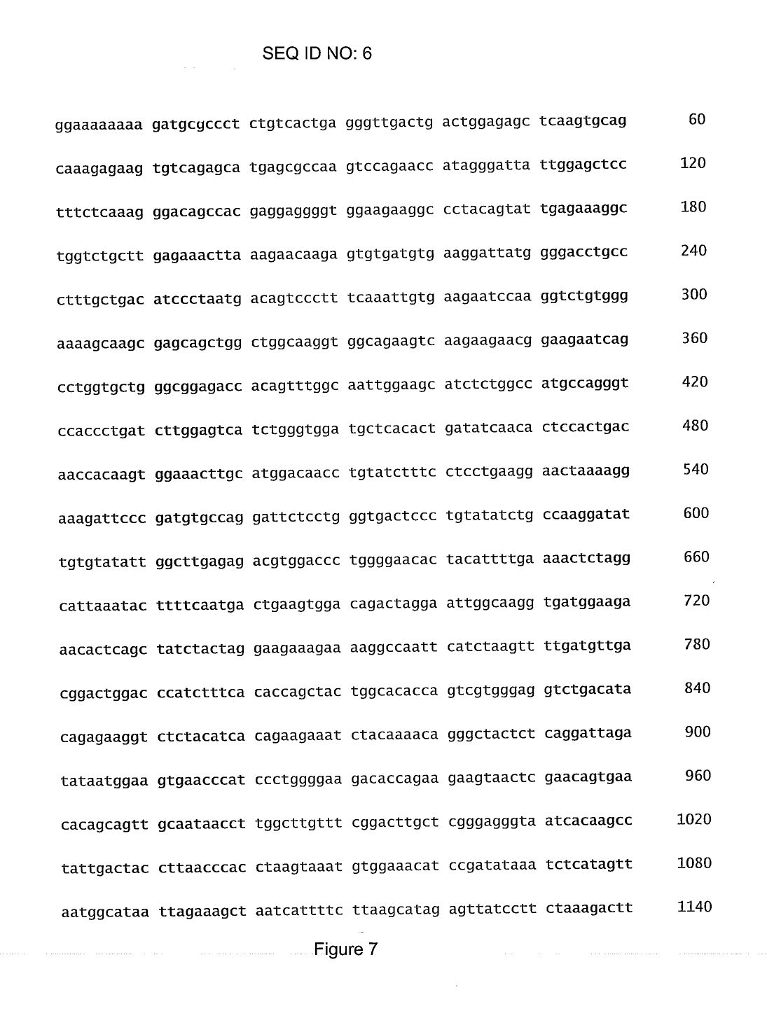

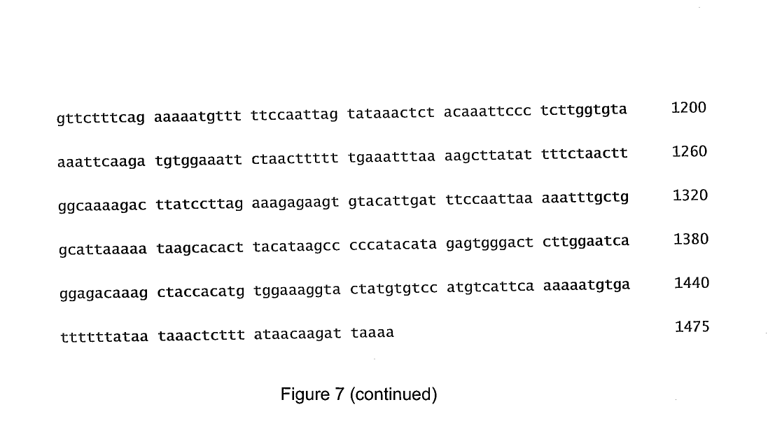

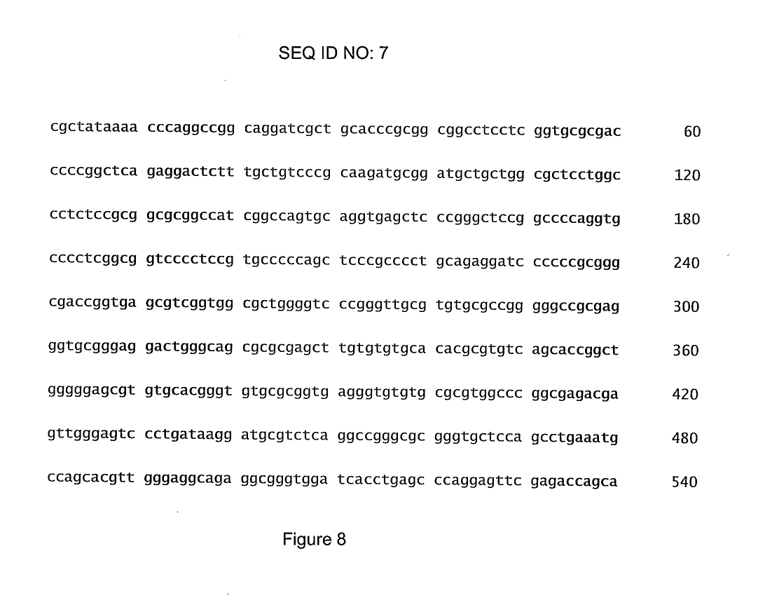

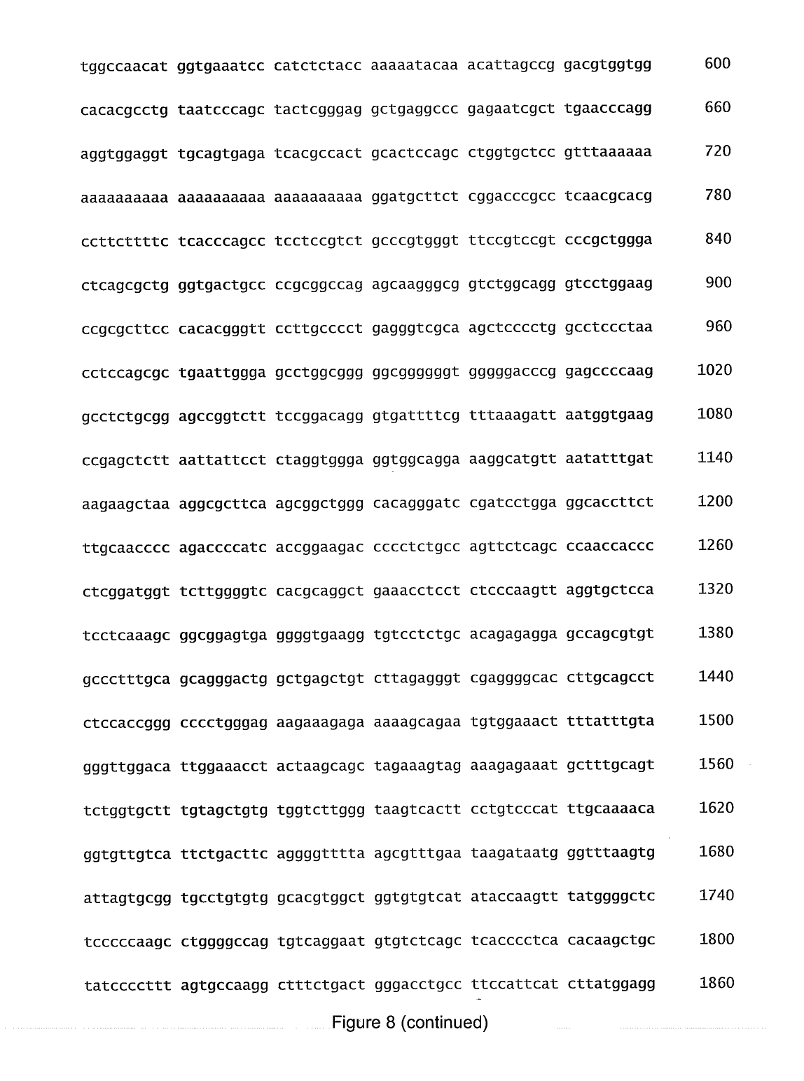

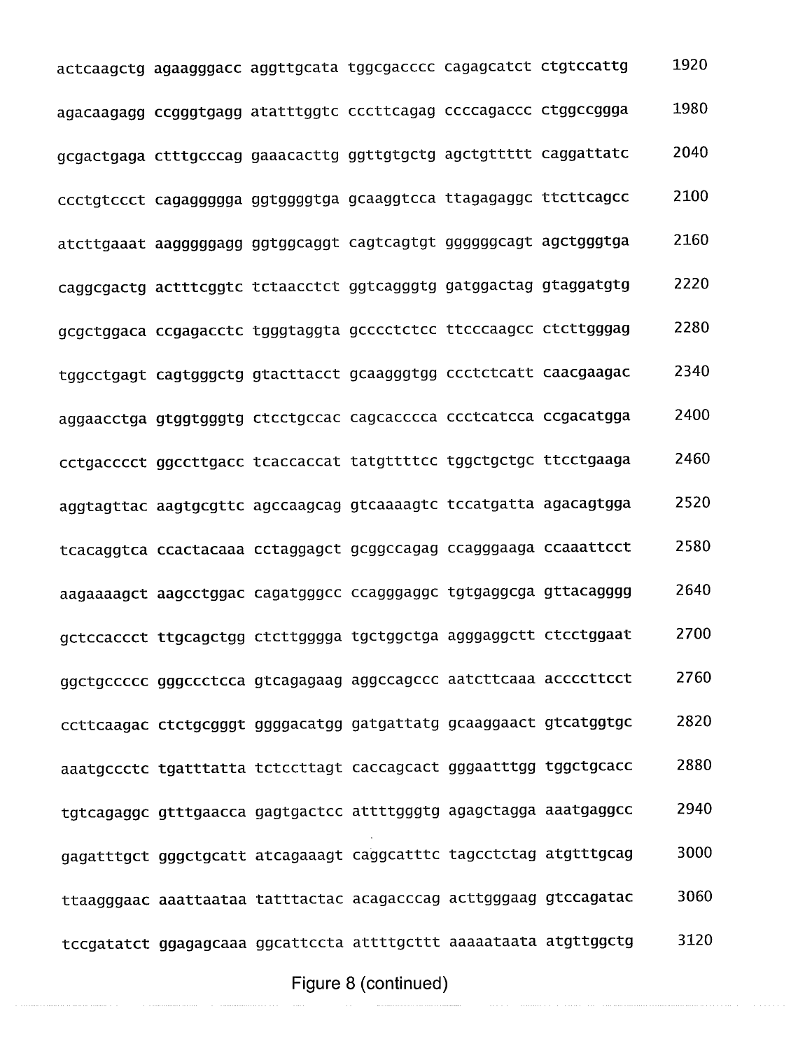

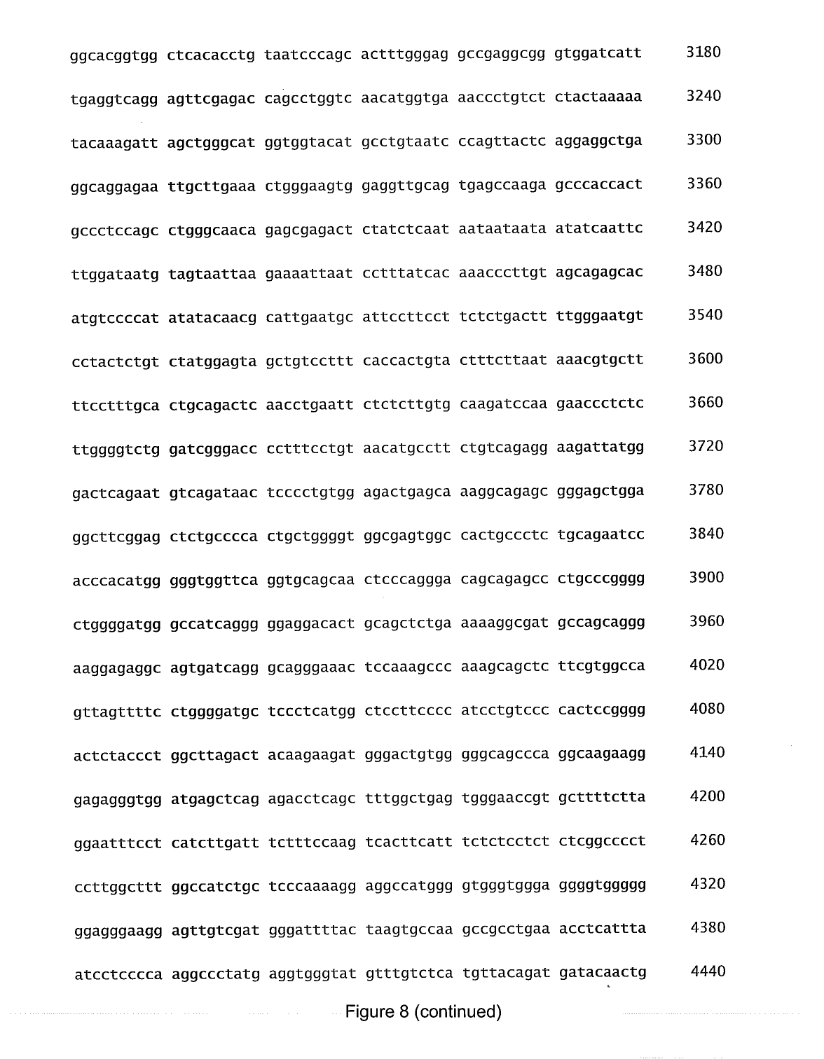

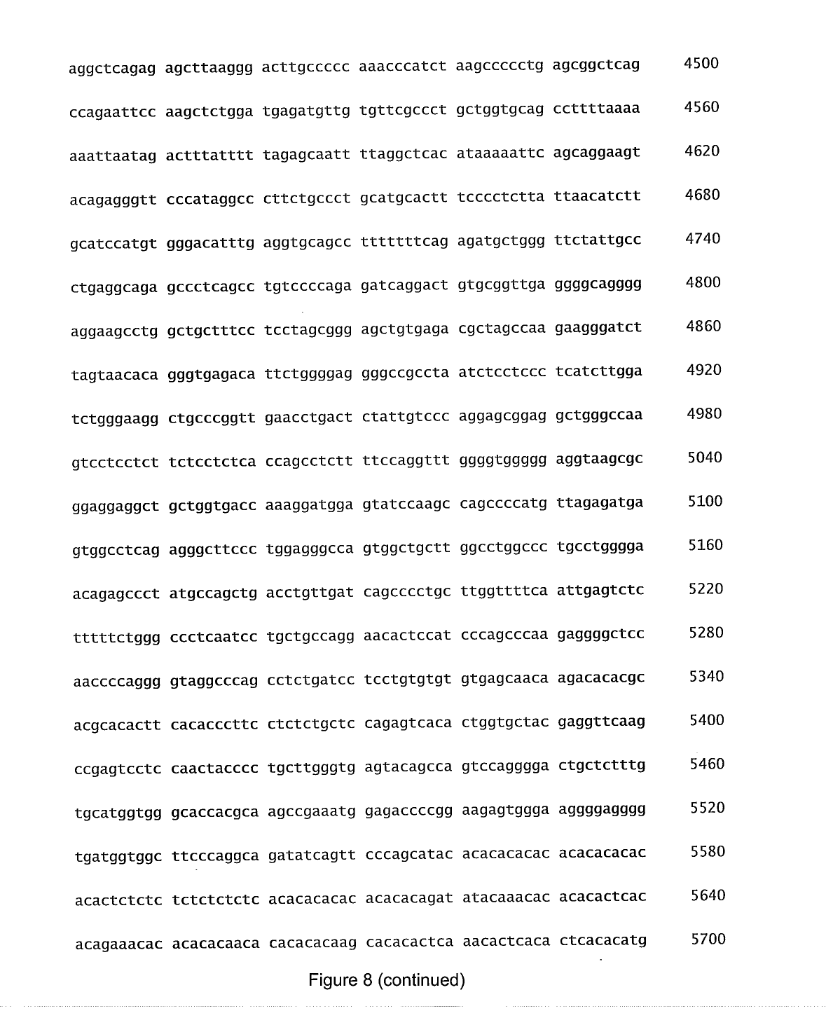

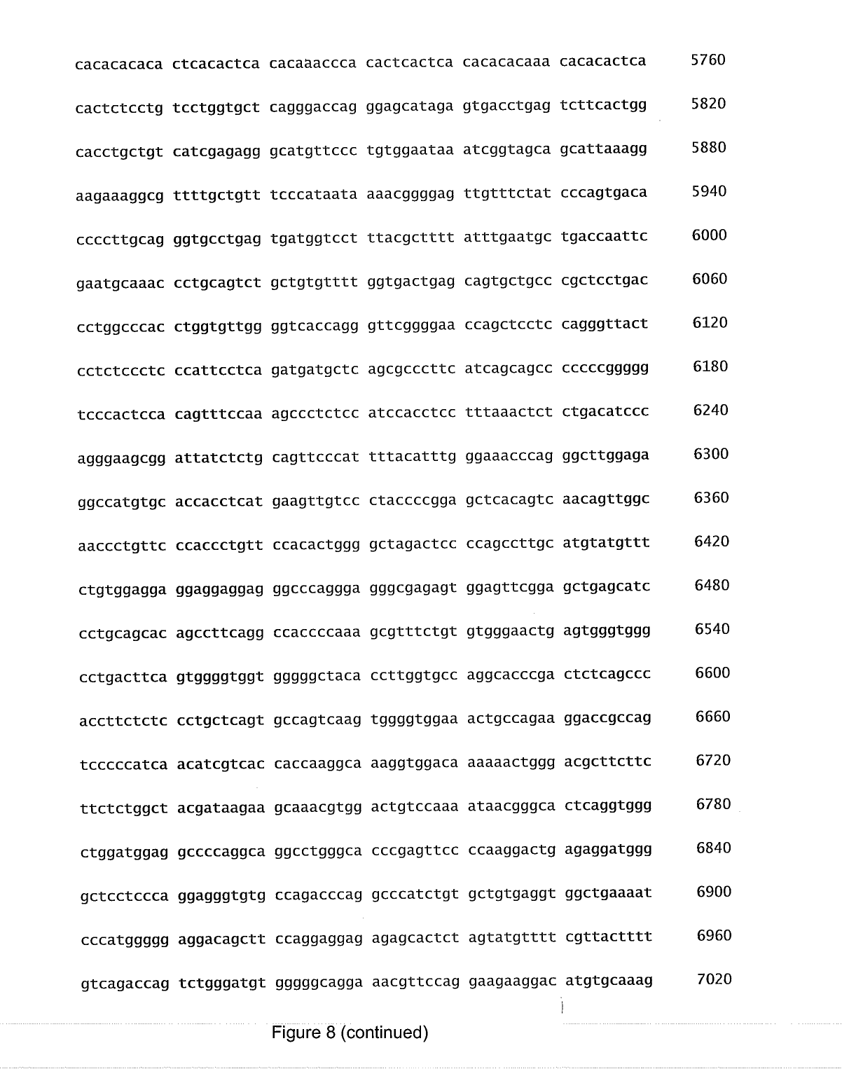

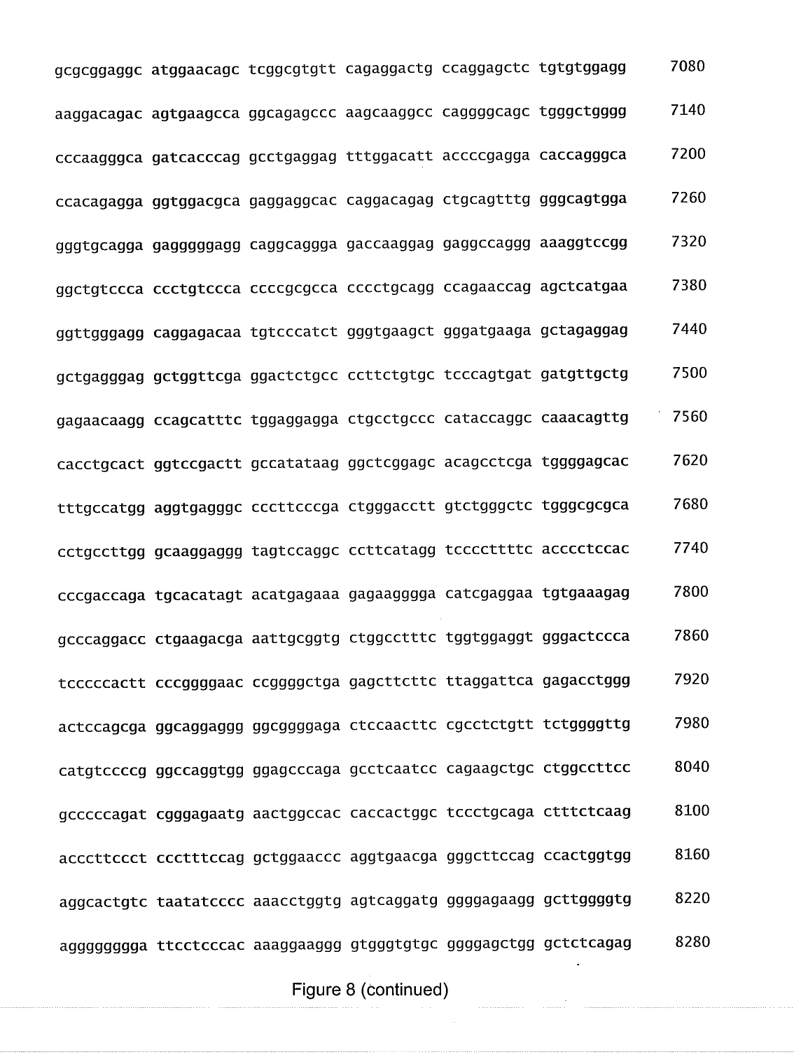

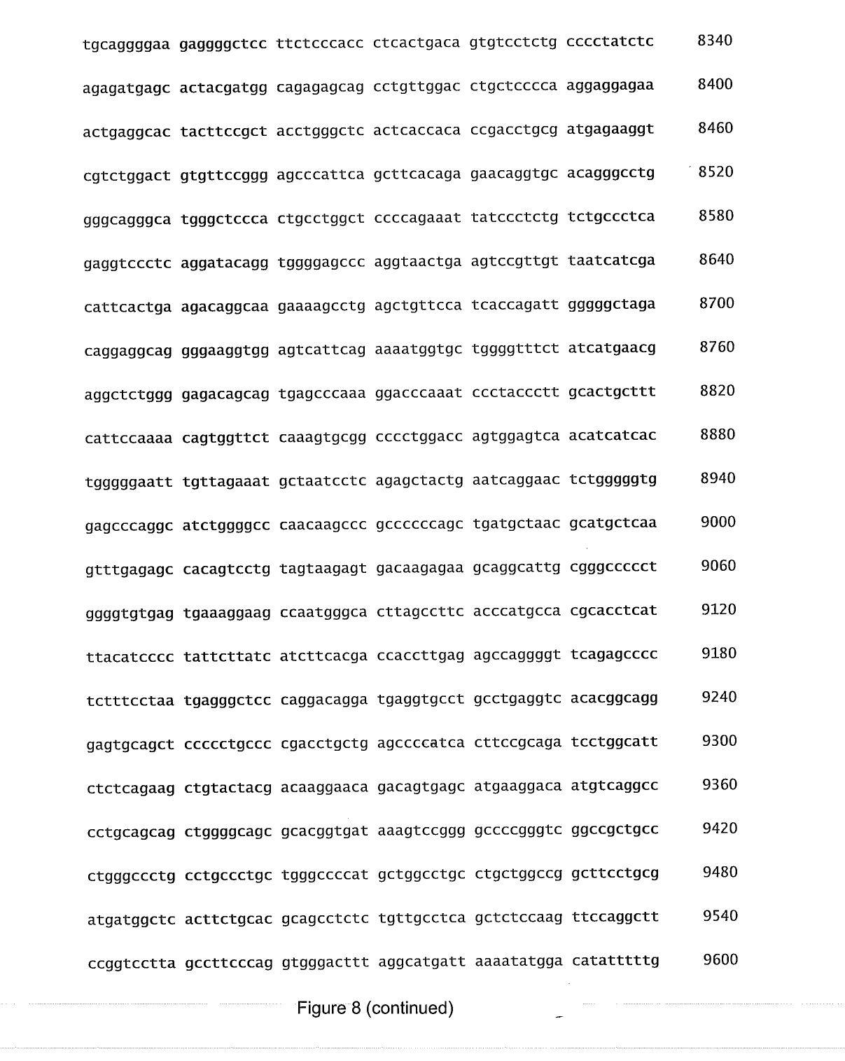

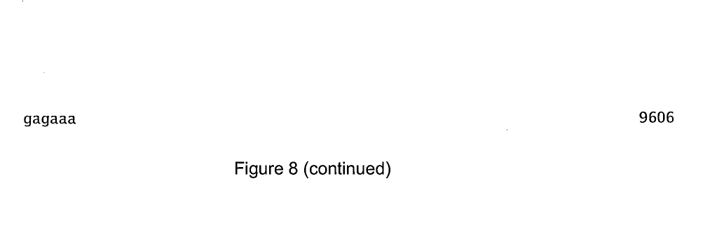

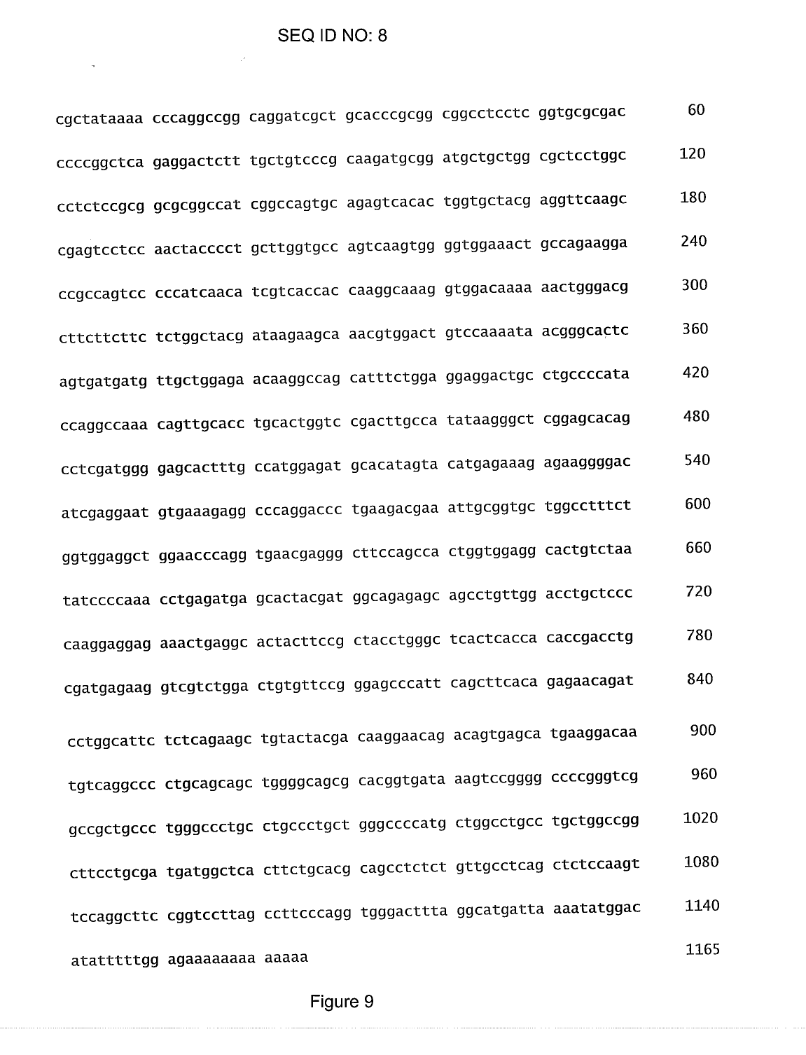

Peripheral whole blood samples were collected from MRI diagnosed IS (ischemic stroke) patients (here, human beings) greater than 18 years of age within 24 (twenty-four) hours from last known normal (i.e. pre-stroke status) and 24 to 48 hours later. Total RNA was stabilized in Paxgene RNA tubes extracted from whole blood, amplified, and hybridized to Illumina HumanRef-8v2 bead chips. Gene expression was compared in a univariate manner between stroke patients at both time points using t-test in GeneSpring. Inflation of type one error was corrected by Bonferrone. Linear regression was used to model the change in gene expression as a function of time controlling for age. Validation of microarray findings was confirmed with RT-PCR in a separate stroke patient cohort. FIG. 1 sets forth a table that shows patient demographic information. FIG. 1(b) is a graph of the expression of LY96 over time which shows that an increased time from stroke onset is associated with decrease expression of LY96. FIG. 1(c) is a graph of LY96 Ct gene expression over time that shows RT-PCR validation of LY96 wherein the LY96 raw Ct values show a decreasing trend over time with a small sample size. FIG. 1(d) is a graph of LY96 dCt gene expression over time that shows RT-PCR validation of LY96 when normalizing LY96 to B-Actin the decreasing trend is no longer seen.

It will be understood by those persons skilled in the art that the early administration of tPA after stroke onset has been associated with improved functional recovery of the patient, increasing the percentage of patients who receive tPA will significantly improve the current quality of acute care and increase the likelihood of positive outcomes. The data of the present invention provides evidence that the expression of LY96 in the peripheral blood serves as a surrogate for determining stroke time of onset. The present inventions method based upon this biomarker profile and other clinical covariates is useful when time of onset of stroke is unknown to provide clinicians with additional certainty to administer tPA. The method of the present invention may be used in conjunction with a point-of-care blood test for the diagnosis of ischemic stroke that shall increase the utilization of tPA or increase the time window of treatment in hospital based clinics and in the field.

A retrospective case-control study utilizing prospectively collected data from two different study sources was undertaken. Recruitment of stroke patients having the following inclusion criteria: age >18 years; MRI diagnosed definite Acute Ischemic Cerebrovascular Syndrome (AICS); and blood drawn within 24 hours from symptom onset. Patients with probable/possible AICS and hemorrhage were excluded from this study. Time of onset was determined as the time the patient was last known to be free of the acute stroke symptoms. rtPA was given to patients with disabling symptoms within 3 hours from onset. Pre-morbid deficits were determined by the Modified Rankin Scale (MRS) for status prior to stroke and severity of injury was determined by the National Institutes of Health Stroke Scale (NIHSS) at the time of blood draw after stroke. Control subjects were recruited as a consecutive convenience sample under a separate NIA/NIH protocol if they were neurologically normal per neurologist assessment at the time of enrollment. Peripheral whole blood was collected into Paxgene blood RNA tubes (PreAnalytiX, Qiagen) after consent. Demographic data was collected from the patient or significant other by trained neurologists.

### Standard Protocol Approvals, Registrations, and Consents

This study received approval for human subject's research from the IRBs of the NINDS and NIA at NIH and Suburban Hospital, Bethesda Maryland. Written informed consent was obtained from all subjects or their authorized representations prior to performing any study procedures.

### RNA Extraction and Amplification

Paxgene RNA tubes were inverted 8-10 times and placed in a −80° C. freezer until RNA extraction. Tubes were thawed on a rotating bed at room temperature for 24 hours prior to RNA isolation. RNA was extracted per Paxgene Blood RNA extraction Kit (PreAnalytiX, Qiagen). Globin reduction was not conducted on any sample in this study since it has been shown to have little impact on probe detection when using the Illumina platform (Applied Biosystems).

Biotinylated, amplified RNA was generated from the Illumina TotalPrep RNA amplification kit (Applied Biosystems). RNA quantity was determined by the Nanodrop and RNA quality was determined by A260/A280 ratio and the presence of two distinct ribosomal bands on gel electrophoresis.

### Array Hybridization

Samples were randomly hybridized to Illumina HumanRef-8 v2 expression bead chips, capable of analyzing >22,000 genes and alternative splice variants. Beadarrays were scanned by the Illumina BeadStation 500X and raw intensity values were saved in IIlumina's Bead Studio program manager. Sample labeling, hybridization, and scanning were conducted using standard Illumina protocols.

### Statistical Analysis

Baseline demographic statistics were conducted in SPSS (version 15, SPSS, Inc., Chicago, Ill.). Comparisons were made using chi-square analysis for: gender, race, comorbidities (hypertension, diabetes and hyperlipidemia), and medication history. Student's t-test was used to analyze the significance of age among the groups. The level of significance was established at 0.05 for two-sided hypothesis testing.

### Probe Level Analysis

Probe expression was filtered in GeneSpring GX v10 (Agilent technologies) resulting in a 24,424 final probe set. Robust multi-array analysis (RMA) normalization collated the probe data in the following order: 1) Background correction -perfect match probe information; 2) Quantile normalization-probe level normalization; and 3) Summarization-expression measure summary in log base 2 scale with median to fit a linear model. Unsupervised clustering was performed to determine phylogenetic distances to detect outliers.

### Gene Expression Level Analysis

Gene expression analysis was conducted in Illumina BeadStudio Gene Expression (GX) Module (version 1, Illumina, Applied Biosytems, San Diego Calif.) and verified in GeneSpring GX v10 (Agilent technologies). Genes with at least a 2 fold difference in expression were compared in a univariate manner between stroke patients and control subjects through the use of Illumina's custom model (modified t-test) in BeadStudio and t-test comparisons in GeneSpring. The influence of multiple testing was evaluated using the Bonferroni Family wise error (FWER).

### Logistic Regression for Identification of Off-Target Effects

Given the significant difference of age by group, a post-hoc logistic regression was performed. The normalized intensities for each gene were entered separately with age and then hypertension and dyslipidemia as the covariates of interest. A Bonferroni corrected p of <0.005 (0.05/9) was significant. A linear regression was used to model the change in gene expression as a linear function of time when controlling for age.

### Polymerase Chain Reaction Validation

cDNA was generated per Invitrogen, SuperScript III first strand synthesis kit. QRT-PCR reactions were performed using Taqman gene expression probes (Applied Biosystems) for ARG1, CCR7, LY96, and MMP9 by the 7900HT QRT-PCR system. Beta-actin normalized the relative expression of chosen genes. Fold change differences were calculated by the delta delta C_{T} method. Validation was confirmed if t-test revealed significance (p≧0.05) and QRT-PCR results correlated with microarray signal intensity (Pearson r≧0.5 and p≧0.05).

### Sample Size Estimation

Sample size estimation was conducted using PASS: Power analysis and sample size system and JMP. Twenty-two patients and 22 control subjects achieves 90.68% power for each gene to detect a difference in expression with at least a 1.5 fold change and a standard deviation of 1.5 with a false discovery rate of 0.05 using a two-sided one-sample t-test.

### Results

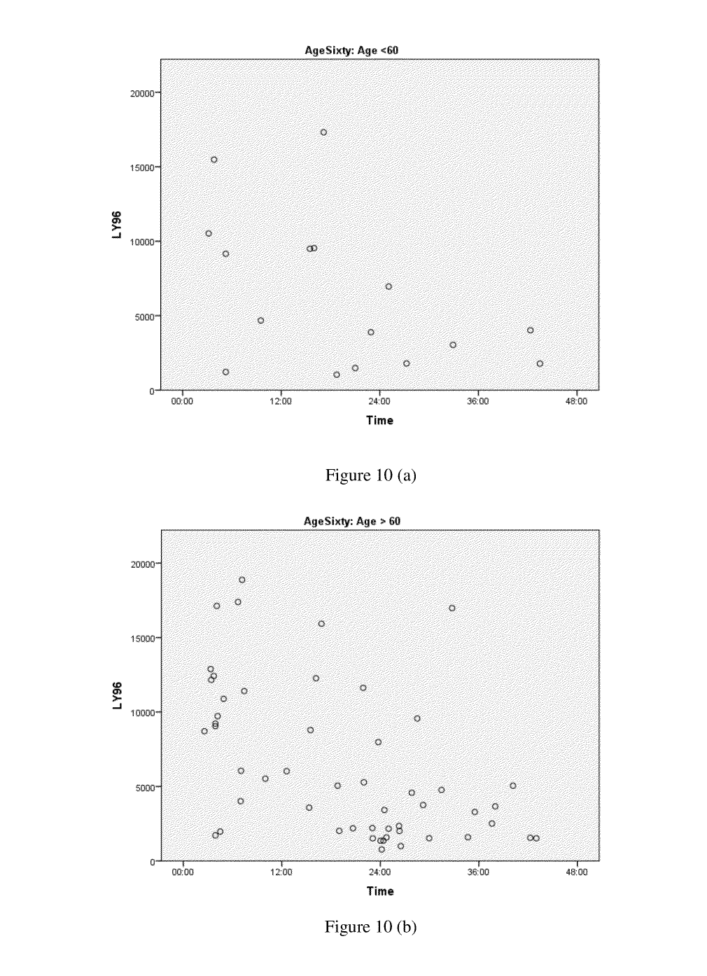

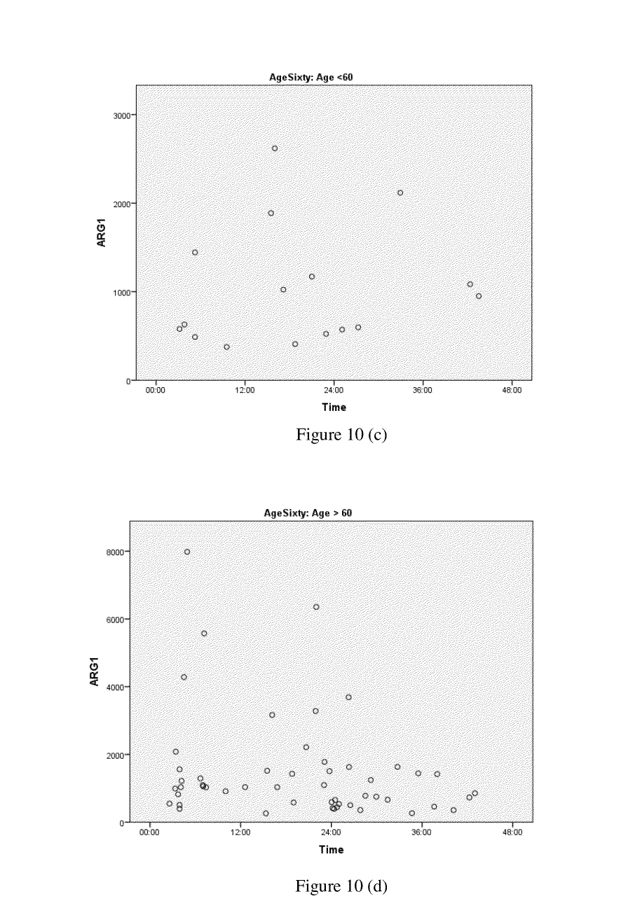

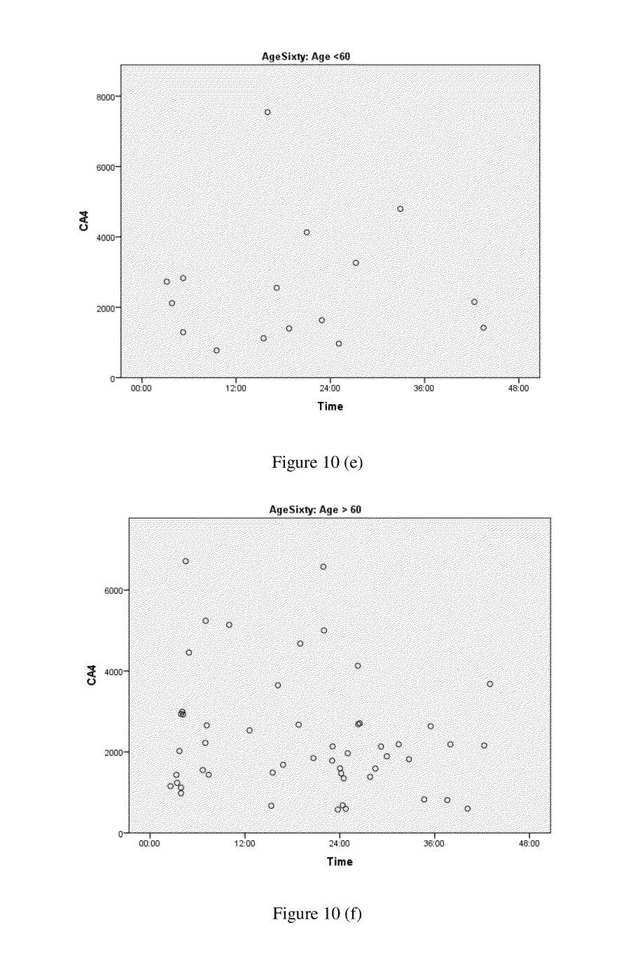

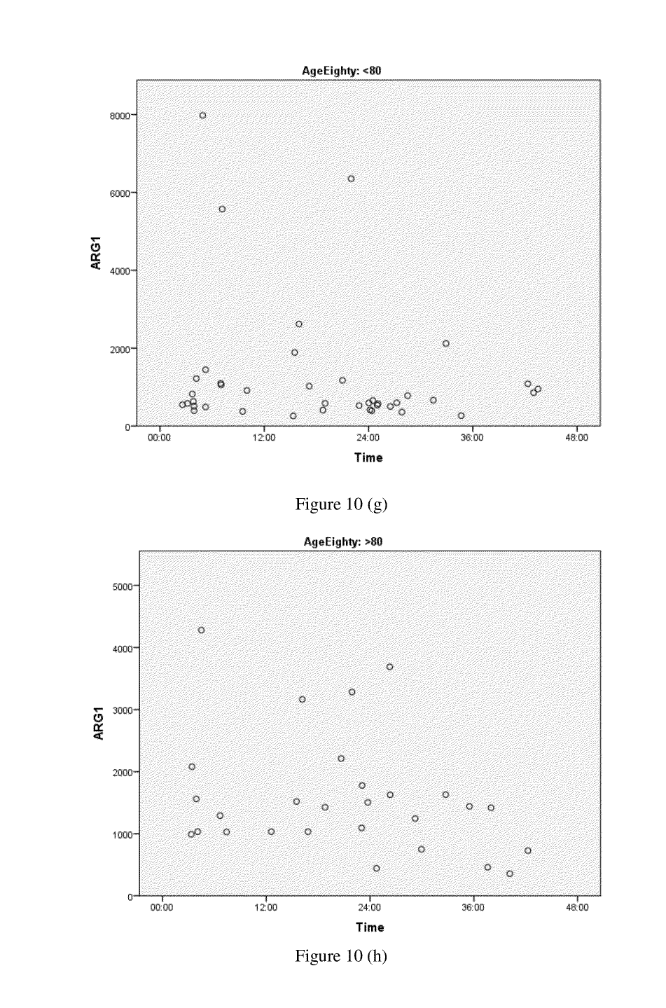

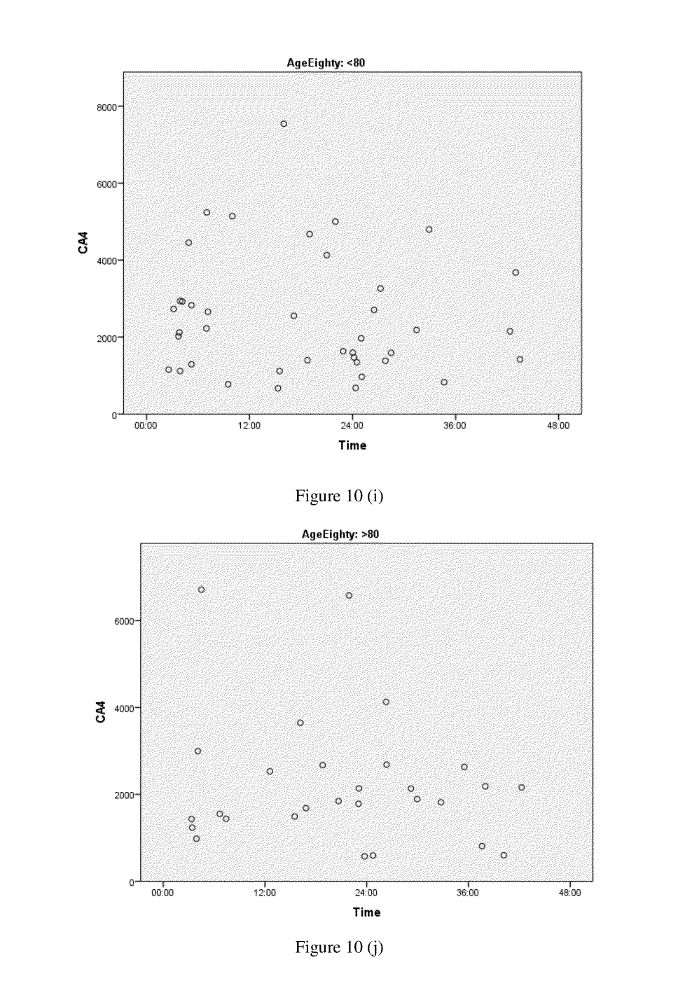

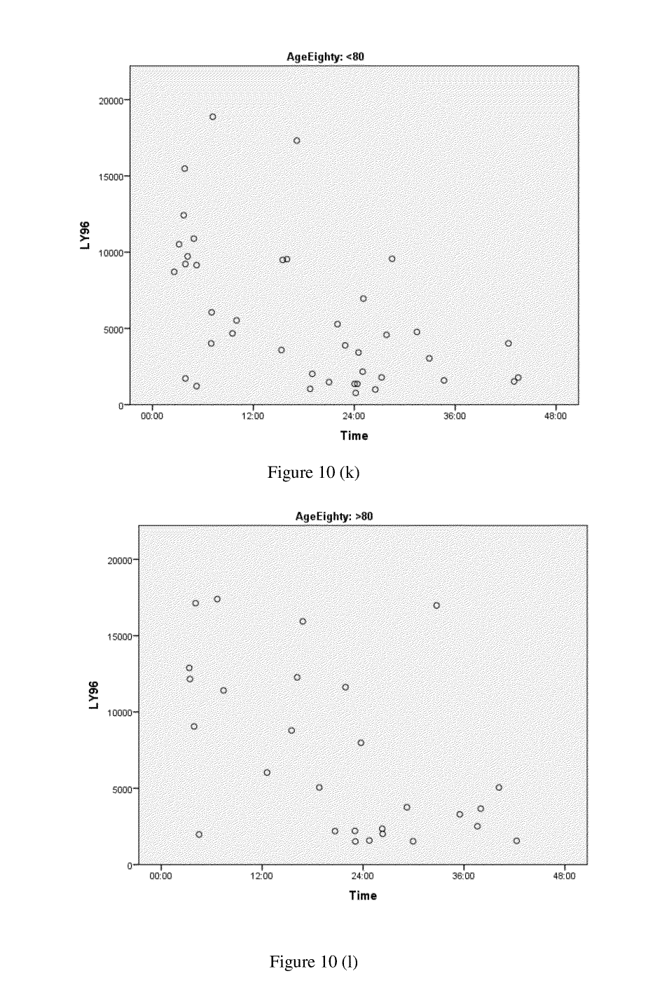

The mean age of the sample was 71.9±(14.6 sd) years. Mean time from symptom onset to acute blood draw was 9:29±(6:2 sd) hours (range 2:35-23:02); to follow up blood draw was 29:24±(7.1 sd) hours (range 18:45-43:30); and time between acute and follow up blood draw was 19:55±(3.3 sd) hours (range 13:30-27:32). CA4 and ARG1 expression significantly decreased >1.5 fold (FIG. 10), and LY96 expression by >2-fold between baseline and follow up (FIG. 1b). This decrease in expression was associated with an increase from time of stroke onset and remained significant for only LY96 expression when controlling for age. ARG1 and CA4 expression were significantly lower in older patients.

Whereas particular embodiments of this invention have been described above for purposes of illustration, it will be evident to those persons skilled in the art that numerous variations of the details of the present invention may be made without departing from the invention as defined in the figures and the appended claims.

What is claimed is:

1. A method of determining the time of stroke symptom onset comprising:
obtaining a biological sample from an individual;
contacting said biological sample with a detection composition comprising at least one of a lymphocyte antigen 96 (LY96), an arginase 1 (ARG1), and a carbonic anhydrase 4 (CA4) expression mediators, or a combination of said expression mediators, wherein at least one of said expression mediators is associated with an acute phase response of ischemic stroke, for forming a detectable response; and
correlating said detectable response with a time of onset of one or more stroke symptoms.

2. A method of determining the time of stroke symptom onset comprising:
obtaining a biological sample from an individual;
contacting said biological sample with a panel of detectable polynucleotides or functional polynucleotide fragments which correspond to an expression mediator of at least one of a LY96, an ARG1, and a CA4, or a combination of said expression mediators, wherein at least one of said expression mediators is associated with an acute phase response of ischemic stroke;
forming a detectable response; and
correlating said detectable response with a time of onset of one or more stroke symptoms.

3. A method of determining the time of stroke symptom onset comprising:
obtaining a biological sample from an individual;
contacting said biological sample with a panel of detectable oligonucleotides which correspond to at least one of a LY96, ARG1, and CA4 expression mediators, or a combination of said expression mediators, wherein at least one of said expression mediators is associated with an acute phase response of ischemic stroke;
forming a detectable response; and
correlating said detectable response with a time of onset of one or more stroke symptoms.

4. A method of determining the time of stroke symptom onset comprising:
obtaining a biological sample from an individual;
contacting said biological sample with a panel of detectable antibodies for at least one of a LY96, ARG1, and CA4 expression mediators, or a combination of said expression mediators, wherein at least one of said expression mediators is associated with an acute phase response of ischemic stroke;
forming a detectable response; and
correlating said detectable response with a time of onset of one or more stroke symptoms.

5. A method of determining the time of stroke symptom onset comprising:
creating a sample by extracting target polynucleotide molecules from an individual afflicted with an ischemic stroke so that the RNA is preserved, deriving the mRNA from the mRNA of the individual, labeling the mRNA and hybridizing to a detection mechanism containing at least one of a LY96, an ARG1, and a CA4 expression mediators, or a combination of said expression mediators, wherein at least one of said expression mediators is associated with an acute phase response of ischemic stroke;
forming a detectable response; and
correlating said detectable response with a time of onset of one or more stroke symptoms.

6. A composition for the detection of biomarkers comprising:
a nucleic acid probe that is specific for at least one of a LY96, an ARG1, and a CA4 expression mediators, or combinations of said expression mediators.

7. A composition for the detection of biomarkers comprising:
at least one antibody that is specific for at least one of a LY96, an ARG1, and a CA4 expression mediators or a combination of said expression mediators.

8. A composition comprising:
a purified biomarker specific for at least one of a LY96, an ARG1, and a CA4 expression mediators, or a combination thereof, and the corresponding encoding nucleic acids thereof.

9. A method for determining the time of onset of ischemic stroke symptoms or other neurological disease comprising:
creating a sample by extracting target polynucleotide molecules from an individual afflicted with an ischemic stroke so that the RNA is preserved, deriving the nucleic acids from the mRNA of the individual, labeling the nucleic acids and hybridizing the labeled nucleic acids to a detection mechanism containing probes that are a portion of at least one of SEQ ID NO:1, SEQ ID NO:2, SEQ ID NO:3, SEQ ID NO:4, SEQ ID NO:5, SEQ ID NO:6, SEQ ID NO:7, and SEQ ID NO:8;
determining a chemoresponse based on gene expression profiles between the sample and said detection mechanism; and
correlating said chemoresponse with a time of onset of one or more stroke symptoms or one or more symptoms of neurological disease.

10. The method of claim 9 including wherein said neurological disease is selected from the group consisting essentially of at least one of multiple sclerosis, Alzheimer's disease, migraine, epilepsy, and traumatic brain injury.

11. A method for determining the time of onset of ischemic stroke symptoms or other neurological disease comprising:
creating a sample by extracting target polynucleotide molecules from an individual afflicted with an ischemic stroke so that the RNA is preserved, deriving the nucleic acids from the mRNA of the individual, labeling the nucleic acids and hybridizing the labeled nucleic acids to a detection mechanism containing probes that are a portion of at least one of SEQ ID NO:2, SEQ ID NO:3, SEQ ID NO:5, SEQ ID NO:6 and SEQ ID NO:8;
determining a chemoresponse based on gene expression profiles between the sample and said detection mechanism; and
correlating said chemoresponse with a time of onset of one or more stroke symptoms or one or more symptoms of neurological disease.

12. The method of claim 11 including wherein said neurological disease is selected from the group consisting essentially of at least one of multiple sclerosis, Alzheimer's disease, migraine, epilepsy, and traumatic brain injury.

13. A method of determining the time of stroke symptom onset comprising:
obtaining a biological sample from an individual;
contacting said biological sample with a biomarker comprising at least one selected from the group consisting of a lymphocyte antigen 96 (LY96), an arginase 1 (ARG1), and a carbonic anhydrase 4 (CA4), or a combination of said biomarkers, wherein at least one of said biomarkers is associated with an acute phase response of ischemic stroke, for forming a detectable response; and
correlating said detectable response with a time of onset of one or more stroke symptoms.

14. A kit comprising a detecting mechanism for detecting at least one biomarker that is diagnostic of an ischemic stroke, said biomarker selected from the group consisting of a lymphocyte antigen 96 (LY96), an arginase 1 (ARG1), and a carbonic anhydrase 4 (CA4), or a combination of said biomarkers.

15. The kit of claim 14 wherein the biomarker is one selected from the group consisting of a nucleic acid, and a polypeptide.

16. The kit of claim 14 wherein the detection mechanism is a filament-based diagnostic system capable of detecting either a nucleic acid molecule biomarker or a polypeptide biomarker.

17. A filament-based diagnostic system comprising either (i) a panel of detectable polypeptides or functional polypeptide fragments thereof each corresponding to, (ii) a panel of detectable oligonucleotides each corresponding to, or (iii) a panel of detectable antibodies, each capable of specifically binding, an ischemic stroke biomarker selected from the group consisting of a lymphocyte antigen 96 (LY96), an arginase 1 (ARG1), and a carbonic anhydrase 4 (CA4), or a combination of said biomarkers.


SEQUENCE LISTING (8 sequences)

SEQ ID NO: 1 - DNA, 37751 bp, Homo sapiens
  ref <308>: DATABASE ACCESSION NUMBER: NCBI / NC_000008
  ref <309>: DATABASE ENTRY DATE: 2002-08-29
  ref <313>: RELEVANT RESIDUES IN SEQ ID NO: (74903564)..(74941314)
agaaatcatgtgactgatgactaagttaaatcttttctgcttactgaaaaggaagagtctgatgattagttactgatcctctttgcatttgtaaagctttggagatattgaatcatgttaccatttctgtttttttccaccctgttttcttccatatttactgaagctcagaagcagtattgggtctgcaactcatccgatgcaagtatttcatacacctactgtggtaagtaaaaccgcaaaacaaataattgtagcatcaactattttgagggtaagttttcacgagaaccgtacactgttgtggctggaacacacgaaacatcagtgtgttccagctgctgtggcggacgctgccagcaggaaaagcaatagctggcagctgccccatgagaatgttctcgaccactcacagagcttgtgagtctctaaactgtgttgtgcttgacctccaagtgcttctgttccctgcgtcatctttctctaccctgagcccagtttttggttctgcccatttttaataaaaccatctgcttctgagctttcacctcagccttgacacttactagttcttcttcttgagatggtagagcagtcccagctgcttcctggagttgatttctgctctaagctgtgactcttgagtgctcttggaatcatccccctcactggatgaaagcctggttctgtcactagctgtgtgaccttaagcaaagtactccagcctttttagcactgaagtgaaagtgattttatagtctacttgataagggtgttgtgaggtgaagttttttagcatagggtcacatagagacaataatactcttaatttttttttggagtcagccataccctaatactattcactggctgtgtcactgtcccctgccccagctgctcattagcaagtgtcctgggatctgaaggcacagtgtcaggcctgacagctgttttggttcggtactttccaccgtgctgcttaccatgggttggctgcccttttccacccactagcaatagctgggaggtttaaagtggtgcttcccaaaataccagttctttgagattcttttgggaaaataggatctatgacctaatcatctgggagagatttacagatgtactttgggttctgagaagttctgcaataaaggcacctgtttaaccacattttcaatttcatttgttgtttgtttgtttgtgttttagtctggagtgtatgttaactctcttgagaaacatgctctgccaaatgctaatataaagtgactttcgggaaattgggataattgccacatgtgaggaggagacttaggagtgtggccagtcacacaccatggcccttctcctggtgggttgcactgaattccctccaaacgtatgtctaaaatctctccatgctgcaagatccttttgcattcccctcttccagcaaggctcctactctacttggcccccacttccctctcctcctttggctcttttgtaccatgtgctaattatgccactgtgtgtgtgtgtgtgtgtgtgtgtgtgtgtgtgtgtgtgacagagttttgctctgttgccaggctggagtgcagtggcgcgatttcggctcactgcaacctctgcctcccaagttcaaacgattcccatgcctcagcctcctgagtagctgggactacaggtgtgcgccaccacacccggctaattttttttttttttgtattttagtagagacagggtttcatcatgttgaccaggatggtctcaatgtcctgatcttgtgatctgcccaccccggcctcccaaagtgctgggattacaggcgtgtgccaccttgcccggtttatgccactctttatatggtttttatggatgtaacattttgtattattatttagctttcaatcttttcttaacaataactcaatttgtctttttaaaacatttcacattttatcttattttatcatttatttttgagacagggttttgctctgttgtgcaggctggagtgagtggcatgatctccactcactgcaacctccacctccctggctcaagccatcctcccagctcagccttccaagtagctgggactacaggcacatgccaccacacttgtctaattattaattaaataattttttttgagtttcactcttgttgcccaggctggagtgcaatggcatgatcttggctcactgcaacgaccgcctcctgggttcaagtgattctcctgcctcagcctcccgagtagctgggattataggtgcccaccaccacacccagctaatttttgtatttttagtagagacagggttttgccatgttgaccaggctggcctccaactcctgacctcaggtgatccgcccaccttggcctcccaaagtgttgggattacaggcgtgagccaccgcagctggcctaattattaatttttttgtagagatggagtcttgctatattgcccaggctggtctggaactcctgggctcaagtgattctcccaccttggcctcccaaagtgctgggattacagatatgagccactgcacctggcctaaaatttcacattttaatcatttttgagtgtttagctcaattgtattaagtatatgcacattgctgtgaaaccatcaccgttggtgtatcttttgatttgactttctagtttttttttttactttgagacagtctcattctgtcatctaggctggagtgcagtagtacgatctcagctcactgcaacctctgcctcctgggttcaagcgattcttttgcctcagcctcctaaatagctgagattacagatatgcaccaccatgcctggctaatttgatttgactttatgaaggaggatcttccttccttctctctgagaaaccctgtggtcctttcctgctgccccactgacaaagggactgtgggcaagccactggaatacacatctacagcaagatgttgcttgttgccatccccactgccatatgactgccttcccggcctgagagaatccatcctgcccagccaaaaatgcatcacctgtgggaagcactgattcctctaggtaccattgtgagggtgttggtgacaacaacctctgattcactgtgatgatttccatgaaaaagaagagatagatttccatttggtcaagctgctgggcatctaagtgtatgtttttttaaacacagagtcttgctctgtcagccaggctggagtgcaacggtggaatcatggctcactgcagcctcaaactctgaggttcaagtgattttcccgcttcagcctcccaagtagctgggactacaggcgtgcaccaccatgtctggctaatttttaagttttttgtagtgatggggttttgccatgttgtccatgctggttttgaactcctggtctcaagcaatcttcctgcctcagcttcccaaagtgctgggattacaggcctgagccactgattccgggctacagtgtcttaattagttcaggtgccataacaaaataccacagactggatggctcaaacaacaaaaacctattttttcacagttctggaggctagaagtccaagatcaaggtgtctgcataattgggttctcctgaggcctctctccttggcttactgctgtgttttgaattttgtgtccttgcaaactcatgttgaaatttaattgccattgtgatgatattaagaggtgggacttttaagaggtgattaagccatgaaggctcttctctcatgaataaattaatgtcattattgagggagtgagtttctcataaaaagacaagtttagcccccttatgtctcctgcttttgccccctcttgcccttctgctcttccaccatgggatgagacagcaagaacgcccttgtcagataccagcaccttcacatggaacttaccagcctccggaactgtgagaaataaatttctgttctttattaagtacccagacaccaataatctgttatagcagcacaaagtggactaagacactcgcagctggctgctttctcactgtgtcctcacatggcctttcctctgtgtgtgcacatccctggcgtttctttgtgtgtccaaatttcctcttcttataaggacaccaacaagattggattagggcccatcctaacagcctcattttaacttaattacctctttaaaggcactgtctccaaatgcagtcatattctgaggtactggagccttaacatgtgaatggggacgagaggaatgaggacacatttctgtcggtccataacatacaaaaatggcaaaaattgactgatttattctcccagttttggaaaaggcaaaaaatgtgggtgtgtgagttggttcctaggatcttaggcacatgctttcctatgatcctgagtcattggtctcagttctaatctttattttttttttgctacagatggttagaaaatccaaggaattaatagattatttagtgaaaccaatattctctgagcatttagcatgcacttactctaggaactgaggagataagtgaattaacaacaaagacaaaagtccctgcccttatggattcagtaaaatattcagcatgtcagatggtggtaattgctgtgaaagaaaaataaggctgggggatgcggggtagagggtgcaaagatgcaaaaatggagaagctagggggttgcattttaaatcaggatggccagccagatgtgatggcactcaccagtagtatagcccagctactcaggaggctgaggcaggagaatagcttgagcccaggaggtcaaggctgcagtgagccatgtttgtgccactgcactccagtctgggcaacagagcaagactgaacctagctaatttttaaattttttgtagagacagtgtcttgctgtgctgcccaggccagtcccaacccctgggctcaagcagttctcctgccttggccttccaaagtgctggaattacatgcatgagccaccacgctaggcctaaaacctgtttcttttccttccttccttccttccttccttccttccttccttccttccttccttcattcctttctttctttctttctttctttctttctttctttctttctttctttctttctttctttctttctttttctgagacagagtcttgctgtgtcactgaggctggagtgcagcacgatctcagctcattgcaacctcccaggttcaagcgattttcgtgcctcggcctcccaagtagctgggactacagccatgcatcaccatgcctggctatttttttctgtttttagtagagacggggtttcaccatgttggtcaggctggtcttgaactcctgacctcaaatgatccacccaccttggcctcctaaagtgctggaattacaggcgtgagccactgcgcctggtcccggctaatttttgtattttttgggggatgggatttatttttttgagatggggtcttgctctgttgcccaggctggagtgcagtggtgcaatcttggctcacagcagtctctgcctcctgggttgaagtgattctcctgcctcagccttctgagtagcagggattacaggcctgcgccaccacacccggctaatttttgtgtttttagtagagacggggtttcaccatgttggtcaggctggtctcgaacttctgacctcagacaatccacccgcctgggcctcccaaagtgctgggattacaggcataagccaccatgcctggtctgtatttttttagtagagaccaggctgatcttgaactcctgattttaggcgatcagcctgccttggcctcccaacgtgtcgggattacaggcatgagccaccgtgtccggccttaaacctgtttctgtctaagtcttctccatctcatcatccacctaattccttaggccccaaaccagaagtatccttaagtatctctttccctcacctattacattattgtattagtcattcctgttttcatgatctccagattctattctatatccaagtgcttctcactgctctctaacactcatgccacatttgtctctccttggcctctacaaaaacctcttaaattgccctccctctgcttcagttcttgccaccatacaatctattcttgacacagaggccagagagatctttgcaaaatgaaaataagttagataatgtgacccctcctgctgaaaatcctccagtagctttcactacaattagagtgaaatctcaactttataccaagacctccaggactcctcaggatcttgaagcaatactaagactacctagaattacagtctaattagggggtccttcccacaagttttcctcccacatcagatgccatgtgcaagccctaggctgacacctgtggttctcactggctataaattggaggttcccctttcttgggtttgatcattgttagactaactctcagaacacaaggaaacactcatttatgtttactggtttattataaaggatattattaatacaaaggatacagacgaatagccagatgaaagagatatataaggtaaggtgcaggataaggggaccagagtgtccatgtcctctctgggtgcaccatcctcctagcatctccatgtgttcaccaacccagaagctctcctaaacctgtcgtccagaaatttttatggggtgttcatcatgtaggcataatcgaatgttaactctgtctgtagcccttctccccttcctgcaggatgggaggtggggctaaaagttccaagcttctcatcatggcttggtctttctcataactggcccttatccaggaacccaccaaaagttaccattagaaaaaaagatgctgctattgcccaggacagtctaaggaactagaagctctgggcaagaaattgggatcaaaaaccaaatgttaggaccaaaaattctcttaccactcccattgttctgtaaactacaagggttttaggatctctgtgccataaactggggcagagacaaaatatatgtttcttattatatcacagtatcacaaattctgtacacattttatagttttttttttctagttctgtaaaaaatgtcactggtgccaggcatggtgtctcatgcctataatcctagtgctttgagaggctgaagtgagaggatcacttgaggccaggagttcaagaccagcctaagcaacatagtgaggccccatctctacaaaaaaatgaagaaaaaaaaattcactgggtgtggtggcatgcacctgtagtactagcgactcaggagactgaggtgggaggatctcttgagcccaggagtttgaggttgccctgagctatgattgtgccactacactttatcctgggtgacagagtgaaatcctgtctcttctctctcaaaaaaaaggaaaaatgccattaggattttgatagagattgcttcaaatctggatctgtaggacactttgaatggtgtgaacattttagcaacattgtcttccaatctatgagcaagggatatctttctgtttctttttgtcctgttaatgtctttgatcaatttattttattgtttttactataaaagtttttaacttccttagtgaagtttattgtattcctcctggtactattgtaaatttgattgtttttctaatttcttttccagatagcttgttataatttttttttttttgaggccaagtcttgctctgttgcccaggctggaatgcagtggcatgatctcagctcactgtagcctccacctcctgggctaaagcaattctcatgcttcagcctcctgagtagctgggacgacaggcacccgccaccacacccggctaattttttatttttagtagagatagggtttcaccatgttggccaggctgatctcaaactcctgacctcaggtgatccaccctcctcagcctcccaaagtgttgggattacaggtgtgagccaccacacctggcaattttaatttttttgaaacaagatctcaatctgttgcccaggatggagagcagtggcacaatcttggctcactgctgtcttgaccttctgggctcaagtgatcttcccacctcagcctcctgagtagctacaggcatgtgccatcacatccagctaatttttgtattttttgtacagacggggttttgccatgttgcccaggcgggtcttgaattcctggactcaagcgattctgctgtcttggcctcccaaagtgctggggttacaggtgtgagccactgcaccaggcctgaaagcttgttgacagtatttagtaacattactgatttttgtaggttcattttatatcctgcaattttgcagaattcatttattagttctaacaacttttttacatttttattttctttaaaattttttaaagtttctaatagagacagggtcttgctatattgcccaggctagtcttgaactcctggccttatgtgatccttccacctctgcctcccaaggtgctgagattacaggcatgagccaccatgtccagccagctctaacaatttttaatgaagtctttagggatttctatatataagactatgtcatctgcaaaaagataattttacttcttcccttccaatttggatgcttttttttttcttgactaattgctttggctaggacttctagtactatgttgaccaagagtggtgagaatggacatcactgccttgttcctgatcttaaagtaaaacctttcagtttttcaccactgagtatgatgttgactgtgggtttttcacatatgaccttgtcctcaaagtcctagcctatcttaatgatctatgaaatgcctttcaggtcattctcccattgtcttgatgtagaagaacacttggctcccttctatccatactaatttcgttatcgaattttactataagtggccaaaagaagccatgcagcaccttgagcactttcctgcttatctatttcttctaccagatagagtagatccttgctcttaagttccatttttcacacagttttagggaacagacacaagtccatcaagttctttgcaactgtgtagcaaggatggcctttactgcagtttccaataccttgtttttcatttgtgtctgaggcctcatcagaattgcctttaccttccacatttctaccaacattctgattatgaccacttaagtaatcgctaggaatttccacactttccctacatttcttcccttcttctgagcccgcatcagaattgcccttaatgctctgttcatggcaatatggacttttttctagcctgttcctccaaactcttccagcctctgcctattatccagtttcaaagctgcttccatatttttaggtacctgttacagcaacacctcacctctctggcaccaattttctatcttcatctgttttttgctgctataacagaatacgtaagactgggtaatttataataaacagaaatttatttggcttatggttctggttgttgagaaatccaacattgaggcattgcatctagcaatggtcttcttgctgcatcatcccatggcagaagggcagaaggccgaaagggcacttgtcaggggtggggaaggaggctatactcatcctttcatcaggaatccacacccacaataactaatccactcctgcaacaacagcattaattcattcacaagggcagagccctcatgaactgatcacctcttagaggttccacccctcaacactcttgcactggggattaaatttccaatgcatgaacttcaggcaatgcattcaaaccatagcaacaccttatagttatgacagtctgttttaagctaatgacaacttaaattgaatcacatataacaactctacactttaggagttggagaccagtctgggcaacaaagaaagcctctatctctaaaaaaaaaaaaatttttttttttttttgagacagtctcgctctgtcatacaggctggagtgcagaggtgtgatctcagctcactgcaacctccacctcctgggtctgagtgattcttgtgcctcagcctcccgaatagctgagatgacaggcacgtgctgtgctgacatgcccagataatttttgtattttttgcggagatggggttttgctatgttggccaggctgatcttgaactcctgacctcaaaccatcggtccaccttggcctccccaaagtgctgggattacaggcatgagccagcacacttggcctctacaaatttttttttaattaaaaacaaaacaaaaaacaggccacatgtggtgacttatgcctgtaatgccagcactttgggatgctgaggcaggaagatcacttgagtccaggagttcaagaccagcctgggcaacatagtgagacctcatctctacaaaaaaaaaaattagctgagtgtggtagtatacacctgtagtctcagctattggcaagaggatcacttgagcccaggagttagaagctgcaatgagctatgattgcatcactgcactccagcttgggtgacagagcagacccccgtatttaaaaacaaaccaacaaacaaaactatttcattgggcttcttggatttgtatttccattatttccattttcttccctagatttgggaagttttcaaccaaccttccttccttccttccttccttccttccttccttccttccctccctccctccctcctttcttcctttcttccttccttccttccttccttccttccttccttccttccttccttccttccttccttccttccttcctttctttctttctttctttctctctctctctctctctctctctctctctctctctctttctttccttttctttctttttgagacagagtcttgctcttttatccaggctggagtgcagtggcgtgatctcagctcactgcaacctctgcttcccaggctcaactgattctcatgcctcagcctcctgagtagctgggattatagccattatttctttgaataaactttctgtagtctgttctttttttcttccacttctggaactctcataatgcatatatggttctggttggtggtgtctcaccattaccttaagttttcttcactcttttaaattattttttccttttgctcttctgattgaattcttttccagtgatctctctctgagttcactgatcctttctctacttggtctagtctgctgctgaacccctccagtgaactttttagttcagttattgtgttcctcagctccatgatttatttatttatttcttttttttttttttttgagacagagtcttgttctgttgcccaggctggagtgtagtggcacaatcttggctcactgcaacctccgcctcctggattcaagtgattctccagcctcagcctcccaagtagctaggattacaggcacatgccaccacgcccagctaatttttgtatttttttcatagaggtggggtttcaccatgttggccaggctgttctagaactcctgacctcaggtgatctgcccgcctcggcctcccagagtgctgggattacaggcatgagtcaccgtgcccagccttcagctccatgatttctgtgtggtcctttaaaatgttttctttcttcttgttgaaattctcactgcattcatgtattgttctcttgacctcagtgagcatctttatggaggtattttaaattgtctgtcaggtaaatcatataacttcatttccttagtgtaagtgaggaagatttgtcttgctcctttgtttgaaacatctttgcctgatctttcattttccttgtctctctgtttggcatctacacattacaaagcagacatctttttcagtcttcacagattggcctcacacaggaacatacccacacctattgttgtgggaatcaggaggacgagagagacctcgggggaaagcaagaggatctttattgaatgcactcagacccagcagacttaacatccaaaaactgggccctgaacaaggacagcacttgacttttatacacacttctaaaagggggtgggctagcttgaagcaggcttacagtggcacaaaagcaaggatatagaggcagaacaattaatccaattgtgacaggttcataactcaggactatacatgaacattgctatgcaacccagatgtctgttatctaggttttgctcaaaagagccttgcactggtttatctcataaccttcaccatggtgcccagacggctgtagttcaggcctgctcaggcttctcacgaccttcgctgtacttcttaggtaaaacagaatacttgaagttcctagttacagagaacaagaatctgtaaactcatatcataaaagaaaggaaaatttgtttttcctctccctatgttgagggagtgctggaagagtctccagagcacatttctttgtgtcctatcttctcagatagtgtttatcaagacttttcctgggtctgggccttgcctgctactgcctttgagatgagtcagcctaatacaggaaagcttatttctttctctttttaattttctttttctttctttctttaatttcccacttcactattagcctggttagagattctggggtcctctaccaactctttctcacttcagggagatgcaggcagctgtgtttttttgtccacctattccatactgagccagagatggggtggtgaagggaactgtggcatctaccagcccaagctgctgtctccattcccctgccaggcagctagactatgccagacctgtcagaactctaggactgatgagacagatataagttttctggggatccccagagaaattgggatgctggatgcatggattaactctttccctccccgggagaaagctgagagctaagatttttcatcttctcactctgtgctgagcagaggggaggattaatgatgcctatctgcccaagccactgcctttattctcccctgggtggtagactgtgccagactcatcagagctccaagactggcaagacagaagccagtcctctggggagccctcgtgaaaaagttggtgttctggacatgtgaaacaacttctctcctcccatgggtggagctaggagctggttgggggggtctcttcctgattgtatgacaccacaccagggtaataatctctgattagacagtgtcccaagtctccctactggctttgttgaatctggttttgtgttctccttgcatgcaggagtctttcaattagtttgatttttcataaagggaatatttctgtaaattgttgtgttagtggaggcaagaagagttcagggcttctgattctgccacattgctgatgtcattcttctggggagatattttcaatcatcatcaatttttcttattttataattttaatgcaagattcatcttaaaaggctgatattcagaatactatacttaatggagatgatttgtagtaatttattgacattatctttattgcttttagataaaatgcaatacccaatttcaattaatgttaacccctgtatagaattgaaaagatccaaaggattattgcacattttctacattccaagtaagttcaaatttttgcttttatagaccaatcaaaggagttaagaaatatcagtgataaatgattgtgtttcaaatatacttgtattcgttatctattgctacgtaacaaattaccacgatctttgtggcttaacacagcacacatttattatctcatcatctttgttgatcagtattctgggcatagcttacctgtgtctgcttcaaggtctcccataaggctacaatcaaggtgtttgccaggctggagtcccttctgaggctcaactaggaaatatatacttctaagctcacgtagctgttgtcagtattcagttgttagtattcagtcccttgctggctgttggccacccgtcagttccttaccacatgggcattttcagtatggctgcttgctttttcaaagccagcgagggggacagtcagctaactaggcaagtcacaatcttatatagcataatcatggaagtaacactccatcatctttgctgtgttctattggttagaagcaagtcactaggctagcccatactactgggagaggattacacaagaacatgtgggtagaaatgggaataacttcagctgtccaacaatcttacaggtatatccttcatcaatcattagctataagtaatattgggtttccattagtcaaagatctgtgtgtcagcaagccaggacttcaatattttttaaagatggtctttctagagaaaaatacagtaataatgggatgacagaaggccatgtgttttgttttgctttgtgttgtgtcttggttttcctctctatgactttgcttgttatcagcttagaaaaaactaacgcaggtggggtgatagcatggggctgtatctcagtctctgtgcagacacaaaactttttcctctcctaccagttaccaaacattgtttattgcctgtaagctctggaatcccagaaaactttagttttaatctttatcatcatcattatcacataatttacatcctagtttagatttggagcttgttttagattaatactttacagagtagttttacatgaataagcttaaacattttcccccgattttagttctctggcttaccagaaaaatgaaaaacaacaacaacaaaatccccaaaactgagaacccaggaatgatagacaacaaacttgtgttttaattttcatgattctagttgttcaacctgtttttttgacactctgtatctgcattcatttattcactaaaaagatgcttagtaaattgtaagtatcatgctaggcactgtgaattcattgataagatattctctctctctctctttttttcttttgagatggagtctctgtctgttgcccaggctggagtacagtggcatgatctcgtcggctcactgcagcctctgcctcccgggttcaatccattctcctgcctcagcctcccgggtagctgggattataggcacccaccaccatgcctggctaatttttgtatttttagtagagaaggggtttcaccatgctgaccaggctggtcttgaactcctaacctcaagtgatccacccgcctaggcctcccaaagtgctgggattacaggcgcgagccaccgtgcccagccaataagacattctcctgatcttcaggaatccatcagtgcaataggagagacagaactgcagatagtatggtataagtgaagtgacagcaacagggcttagtgacagcacagagcaaaggatgattaactctatttccttcactttcctggccaaagataaaataaataatctaaagttttgcttggctaaggctctacagtttcttcttttttcctaggttctgttttctggtctgttggcaggttttacccctacacaattggcataaaggatgatttacataatgaataatctcactttcaagtgaatcccttgcccttactcgcaggaaacaggttgtatctttccaggccaagataataagacagtacgaaatcatttagagttctctgaaaagcagacactaagaaggaattaggctagagatttcttggaggaaacgcttgtgaacagtaaaggggagaggaagccagagtaggctaggagaaaattcagattggggtgcaggtctgatacctttgaaaagagagtagggaggaaggagaattaagtaggataatcctcagaccagaatgcatccctgagacactcttgggctgggccaaataggaagccccagagcagaatgcctgttgtaagagacccatgacaggcgggagtagcctggctctagggtccttgcagtgctggatcattataggagcagcctacgagagggtggcctctggctgagtgctgagatagatgtgaaggtatggcagctgcaggctgtctgccagctactcttctcacagcagctgcttttgaagggaaatttgagggacatacctcaatggccatcacaagtgttttataatttctcactaaatatgacatttatataacaaattatattaattcattaacaaaataattattcagtcatatttttatagcgcaattatgtttccatagaatatactataatgttggtataaagtgtgaggccacattcatggaaatcaaccttatttttatggcctcatttctacttgaattcaggaagagatacaagtacaaacttgaaccaaaaataggttgagaatggaaatgatatcttttgagatgtcatagccttgctagtaaattatttgtatattattaactgattgacaaatcaaactggagcataagcttccttcaaggttatttctggaagttcctggaattgtctttggtaagctaaaaaatagtcctcttttataccacatgacagcctccatgtttcttttctttttttgagatggagtctcactctgtcacccaggctggagtgcagtggcgtgatctcggcgcactgcagcctttgcctcctgggttccaacgattctcctgcctcagcacccctggtagctgggattacaggcgcctgccactgcacccagctaatttttgtatttttagcagagacagggtttcgccatgttggccaggatggtctcgaactcctgacctcaggagatctgcccgccttggcctctcaaagtgctaggattacaggcgtgagccaccgcgcccagcccatgtttcttgtctctcactccaacacccatattgacatccccagcctgctgagccacaaaagtggtgtctcattgttgccatcgttcatgttcttaacgtgaccacagtggtgtccaaggacaaggagtttctcacaacttaaactattgagttgtcaaagcaggacttcctttcctggagttattgaagactggaatcttgctgtgtttcacgaagaaatggaagaaggtcctttgccctgcattgtcactggacaccacagtggtgctgtcttgtgcttctgtgtctccaggaattccttatgaaaatggtctttcaacttctacacacagcactgcttttggagaagttcaagcccaagtccatggagtaggccatgggatcactgtttgaaacagacgccatgatattcttctccacaccctaccctgaaagttgagaagtgaagacaaattaggaaagttggtggtgcttcaggttcagagcaatgagggcctacatgaaggcagcgcggtaatggggatgaaaaggaggttagaaagatttactgggactgggaattctagtgccaacttggtgatgaagaggaggaaatcacttttagatctgttgggtgaggtaactgggcaacatttgagtagagatattgaataaatacatgggatcaatgctcaggagaaaggttgagactaaagttgacttgagaattgtcaccttatgtggtacttggcctgagagagtgtgcagagtgaaaactaaggagaactgaggatagatcctggagaaatccaggagtcgatgaaatagactgaggaagaggagtcagagagattggaggacacctggaagggagtggttgcaccaacaccaaagaaggtttagggaaaggagcaggcagttctgttaagcatagccaagttcaaattaaagttagaaaagttgtcttttgattatggttacaatgagtcatggtgatcttactaggaaaatattttcagtgaggtagtggttgcaaaattgttttctttatgaaaaggattgtgcatcttttttggccaggcatggtggctcatgcctgtaatcccagtactttgggaggctgaggtgggtggatcacgaggtcaggagttcaagaccagcctggccaagatggtgaaaccccatctctactaaaaatacaaaaattagctgggcacggtggtaggcgcctgtaatcccagctactccagcggctgaggcagaagaattgcttgaacctgggcagcggaggttgcagtgagccaagattacgccactgcactccagtctttctcaaaaaaaaaaaaaaaaaaaaaaaaagaagaaaagaaaagaaaagaaagaaaaggattgtgcatctttttcatttgggccaactgaataagttggaagagttgagaaaaatgcattgattactggaatttttcatggtagctggaaatagggagatcatgaggaaattaaaaaataggcatacatttaatttttcatcaaaagattctattttattctcaagaaatctttcatcagaactcctagaaagttgactgtctcactgtccttcctttccttttcttttcttttgaattttctagcagaaccatagaactagacattttatgtctttatttgatagttttctcaatgaagttatgaaggctcttctgtttttaggcagagcatgcatagttttactgtggaacattgtatttgattatgatttactagttgccctctcagaacttcagttttctcatctgcagaagaagagtaataattgacatcttgtagatagttgtgatgattaaatgaaataatgtaagaaaagcacagggccccttttaactatacaataaatgttaattactatgtataaaagaatctactatttgagggcctaatgggattttttcttttaaagggagagatttaaagcaattatatttcaatctctatataactgtcaacaccatgaatcttccaaagcgcaaagaagttatttgccgaggatctgatgacgattactctttttgcagagctctgaagggaggtaagtattcagttcatattacttttagaataggaaataattctttatgaaaatgttatgaaaattaaatacattgaaaatgggaagttccttttctgctaataattctttttccatccaaagtttttactttagcagcttaaataacattttataactatatacttgaactgttaggatgacacttacatgatttgtattattaatatttttcatagtaacccaccagattttctatacagtccttggtttctttagtgacttctgcctttatgttcctccttggaattatttacagaataataacattcagaaattaggacccttacagagatcttgcaccaagggataaaaaaataaaaaataaaaatgaataaaaaaaaggaattaccactttagagctatattccagcttttccatttaccagcaatgtgacttttggcatgttattaatatttaacctttcttggcctcattttcctcagctataaaatgggaaagtaaaacacatcatacatagttttaagaattaaatttgagaatgtatgttaaaaaaaaaagcctagtacagtgttcagaataagaatccttcttttgttatgaattttccagtcctgtcatctttataatgagggaactgatgtttgcagtggcttagtaatgtgtccatggtcatttggagaactagtaacagagcagcatcctaactctaagacatgactctcctactgtgtcatgctttctctttggaaaaaaaaaatcccttagcagattactgtttttttttttaattgtgcatttcttttacacaatctctgtggaattacattgatattttacctgaatcttcaaagtttctctttcttgagacctcagaggtgtcagactcaaatcagcttgtctacttcttctctccctcctcccactttcttccataaacatctattgaacctaatttgagcaaatcgtgatttacgttctggttatctgaagttgaatggtaggatctccttcctcagaaagttactctggggaaaactggattgccatatgcagaagaataaaactggacccctagctctcaccacatacaaaaatcaactcaagatggatgaacaacttaagcataagatctgcaattacaaacatactagaagaaacctaggaaagattcttctggacattggtctaggcaaagaatttatgacaaagatctcaaaagcacaggcaataaagacaaaagtagacaaatgggatttaattacatcaaaaagcttctgcatagcaaaagaaataatcaacggagtgaacagacaacctgtagtatatgagaaaaaattcgcaaactattcatccaacaggggactaatattcagaatgtacaaagaactcaagcaactcaacagcaacaacaaaaacaaataaccccattaaaaagtgggaagagtggctaggcgcagtggctcacgcctgtaatcccagcactttgggataccaaggcaggtggatcatgaggtcaggagttcgagaccagcctggccaacatagtgaaaccccatctctactaaaactacaaaaattagccaggtgtggtggcatgcgcctgtagtcccagctacttgagaggctgaggcaggagaatcgcttgaacccaggaggtggaggttgcagtgaggcgagaccatgccattgcactccagcccaggtgacagagtgagactccatctcaaaaaaaaaaaaaaaaaaagtgggaaaaggacatgaatagacacttatagatacttttcaaaagaacagatagaaatggtcaataggtatattactaatcatcagagaaaagcaaattaaagttgcaatgagatatcatcttaccccagccagaatggctattcttaaaaagacaaaaagtaacagatgttagcaagaatatggagaaaaaagaactcatccatttttggtgggaatgtaaattagtatagtctatggaaagcaatatggagatttatcaaagaactaaaaatagaaccaccattccatctagcaatttcactactaggtatctactcaaaggaaaagaaatcaatatatcaaaaagatacctgcactcatatgtttattgcactagtcacaatagcaaagatatggaatttacctaagtgtccatcaacagacaaatgggtaaataaaatgtggtatatttacacaatggaatactattaagccattaaaaaaatgaaatcatgtatttttcagcaacatggatggaactagaggccattatcttaactgaaacaaaccaggcacagaaagtcaaatattgcatgttcccactcataagtgggtgctaaaaaatgtgtacacatgtactacagagtggaatgatagtgaagagttggaagggtgagggggtggatgatgtaaaattagttaatgggtatatgttacttgggtgatgggcaccctaaaagccctgacttgaccactacacaacctatgcaggtaatgagattgcacatgtacctggtaaatcagtataaataaataaataaaaaagtcactccattctgggaaagacagttataagaacaaaatttataataaaattatgtgtaattttattgtaaattgattataggtatgagccattgccatacaataaaattatatttacatataatttttttgtaaaattattgtaaattttttgtaaatttattgtaaaaaggtgtatgtaaataacatatagttctattagcaatagaactatatgttattggttttaactatataattactaaataactgtggcagaaaaactgggataagttttttttttgagacaagatcttatcttgtggctctgtcacccagattagagtgcagtggtgcaaccatagctcactgcagcctcaaactcctgggctgaagtcatcatctagtcacagcctcctgagtagcaaggactacaggcacagatcaccatgtccagctaatttttttttctttttttttggagtcagaatctcactctgtcacccaggttggagtgcaatggcacaatctcagctcactgcaacctccacttcccgggttcaagtgattctgcctcagcctcccgagtagctgggactacaggcgtgtgtcaccatgcccggctaatttttgtatttttagtagagacaggttttcatcatgttagccaggctggtctcaaactcctgacctcaggccatctgcctgcctcggcctcccaaagtgcctcccaaagtgctgagattacaggtgtgagccaccgcacctggccctaattttaaatttaattttattttaattaaaaaatagagataaggtctcaccatcttgcccaggctggtgttgaactcctgggctcaagtgatcctcctgcctcagcttcccagagtgctgggattacaggtgtgagccattgctcctggacatttttaaattttttgtagagacagggtctcactatgttgcccaggctggtcagacaagccttcaatagagtattttattagggtcttgaatctgagaacatcagagggtgagggggaaaaacattccctggagaggaaacagcatgtacaaacacatctagtcatgaaagagtatggtttatttggagagtagtttttttgtagttggagcagaatgtgcttgtggggaattgctggagatgagcctagaaaagggggcagtgtgagcatgtaactcatgcgcaaaatattggccggtatcctatagtcagtgggcagggatgacccactgagatttccatttttacagataactagcatccatatggcaggaggtgggggtaggctgaggcaggaaacttgggtggaaggaagagcatccaggaggctctgtgagagaccatgagggctgaacgagacggcggccagggagtagcagggaggagatgaaagagagatttttgggagacagaataggcaggatacactgatttaatgtggcaggcaaggattaggtaaaatcaagaccctcttgggctgggtgcctgtaatcctagcactttgggaggctgaggctaggagtttggggccagcctgggcaaggaggtgaaacaccatctctgaaaaaaaaaaaaaattagctgggtgtggtggtaccagcctttagtccagctacttgggactgaggtgggaggatcacttgcacccaggaggtcttggctagctgtgatccaccgctgcactccctgacttaaaaaaaaaaaaagaaaaaaaaaaaagcaccacacttgagggccagtgactggatgtatggaagtgccattatctgagatgatgtgtacaaaagaacaggcaggtttttttttttttttttttaaagggaaacataatgagttctggcttgggcatgttaagctcaaggtaattttggaatattcaaataaacatggccatctggcagctgagtatgtgggcccagaacttaggactttctgatttaagaagcattgacgtatcaagagacccttgaaatttgggagtcgaatttgcaagtaccatacatatctatgtttaaaataattttccttttggttgttataaaagttatacatctttatgatatgatatttagaaaatatggacaagcaattataaagtaaaagttacccataatctcaccagtttacacacacacacacacacacacacacacacacacatatatctgtttctctcaaaaacacatttatgaatcaatggatggatatttcaaagcatcttaaatttaataagcctgggctgggcacggtagctcatgcctgcaatcccagcactttgtgaggccaagggaggtgggctgcttgacatcaggagtttgagaacagcctgggcaacatggtaaaatgccgtctctaccaaaaatacaaaaattagctgggtctggttgtgcatgcctgtggtcccagatacttgagaggctgaggcgggaggatcactggagcttgggcagtggaggctgcagtgagccgtgattgcaccactgcactccagcctgggcaacagagcaagatcctgtttaaaaaaaaattcataagcctgtaagttctgttaccgggattttgctggggctgctgttactaagtaccataaactgaataccttagaaaaacagaaatttatcatgtaacagctctggagtccagaagtctgaaaccaagataacgaggccatgctccctctgtaggagctaggcatggatctgttccaggcctctctcctagcttctggtggcctaaggcatgtcttgccttgtagatggtcatcttcttcctgggtcctttcactgtggtcttccctcctatgtttgtctatctcagtgtccaaatttccccttcttaataaggacacttgcatattggactagggcccaccctaatgacctcattttaacttgattatctgcaaagatcctgtttctaaataaggtcatgttcacaggcactggaggttagggcttcaacatatctttttgggggacacaattcaatgtacaataccatctgaggctcagatcccgccccctgcatagctgccaggaaaactagactggaccatgagtgagctgcatggataccttttgagaagaggagaggagagcctaagaaggggttctgttctccctcgccctgcctcagttccctcttgccttcttcaatgtgggcaaagcttcaagctaacttcctaagaaaagaatggaagggtccgttccaagatggccaaataggagcagctccagtctgcagctcccagcgtgatcgacacagaagacaggtgttttctgcatttccaactgaggtacctggttcatctcactgggactagttggacagtgggtgcagcccacagagggcaagccaaagcagggtggggcattgcctcacccaggaagtgcaaggggtcaggggatttccctttcctagccaaaggaagctgtgacagactgtaccaggaaaattgggacactcccaccttaatactgcacttttccaatggtcttagcaaacagcacaccaggagattatatcctgtgcatggctcagtgggtcccatgcccacagagccttgctcactgctagcacagcagtccaagattgaactgcaaggcagcagcctggctcgggaggggcgtccgccattgctgaggcttgagtaggtaaacaaagaggcctgaaagctcgaactgggtggagcccaccacagctcaacgaggcctgcctgcctctgtagactccacctctgggggcagggtatagctgaacaaaaggcagcagaaacttctgcagacttaaaggtccctgtctgacagctctgaagagagcagtggttctcccagcatggtgtttgagctctgagatcggacagactgtctcctcaagtgggtccctgacccctgtgtagcctaactcggagacacctcctagtaggggcttactgacacctcatacagctggtgcccctctgagatgaagcttccagaggaaggatcaggcagcaatatttgctgatctgcaatatttactgttctgcaatatttactggtctgcagcctccgctggtgatacccaggcaaacagggtctggagtggacctccagcaaactccaacagacctgcatctgagggtcctgactcttagaaggaaagctaacaaacagaaaggaagagcaccaacatcaacaaaaaggacatccacaccaaaaccccatctgtaggtcaccatcatcaaagaccaaaggtaggtaaaaccacaaagatggggagcaaccagagcagaaaagctgaaaattctaaaaaccagagtgtctcttctccaaaggatcgcagctcctcgccagcaacggaacaaagctggatggagaatgactttgatgagttgacagaagaaggctttagaaggtcagtaataacaaacttcgctgagctaaaggaggatgttcgaacccatcgcaaggaagctaaaaaccttgaaaaaggattggatgaatggctaactagaataaacagtgtagagaagaccttaaatgacctgatggagatgaaaagcgtggcacgagaactacatgacacatgcagaagcttcagtagccaattcgatcaagtggaagaaagggtatcagtgattgaagatcaaattagtgaaatgaagcaagaagagaagtttagagaaaaaagagtagtaagaaatgaacaaagcctccaagaaacatgatactatgtgaaaagaccaaatctacatttgattggttacctgaaagtgatggtgagaatggaaccaagctggaaaacactcttcaggatattatccaggagaacttccccaatctagcaaggcaggccaacattcaaattcaggaaatacagagaacaccacaaggtactcctcgagaagagcaaccccaagacagataattgtcagattcaccaaggttgagatgaaggaaaaaatgttaagggcagccagagagaaaggtcaggttatccacaaagggaagcccatcagactaacagtggatctcttggcagaaactctacaagccagaagagagtggtggccaatattcaacattcttaaagacaagaattttcaacccagaatttcatacccagccaaactaagcttcacaagtgaaggagaaataaaatcctttacagacaagcaaatgctgagagattttgccaccaccaggcctgccttacaagagctcctgaaggaagcactaaacatggaaaggaacaactggtaccagccactgcaaaaatatgccaaattgtagcgactattgatgctaggaagaaactgcataaattaacgagcaaaataacaagctaacatcataatgacaggatcaaattcacacataacaatattaaccttaaatataaatgggctaaatgccccaattaaaagacacagactggcaaattggataaagagtcaagacgtatcagtgtgctatattcaggagacccatctcacatgcagagacacacataggctcaaaataaagggatggaggaagatctaccaagcaaatggaaagcaaaaaaaaataaaaaaaataaataaataaacaggggttgcaatcctagtctctgataaaacagactttaaaccaacaaagatcaaaagaaacaaagaagacaattacataaaggtaaagggatcaattcaacaagaagagctaactatcctaaatatatatgcacccaatacaggagcacccagattcataaagcaagtccttagagacctacaaagagacttagactcccacacaataataatgggatactttaacaccccactgtcaatattagacagatcaatgagacagaaggttaacaaggatatccaggacttgaacacagttctgcaccaagcagacctaatagacatctacagaactctccaccccaaatcaacagaatatacattcttctcagcaccacattgcacttattccaaaattgaccacatagttggaagtaaagcactcctcagcaaatgtaaaagaacagaaatcacaacaaactgtctttcagaccacaatgcaatcaaattagaactcaggattaagaaactcactaaaaactacacaactacatggaaactgaacaacctgctcctaagtgactactgggtacataacgaaatgaaggcagaaataaagatattctttgaaaccaatgagaacaaagacataacgtaccagaatctctgggacacatttaaatcagtgtgtagagggaaatttatagcactaaatgcccacaagagaaagcagaaaagatctaaaatcgacaccctaacatcacaattaaaagaactagagaagcaagagcaaacacattgaaaagctagcagaaggaaagaaataactaagatcagagcagaactgaacgagatagagacaaaaaaacccttcaaaaaatcaatgaatccaggagctgattttttgaaaagatcaacaaaatagatagattgctagcaagactaatgaagaaaagagagaagaatcaaatagacgcaataaaaaatgataaaggggatatcaccaccgttcccacagaaatacaaactactatcagagaatactataaacacctctatgcaaataaactagaaaatttagaagaaatggataaattcctggacacatacaccctcccaagactaaaccaggaagaagtcgaatccctgaatagatcaataacaagttctgaaattgaggcaataattaatagcctaccaaccaaaaaaagtccaggaccagacagattcacagccaaattctaccagaggtacaaaaaggagatggtaccatttcttctgaaactattccaatcaatagaaaaggagggaatcctccctaactcattttatgaggccagcatcatcctgataccaaagcctggcagagacacaacaaaaaaagagaattttagaccaatattcctaatgaacatcgatgagaaaatcctcaataaaatactggcaaaccaaatccagcaacacatcaaaaagcttatgcgccaagatcaagttggcttcatccttgggatgcaaggctggttcagcatacacaaatcaatatacgtaattcatcacataaacaaaaccaaagacaaaaatcacacgattatgtcactagatgcagaaaaggccttcgacaaaattcaacagcctttcatgctaaaaactctcaataaactagctattgatggaacgtatctcaaaataataagagctatttatgacaaacccacagccaatatcatactgaatgggcaaaaactgaaagcattccctttgaaaactggcacaagacagggatgcctctctcaccactcctactcaacatagtgttggaagttctggccaggacaataaggcaagggaaagaaataaagggtattcaattaggaaaagaggatgtcaaattgtccctgttttcagatgacatgattgtatatttggaaaaccccatcatctcagccccaaatctccttaagctgataagcagcttcagcaaagtctcaggatataaaataatgtgcaaaaatcacaagcattcctatacaccaataacagacaaacagagagccaaatcatgagtgaactcccattcacaattgctacaaagagaataaaatatctaggaatccaacttacaagggttgtaaaggacctcttcaaggagaattacaaaccactgcccaacgaaataaaagaggacacaaacaaatggaagaacattccatgctcatggatgggaggaatcaatattgtgaaaatggccatactgcccaaggtaatttatagattcaatgccatccccatcaagctaccaatgactttcttcacagaattggagaaaactactttaaagttcatctggaaccaaaaaagagcctgcattgccaagacaatcctaagcaaaaagagcaaagctggaggcatcatgctacctgacttcaaactatactacaaggctacagtaaccaaaacagcatggtactggtaccaaaacagagatatagaccaatggaatagaacagaggcctcagaaataacaccacacatctacaatcatttgatctttgacaaacctgacaaaaacaagcaatggggaaaggattccctatttaataaatggtgctgggaaaactggctagccatatgtagaaagctgaaactggatcctttccttaaatcttatacaaaaattaattcaagatggattaaagacttaaatgttagacctaaaaccataaaagccctataagaaaacctaggcaatatgattcaggacataggcatgggcaaggactttatgactaaaacaccaaaagcaatggcaacaaaagccaagatagacaaatgggatctaattaaactaaagagcttctgcacaacaaaagaaactgtcatcagattgaacaggcgacctacagaatgggagaaaatttttacaatctacccgtctgacaaagggctgatatccagaatctacaaagaacttaaacaaatttacaagaaaaaatcaaacaaccccatcaaaaagtgggcaaaggatatgaacagacacttttcaaaagaagacatttatgcagccaagagacacatgaaaaaatgctcatcatcactggtcatcagagaaatgcaaatcaaaaccacaatgagataccatctcacactggttagaaaggcgattgttaaaaagtcaagaaacaataggtgctgcagaggatgtggagaaataggaatgcttttacactgttggtgggagtgtaaactagttcaaccattgtggaagacagtgtggcaattcctcaaggatctagaactagaaataccatttgacccagccatcccactactgggtatatacccaaagggttataaatcatgctactatgaagacacatgcacacgtatgtttattgcagcactattcacaacagcaaagacttggaaccaacccaaatgtccatcaatgatagacgggattaagaaaatgtggcacatatacaccatggaatactatgcagccataaaaaacgatgagttcatgtcctttgtagggacatggatgaagctggaaaccatcattctgagcaaactattgtaaggacagaaaaccaaacactgcatattctcactcataggtgggaattgaacaatgagaacacttggacacagggcagggaacatcacacaccagggcctgtcatggggtggggggctgggggagggatagcattaggagaaatacctaatgtaaatgactaattaattggtgcagcaaaccaacatggcacatgtatacatatgtaagaaatctgcacattgtgcacatgtaccctagaacttaaagtataatttaaaaaaaaaagaaaagaaaagaatggaaggtatgagcaagatatgagctttcttggcaggcacagtggctcaagcctgtaatcccagcactttgggaggccgaggcgggcagatcacttgaggttaggagttccagaccagcctggcctacatggtgaaacccagtctgtactaaaaatacaaaagtagatgggtgtggtagcacatgtttgtaatccccgctaccacccgggagcctgaggcaggagaatcacttgaatgaaggcagaggttgcggtgagccaagatcccaccactgcactccagcctgggctacacagtgagactccagtctcaaaaaaagaaaaaccaaaaaaaaagatagctttctaagacagacaaaacaattgaaaaacaaaaaaaaaagggttctatccaacagaagaggttagaactccttccacctacctgaactgctaagtgttaggacagtctcccttaatagaaatctagggttttttctttttttctttttttttttttttgtgacagggtctcgccctgtcgcccaggctggagtgcaatggcgcagtctcagctcactgcaacctctgcctcccaggttcaaaagattctcctgccccagcctccccagtagctgggattataggtgcatgccaccacgcccaacaactttttttgtatgtttaatagagatggagtttcaccatgttggccaggctggtctcaaactcctcacctcgtgatcagcccacctcggcctcccaaagtgctgggattacaggcgtgagccactgttcctggtctagtggttgttcttaatttctttctcatatcctcaagtctcatccatcttcctcccctgccccttacacaaagtttacagagttggctttatgaccagagtccagggccccatatacggaagggccccatgcttggggtttaatgccctgtgatcatagtcttgaaattcttaataattttatctttgagtgtgtgttttgtgtaagtgaaatctaatgagatgatgaggtatgaacccaccttccctggaccggttcttggttcccgctgcccacttccctgctggcaccatgggatcccctcccctcttctgtccagtgcaacctgctaccctctgctcctggtgaaatcctggctacagggagggtcaggatccggtgtgggtgccctgcagtatcttgggtgggcatgacagtggctgccctacctgggctggcagcaccacagccctgttcagcagctgactagttgggcacaccatcccctgccccaccccgaaacaggtagtgtgtggaggtattgcatggaggttgcagtgcgttgagagtcactgttgaatagcaggcctgtgggaaagaaaggtacttgcttggacttcctgcctctcacaggagcatggggcatctggcagtttgtggaaggaggacgctgacaggcaactctcaggcccaagcttgcacaggagcccccagtcgtggaggagcccctgggtgtttctgcactcacctatgctgcttgctgaggggtcattccctcaggctgttccatgtcttggggaccttctctttctccgttcaaatttcctgcctctggcttgtgttagtttcctctggccagctcgaggcaccctctggggttgagcagggaaagacaaattatgtaatctccgtgattctaccttgaagttaaatgctttaatatttgcatttaaaactggcattgtattataaaaagatgaatagtgaaattcatactaacagtttaaaatgttctctttattacaaatgatattaaattgcaataaaaacattatggcaagtctagagagagaaacacacagaagaaaggaaaaagttttatgttctagtaactttaacaggtttcccccatcttttgaataagcagcctcatattgtcattttgtgctgggccttgaaaattatgtatttaattatgtaatgatgtattgcattacttttcatggcaaaaactgcaattacttttacaccaacctaatagttgatcctgaagctgggtactgagaattaggtgctaggtgccagatatccaaatatccaaagcaaaaccccggtcctcatggagattatagactagtggagaaaacaagatcctaatgaagtaataaaataaagaaaagcagaaaaatgaaatatgattcaatttgtgataaatactctcaaagaaacaagcaagttaatgagatagagcctattattattatattattatataatatatacatatatgactaatatattaatgcattaatattcacaaggtatgaatatgttattattcacaagatgccattggaaggagggtctagaagggagaaggaataatattaaatgttataagtaacatttaatggtaataatatttaataagtgctatgtcctaggcattgttcttagcacttaacatgtattaactggtatagcccttgtaataatcctatgagatacgtgttatcgcccatcagcattttactgaggcatagaaaaattaagtgactggcctaaagttacacaccaagtagaggagctgggattcaaacccaagccgttctggccccagagccatcactcttacaaagatgttatctttatccttactgcatctttctcattgcatattgtatttggaatggtttcttcttatcattttaggggtctttttttggtttttaaaaattttaattttattttaatttaatgtatttattttgagacaaggtctccctgtcacccaggctggagtgaagtggcacgaacacagctcactgtagcctctacctactgggatcaagtgatcctcccaccctagcctctcgagtaactgggattataggcacatgcaccacacccatctttcttttatatattttgtacagataaggcttcaccatgttgcccaggctggtcttgaacttctgggctcaagggatctaccctctttggcctcccaaagtgttgtgattacaggcatgagccaccgctcacagctggaatggtttcttaaatatctatatctccaatagactttagctctttgagagaagggacagagttgtactaccattgtcttcccaggccctggtgggtagtaggctttcaataagtgagttgttgaactgttaaattttgaattggattggccttgaatgaaagatcataagattatgaagacatgatcactttactgagctcaagaaacacctcaggtatcaactcctttagtaaggatacacccggctaggtgtggtggtttatgtctgtaatcccagcactttggaaggccgaggcaggcagatcacctgaggttaggagttcaataccagcctggccaacatagagaaactctgtctctactaaaaatacaaaaattcgccaggcgtagtggtgggtgtctgtaatcccagctactggggaggctgaggcaggagaatggcgtgaacccaggaggcggagcttgcagtgagccaagattgtgccactgcactccagcctgggcgacagagcgaaactccgtctcaaaaaaaaaaaaaaaaaaaaagatacaccctttatctgctttaaagaaagagcaaaaagaaaagaaaagacagctgggcgcagtggctcatgcctgtaatcccagcactttggaaggccaaggcgggcagatcgcctgaagttgggagttcgagaccagcctggtcagcatgatgaaaccctgtctttactaaaaatacaaaaattaatcgggtgtggtggcaggcgcctgtaatctcagctactcaggaagccgaggcaggagaatctcttgaacccgtgaggcagaggttgcagtgagctgagatcacgccattgtactccggcctgggcaacagagtgagactccatctcaaaacaaagaaacaaacaaaaacagaaagaggatacatccagaagtaaataaataaatgcttacttgtgagactagaagtatacatgcagacattaattatttacttctgagaacaatattatagtttttgatttaactatattaaaaatatttttggccactgtttcttttgtttgagattcaaaacatacatttctatctttaccccaccacattcactactttgttctgactggaacagtaggatgtgcatttttctgcacttttgactctgtgctccacagcttgtgtggggccaggcccgtggctatgcacttaaacagaagctaacttgagcttctcagtggtgtaattgggaggagcttcagtattctggctgcctccaaaatatctcatctgtctttgcaagattgttctctattatacccagcttaatgtgcatcaatatctctgaaaactagtttgcaatgcacatatgtaacttgttctgaagcatattagcactagcttgttcatcgtttattcattattgagaccagcaggtcttaatagtttgtgtcccctaagggataaggtaggggcaggtgtacagagaggctctaggggcggagaaaggaagccacaggagaatagcagccagggtataaaagattcattatttttctacagttttgcttgggactgcgtctgtcagtagttaaggtatactccatgctaccaagaaaaaaatatcacctaaccgtgaccaataacgttttgtattttatattttgtttgcagagactgtgaatacaacaatatcattctccttcaagggaataaaattttctaaggtattgttcaagatttattttgtactgtctaacctttagcagtaatagacatgttaagcatttgaaacaagcaattcacaacttcttcctttttctttcttttttttttttaaatgggatctcagtctgttacccaggctggagtgcagtggcacgatctctgctcactgcaacctccacccgccaacccccaccccaagcaatcctcctgccttagcctcctgactagctgggactacatgtccatgccacaatgccagctaatgttttgtacttttggaagagacgcggtttcaccatgttgcccaggttggtcttgaattcctgagctcaagcgatccactccgcttagcctcccaaagtgctgggattacaagtatgggcagttgtgcccagctgacaatttcatgaatagcagttcgagattaagatgttcagggttttttctttcacttttatttagaattagctttttttttttttttttaactgagtcttgctctgttgcccaggctggagtgaagtgtgttctggactcactgcaacttccgcctcccaggttcaggtgattctgtgcctcaaccacccgagtagctgggattacaggcacatgccactatgcccagctaatttttttatttttagtagagatggggtttcaccatgttgcccaggctggtctcaaactcctggcctcatgtgatccacccacctttgttcctggaccaaactgagggtcgggctgccatttctcgcagcccaatagcaagatgcagatgaactggggaggaagagagcttttatttctgtaactggttacagggagaaggtctggaaattatcgccagaccaactcaaaattacaaagttttcagagcttatatacattctaaggtatatgtctatgtgtaagtgtgcattcatctgaagacacaagtgattaacttcttttaatctataattaaggtctgagtcctgaagaccttcatctggagcctcaggaactttacttaatctaaatgggtctgggtgctggggtgattacctttatcttgtctcctgctaaatcatggaggtttgaggagttccttcagacccccagtaaacttggttgtggaagcctggggagtttcttcggacccacaataaaactcgattgatcctaaatgggtcctattaagaatttcttagttattttgttatggtttaagaaagccttaaaggaagggcctgggcaaaactcctgatgggcttttgttacatcccatcctttgcataagggcgctggctttaaatatttaacttaaccactcagtactgaaacagttgttagtgagacctggccttggcctcccaaactgctgggattataggtgtgagccaccacacccagccttagtattaggtcttttggtattttttctttctgtagactatggaaaaattgcacaaaacatagcatttttttccgcttcatttttttttccctctaacatacatgggcaaaagtaatttgaatgcaagtgctcattactaatctgttgaacagtcttcagatgaaagcactggatgaccattgtgtataagccagtaaatctctataactattcatgtaaatataatgtgaacattgtttaggagaataaaaatcacataaattacagactttttttgttcaagatttttttgggaacttagtctggtcatccatgcctttgtaaaaataattgctgcctcaatgaataataccattgtgattctagatatttgcgtgacatatctagtaatgagattttatgcattttacttatggtgaccattttaaaataaaatttaataatgcatattctttcattattttgttataaactaatatctaatatttcaaaacctcttaggttagtttttaaaaataacataatcttataattgggaaataaatgggctttatgctcatttaattcaaagacatggaaaatttcgtctcattggaaaactataaaatcaatccaatttaaatggctttctgctagtaagagaaagttcttacaaagcctctgaaatagtagcatctaacattttgtattacttgtatttcttatacttcattttagggaaaatacaaatgtgttgttgaagctatttctgggagcccagaagaaatgctcttttgcttggagtttgtcatcctacaccaacctaattcaaattagaataaattgagtatttaaaaaaaaa

SEQ ID NO: 2 - DNA, 642 bp, Homo sapiens
  ref <308>: DATABASE ACCESSION NUMBER: NCBI / NM_015364
  ref <309>: DATABASE ENTRY DATE: 2000-04-28
  ref <313>: RELEVANT RESIDUES IN SEQ ID NO: (1)..(642)
agaaatcatgtgactgatgactaagttaaatcttttctgcttactgaaaaggaagagtctgatgattagttactgatcctctttgcatttgtaaagctttggagatattgaatcatgttaccatttctgtttttttccaccctgttttcttccatatttactgaagctcagaagcagtattgggtctgcaactcatccgatgcaagtatttcatacacctactgtgataaaatgcaatacccaatttcaattaatgttaacccctgtatagaattgaaaggatccaaaggattattgcacattttctacattccaaggagagatttaaagcaattatatttcaatctctatataactgtcaacaccatgaatcttccaaagcgcaaagaagttatttgccgaggatctgatgacgattactctttttgcagagctctgaagggagagactgtgaatacaacaatatcattctccttcaagggaataaaattttctaagggaaaatacaaatgtgttgttgaagctatttctgggagcccagaagaaatgctcttttgcttggagtttgtcatcctacaccaacctaattcaaattagaataaattgagtatttaaaaaaaaaaaaaaaaaaaaaaaaaaaaa

SEQ ID NO: 3 - DNA, 552 bp, Homo sapiens
  ref <308>: DATABASE ACCESSION NUMBER: NCBI/ NM_001195797
  ref <309>: DATABASE ENTRY DATE: 2010-09-29
  ref <313>: RELEVANT RESIDUES IN SEQ ID NO: (1)..(552)
agaaatcatgtgactgatgactaagttaaatcttttctgcttactgaaaaggaagagtctgatgattagttactgatcctctttgcatttgtaaagctttggagatattgaatcatgttaccatttctgtttttttccaccctgttttcttccatatttactgaagctcagaagcagtattgggtctgcaactcatccgatgcaagtatttcatacacctactgtgggagagatttaaagcaattatatttcaatctctatataactgtcaacaccatgaatcttccaaagcgcaaagaagttatttgccgaggatctgatgacgattactctttttgcagagctctgaagggagagactgtgaatacaacaatatcattctccttcaagggaataaaattttctaagggaaaatacaaatgtgttgttgaagctatttctgggagcccagaagaaatgctcttttgcttggagtttgtcatcctacaccaacctaattcaaattagaataaattgagtatttaaaaaaaaaaaaaaaaaaaaaaaaaaaaa

SEQ ID NO: 4 - DNA, 11129 bp, Homo sapiens
  ref <308>: DATABASE ACCESSION NUMBER: NCBI / NG_007086
  ref <309>: DATABASE ENTRY DATE: 2007-11-16
  ref <313>: RELEVANT RESIDUES IN SEQ ID NO: (4980)..(16108)
ggaaaaaaaagatgcgccctctgtcactgagggttgactgactggagagctcaagtgcagcaaagagaagtgtcagagcatgagcgccaagtccagaaccatagggattattggagctcctttctcaaagggacaggtaaggaaaaaagtctttctttgaattcctggaatttagttgaaaattttggacttcaaaatttgtaaggtgttattgtctagttagttcagttttctgatacaatctggccaggaaatgcatattttaaagtcctctcaccattttccaacattgtataattatagtcacatatccacttacttttgtggctcgtgatctcagccaaggctaaattcatttgaacctacagaatgttttctcatattttttaggagaaaatatttttctttgaattgaaatggactctttctgcattgtagctactcattgaggtttagttgctcttgttgtttttgatgttactgtgtgtatgtagaggaagagagaattttaagaaatacatttgtttagttatgtaattaatgcctagaaaaatttaatagtttggtggtaaccttcaggcttcttataatttggataacctaaatattattgtttcatgcaactttgtgaataattcatgttatgttagggttttaatgtttctatgaaactgttcattcatggactaggtagaatcctttatgtgcctcacttggaaggggaaaaaaagaacaaaaaaagaaagaaaatcctactgcgggttggcaactctaaaaggattttaatggatttatgaaacccccagatttccttacagccacgagctgtgaatccacacatgccagcaacattgaatacacttttttttctgcctgggcacacttattctagtaaagagagtgtgatgacagaacaccttagacttcaacacgcatctgtgcttaaagaggataaaaaacaaagaacaaaatcaaacaagacaatgtatgagttgaaactactaaggttgattttctccaggtttctcaggatctgggcagcgttttgcttcctcttaatttggaacccttgctgtgtactctgactttttactgcaaaacaaattgagagaggccagaggttagaggcccaaactgcatttggactgtcagtaaatactactttgcttcccctggaaatcctccaaaagtctctcccaaacacagggacattttgctgacacagaaatgttccttttaaaatccacactgttctcacccactcttgaacaggttataataaatatataatttaaaaacctcacaaaagggaagcaagtgtcctccttctaccaccctcctcccattaaaacatggtcatgagaatggagttcattgtctcatgattgtaaggactaagagagagagattttttacactaagtcatagagtagagaggataaaatggccaacatataagtatttgtcctcacctaaaggaggtacaagtttgacaaataagcaaaaggaaaggattagagggcagatgactaaaatgttgagtctgagaaagggcttcatccctgtctcacctccttgaaaggcttgcaaggcagcgaacaaacaataaggtaggtattatttttaccttacagttgggttaaaaaaaagcccagagaggtttggtaatgtgctcaacgtcacacagccttgactgcataattccagacaagataatctgattccaatgcctatgcccttgatttgcactgatgtctcttcagactattcaactccaaaggcgaccttatacttgtggccatttagggattgggcccagcttagaaagtcggtggagctgacaaagcaatttccaaagtgggcagatagagcaagcaagaggaaagcccaatcatgagaagccatttgaataaaacaaataaaaaagaattctagaaaatttaatggctgtgttatagcaaatatagtgtcaactaaggggtctatgagctttgttctaaaatatgaaagggagaagggtaaaaaagccaaggagttttatcaaactgagtattgagtatctactatatgtcagctctacactacaaaatatagtgccccacatgtaaagcaagctggactgaaatctaacaatgaggaaaagacaaaaacagacacaaaactaaaggcaaaacatgaaccaagcagcaccctattggggttgaaagaaggagaaatcatgtttacttgattggggagaggcaggtgtgtcaagcaaaaagggagtaaagaagcaaaatgtgtcaggcagaggatagggcgggtcccttgctgccagggacaggaatttaacatcaaggatttgcctggcacccagggcaagtcttctgttcctttgaatggggaaaaaatacttgcttactttgatgccacagccccaatcctaaagatagacacacgtgtcttttaacacattgtctcatctgacaacatgtaataatcttctgcatctaaaagctctgttggaaattaaaatcagaaagatacagaaaggaactgtgcctttagacctctaattgttcaggggactgaggaagacttgccccttggaggaagagtgttagtttccctaatgtcaaaacggcaagggtagaacttcccatcatactgttaagcaggccaaaacttgaaattactgggttggtctttgtttgtagttttcagctcccagatgatgactttttctgttgcctgctttctccccaagagctctagcttacttatgtgagtacatgcacgtaggtctctttcatgtcacactctttcgatgcttttgcccaatcgttcccttggtttgcagagtttctccatcactctgtcttgttttcctaactacctactcatgtcgcaagactcagcttaaacagcatttatttcttgcagcctttactgacacaccctgtccccaacagtgttaagggcatcctgtttttgttactaggacattttatatcatcactgaagcagctatgtattgaaactggagttaaatgcccatatctcctatcagcttatgagggactcaaagtcaaaagccttttcattattggatctcatctaccagactgcttttgcacaaactacttgttcactgtgttgataacatggtttgacgggaagaggggatgtaggttttggaatccaaagacctgaatatgaacataggctctgccacttattagctgtgtgccagtctgaaaaccatataacctttgtgattctagttctttctatttggcataaaagtcattcagtctaccttgctgtgaagattaaaagagatgatgtaaattcctggctctgtaggagctcaaaaaatgatagttacagttcaactgattaaataatgaaaagggttcctgctgtgagcatctgatggtcatgataatgtctgaagtactttattttttaattgttcagccacgaggaggggtggaagaaggccctacagtattgagaaaggctggtctgcttgagaaacttaaagaacaaggtaatttttaagttgaaaaatgatcagcctgatttcctccccactctgaaggaaagagcaggcccctgtgaacctggagtgtgtctggaatatttacatcagaattgcggtactggttacaacccgagaaacacatcctacaaaagcagcagggcatggggagaatgctcaggaaacattgctggatgtacatcctcactcagatattttgtctttccagccaaactacagtataagataaattgtgtgtgcaagaacgggggttggggtggatggtaggggagccgggggaacagcaaatggaaggcaaggtgcagcaggaagtatgctactagcaagaggacccctatttgtgaaataaaataaaataaaacagtaataatgataataacaacagcgcctagtgtgggcgcagtgtgtatatatacatacatatacataagcatgcaaatgcaaatttacgtatatttttaaagtatgggctcaattttctgttacctttacatataagattctgataaagtgttggtcaggatgtggagaaactggaaccctcatatatttctgatgggaatgcaaatgatacagccactttgaaagtaggtgggcagtttctcaggtaaacatatatttacattttgacccagcagttctactcctgggtgcagagaagtacaactacaaacattcaagtacaagtctttgtgtggatatgttttcatttctcttgggtaagagatttgtgcttgaatgtttgggggcagccatatttataataaccaaaaaatggaaacagcaaaatgcccatcaactggtagatggttaaacaaaaagtgtcacatccatgcaacgagatagtattcaacaataaaaaggaatggactggtcgggcacggtggctgaggcctgtaatcctatcactttgggaagccaaggtgggtggactgtctgagctcaggagttcaagaccagcctgggcaacacagtgaaaccccttctctagtaaaaatacaaaaaaaaatagctgggcgtggtggcatgagcctgtagtcccagctacttgggaggctgaggcaggagaatggcttgaacccggaggcagaggttgcagtgagctgagattgcgccactgcaccccaacctgggcgacagagcgagactccatctccaaaaaaaaaaaaaaaagaaaaaggaatggaccgcaaacacaggcaaaaacatggtagatctcaaaagcagcatgataagacaagaaagactacatgtatgattccactgatacgacattctggaaagacaaaagcatagcaaaagaaagcagatcagcagttcctggggttgggggcaagaattacctctaaaggggcaggatggaatttttcaaactcattgacccgcacagttaaaattagtgagttttagtgaacaagaattacacttcgtaaatctttaaaaaaaaaaaaaaaagacaaatacagacacccagtagagaacagaatgcttgggtaatggacttgtcccttaactgacaagcataatttggtcacgtgttgtatcaggtgaaatttcttggaggcgttactaaatcagattcacacagacatgtaacaaataccatatttaatggccaattagttcagaacaccgaactgaatcacagttttacatgacactctctacttttcaagcctaattcaaggccttttagggagcacacccagtccttaggaatttggggttacctgcaatttttccttactaaaaagaataactaaaaagctttatttccgaagcttccaaagtgttaggctcagtgctaagtacatattcacatattcttatttatttatgataataccactatgaggcaggttcaatggatactttgtgtgtctgtgtgtgtgtgtatctccagatgaaaatacaccagtgctttctaaattttctgagaatacatatatataccgatatatatgtatttaattattatcaaaatgaagcatgcttactatagaaaggtaaaaagcacataagcagagagagacagagagacagagacagagacagtggcattccctattaagcccaaaccagaagattatcactgttacattttggtgtctagtttaactggatggattcaagaagtgttcactgagagcccagtatacagccgcaccagtgtgtgatactggtaacatgacgtcaagcaaaagcagacatgggtctaccttcccaagatttacagacctttcaggtttttcctttgcttatacttgtgaatatatgcctattttatacaattgagatcatcctacacagactgatttataatctactttttaatttagtaactcaaaactttttaattttagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacaggtcttgttgaataactgtgtctatgggaatctggcacaaaggaagtaaccaaggccataagaagagagaaaatttagaaatatagacagaaaagcattgacctatattttatatcaaattttctgcctttaaaaaaattttataggttacttttattatagaaacagacttcgctcaatttgaagtcttacaatatctgtattttgacctagtacagcagaaaatgtatgaaatatgaatggatttcatctaaatattcatcacaagcttacatttctaaataagtatgtgaggcaattcatagcttatttgttcttaattgccgataatatatacaactcatataggtcaacaatttcaaagcatgtgtttttcattataaaagtaatgatcataacatttttgaaagcatcagacacacacacacaagaaaaaaaatgtggtaattgtacttctctagcaaccatttcaaccagaattgaaaatctggcacatttctttcatggcatcttttttttccttgtacatgatgactttgtttaacgtgtgtgtgtgtgtgtatgtgtgtgtgtgagagagagagagagagagacagatgtctatgttgtgttctcactctacagtgcaaatttccttgttgcttcatgggatgtgcataagcatcataagcatcagttttttaattgctgcatgatttttcatcaaggggttacatcgctatatacgtacatattgcggcatattcaggttgcattataattttcaccattacaaataatgtggcaaagaccatttttgtgcatgagcctctttaaatatttactcccaaggcctaggataattttaccagaaaggcctgattagatctaaaagtagaaacacttctatggctcttgagcagtcaatgacttcaggtctccttggcttcctcttctaaagccaaactgtcaccaaggcctctgtatcaaaactgcaattgggaaactgaggctcgctttctttgttaagtggaaattttaggaatggcacatggcaaaagacaagaaacctaaagggaaaacaaacgtccactgaataatccatacctgaacgttaaggcattattgcttccacttcgagaatctaaaagctcatgattctctgaatgatacttaaacattcacaaatgggagaatacaggcattgtatagtactagcttggtgcaaaagtaattgcagtttttaaatggcaaaaccgcaatacttttgcaccaacctaataaatcctaacttttaacttctgtgcaagatatcatttatgtcttccattccagcaaaatgtctggagcttgaagtatttgggatgaactttgttcctggttgagtgggtaactagatgcattgaatagaatgagtgaacacacgccatacgctggctgaagaacttgagtaagaacatgtaagaacttctgagtgatgctctcaaattataaaagctggattttaagaaaaattgaaagactttgtcataattcacttgagagaattaagtatttaccaagagagatgagtgtacagaaaatgatccaaatttaactctgagaattgaacagtgtctcatgctaaggaatttcttgtgtggctagtatttttatactcatttatttttttggctacgctctaatcgtggttttcttacaaggtggattttgggaaaaaaaatcacagtttttttagtaatgcaatcgatgtaaatataactagttagtgagcattaagtgtaagttatgctttctctattgttttaactaattggcatctccaattcagaacctatcagaaatatcagacactgtgactcaaaggaaaaccaagtgggagcattgagtgaataatatgatgtatgtagtgacactgcaaacctgatgttcacacaaaattttttccccaaaagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaaggtaaaagactggttggtactctagtgcaatagaatactttttagtagacattcaggaggtggaagggaaatgagaaactccatgttatcttattcttggtgtaatctcaaatcattttctctgcagccaataagcaaagggttggttgataaaaggcagtgaggctctctatctctgccttgcaaactgacttaatatatattttctctgctaattcttaaactatccttttagtaggtggggcagagagtaatcatttataaacaggtcagtggaggttcagaggttaaccaacttgcccaagatatatgccaagcctatagtgaagcaagggctagcaccaggtctcctcattccttcctctttccactacatgccacacagttctgcattcttactgagtagtccttctatatcatagaggcttttcaaaaaagaaattttaaaatttcagatataatggtttatgaactaatgtatttactgggagaagcacctatctaaatcataagagaaagaactatctccctgctccctagaaactccttttctaaaagatggaaaataaaatggcagattaagatttaatattacagcttataactttgttgatatgccatagtttgttgatatgccatagtatatcaataaaaagtttttattgatacaattataatatttggtccttatgatgatacaagaggttaaaacctccttatgttaaatgctcagaaataatgtcttttgaactaatgaccaagggatgggtttataaacttgcaaagtatctgcctagtacatgggaaaaactaaacgtcctggaggacaaagcagatatcatcatattatcacattaagaataaccaaaggacaggccacaaatttccctcagctgctctaggccatcctctggattaaggacttactgagaaagggaagtaaagaaggaagctaccaaagtaatgggagcacaatcgtttctttatagaggagccctgggaaagaagggcccagcaggactggggcacaaagtaggtaaatgccaactggttcaaggcaaagtcaaagcaatcaaggggaggcacacatcctcttcttaattgtgtattatttttacatgatttcttctgtatttatattctaaatataagatatacgcaatccaatatgtgtctttacctttgaatgtaggatttgttcaagagaatcatacataaccaagtgaaaacattgtaattttagattcccgatgtgccaggattctcctgggtgactccctgtatatctgccaaggatattgtgtatattggcttgagagacgtggaccctggggaacagtaagcttattccttgatgtgatttgcctccatttttgtccctttgtgtgctagatatgctttactgaccaactctatgagagaaaattaaacatcaagacacacacacacacacatgcccacacacatatatttatatacatgtacatacatatgtatgtatccaggcaattaaatctcaattgacttgctgttaaaaataaatacaaataaaactatattgagttaggttcatagaacctaaatgttttatatattttactatatttatatttcccttaaaaggagacaggcgggcacagtcagccttattaattataattatcttaatttctcttttatagctacattttgaaaactctaggcattaaatacttttcaatgactgaagtggacagactaggaattggcaaggtgatggaagaaacactcagctatctactaggaaggtaggattcttttgtgtgtgcacacatgtgtgtgcaacagaaaaggttgctactgacaaccaaagttattaataaagtctttacatgaaataatgggttgctactttttataaaacaagttaacagattattatctatgaaatgtgaagccatcaaccttaaactgaaatcctttcccacttcttaaaagaaagaaaaggccaattcatctaagttttgatgttgacggactggacccatctttcacaccagctactggcacaccagtcgtgggaggtctgacatacagagaaggtctctacatcacagaagaaatctacaaaacaggtagttaacaatctgaggtaatagagaagcaagtgtacacttgactaatatatatttatacctccttgacctgaaaccaagtcccagctgacactttcagaatgtccatcagtcacatgatgcaataactaaagtgttttccatcggttactacctttttctgttagtggataatctttcaagtctgtctgtactactttcaaaatgtcaactattttataaattacattattacaatttgttgttgtagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagatta

SEQ ID NO: 5 - DNA, 1499 bp, Homo sapiens
  ref <308>: DATABASE ACCESSION NUMBER: NCBI / NM_001244438
  ref <309>: DATABASE ENTRY DATE: 2011-09-16
  ref <313>: RELEVANT RESIDUES IN SEQ ID NO: (1)..(1499)
ggaaaaaaaagatgcgccctctgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagtaactcaaaactttttaattttagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagattaaaa

SEQ ID NO: 6 - DNA, 1475 bp, Homo sapiens
  ref <308>: DATABASE ACCESSION NUMBER: NCBI / NM_000045
  ref <309>: DATABASE ENTRY DATE: 1999-03-24
  ref <313>: RELEVANT RESIDUES IN SEQ ID NO: (1)..(1475)
ggaaaaaaaagatgcgccctctgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagattaaaa

SEQ ID NO: 7 - DNA, 9606 bp, Homo sapiens
  ref <308>: DATABASE ACCESSION NUMBER: NCBI / NG_012050
  ref <309>: DATABASE ENTRY DATE: 2009-05-20
  ref <313>: RELEVANT RESIDUES IN SEQ ID NO: (5001)..(14606)
cgctataaaacccaggccggcaggatcgctgcacccgcggcggcctcctcggtgcgcgacccccggctcagaggactctttgctgtcccgcaagatgcggatgctgctggcgctcctggccctctccgcggcgcggccatcggccagtgcaggtgagctcccgggctccggccccaggtgcccctcggcggtcccctccgtgcccccagctcccgcccctgcagaggatccccccgcgggcgaccggtgagcgtcggtggcgctggggtcccgggttgcgtgtgcgccgggggccgcgagggtgcgggaggactgggcagcgcgcgagcttgtgtgtgcacacgcgtgtcagcaccggctgggggagcgtgtgcacgggtgtgcgcggtgagggtgtgtgcgcgtggcccggcgagacgagttgggagtccctgataaggatgcgtctcaggccgggcgcgggtgctccagcctgaaatgccagcacgttgggaggcagaggcgggtggatcacctgagcccaggagttcgagaccagcatggccaacatggtgaaatcccatctctaccaaaaatacaaacattagccggacgtggtggcacacgcctgtaatcccagctactcgggaggctgaggcccgagaatcgcttgaacccaggaggtggaggttgcagtgagatcacgccactgcactccagcctggtgctccgtttaaaaaaaaaaaaaaaaaaaaaaaaaaaaaaaaaaaaggatgcttctcggacccgcctcaacgcacgccttcttttctcacccagcctcctccgtctgcccgtgggtttccgtccgtcccgctgggactcagcgctgggtgactgccccgcggccagagcaagggcggtctggcagggtcctggaagccgcgcttcccacacgggttccttgcccctgagggtcgcaagctcccctggcctccctaacctccagcgctgaattgggagcctggcgggggcggggggtgggggacccggagccccaaggcctctgcggagccggtctttccggacagggtgattttcgtttaaagattaatggtgaagccgagctcttaattattcctctaggtgggaggtggcaggaaaggcatgttaatatttgataagaagctaaaggcgcttcaagcggctgggcacagggatccgatcctggaggcaccttctttgcaaccccagaccccatcaccggaagaccccctctgccagttctcagcccaaccacccctcggatggttcttggggtccacgcaggctgaaacctcctctcccaagttaggtgctccatcctcaaagcggcggagtgaggggtgaaggtgtcctctgcacagagaggagccagcgtgtgccctttgcagcagggactggctgagctgtcttagagggtcgaggggcaccttgcagcctctccaccgggcccctgggagaagaaagagaaaaagcagaatgtggaaacttttatttgtagggttggacattggaaacctactaagcagctagaaagtagaaagagaaatgctttgcagttctggtgctttgtagctgtgtggtcttgggtaagtcacttcctgtcccatttgcaaaacaggtgttgtcattctgacttcaggggttttaagcgtttgaataagataatgggtttaagtgattagtgcggtgcctgtgtggcacgtggctggtgtgtcatataccaagtttatggggctctcccccaagcctggggccagtgtcaggaatgtgtctcagctcacccctcacacaagctgctatcccctttagtgccaaggctttctgactgggacctgccttccattcatcttatggaggactcaagctgagaagggaccaggttgcatatggcgacccccagagcatctctgtccattgagacaagaggccgggtgaggatatttggtccccttcagagccccagacccctggccgggagcgactgagactttgcccaggaaacacttgggttgtgctgagctgtttttcaggattatcccctgtccctcagagggggaggtggggtgagcaaggtccattagagaggcttcttcagccatcttgaaataagggggaggggtggcaggtcagtcagtgtggggggcagtagctgggtgacaggcgactgactttcggtctctaacctctggtcagggtggatggactaggtaggatgtggcgctggacaccgagacctctgggtaggtagcccctctccttcccaagccctcttgggagtggcctgagtcagtgggctggtacttacctgcaagggtggccctctcattcaacgaagacaggaacctgagtggtgggtgctcctgccaccagcaccccaccctcatccaccgacatggacctgacccctggccttgacctcaccaccattatgttttcctggctgctgcttcctgaagaaggtagttacaagtgcgttcagccaagcaggtcaaaagtctccatgattaagacagtggatcacaggtcaccactacaaacctaggagctgcggccagagccagggaagaccaaattcctaagaaaagctaagcctggaccagatgggccccagggaggctgtgaggcgagttacagggggctccaccctttgcagctggctcttggggatgctggctgaagggaggcttctcctggaatggctgcccccgggccctccagtcagagaagaggccagcccaatcttcaaaaccccttcctccttcaagacctctgcgggtggggacatgggatgattatggcaaggaactgtcatggtgcaaatgccctctgatttattatctccttagtcaccagcactgggaatttggtggctgcacctgtcagaggcgtttgaaccagagtgactccattttgggtgagagctaggaaaatgaggccgagatttgctgggctgcattatcagaaagtcaggcatttctagcctctagatgtttgcagttaagggaacaaattaataatatttactacacagacccagacttgggaaggtccagatactccgatatctggagagcaaaggcattcctaattttgctttaaaaataataatgttggctgggcacggtggctcacacctgtaatcccagcactttgggaggccgaggcgggtggatcatttgaggtcaggagttcgagaccagcctggtcaacatggtgaaaccctgtctctactaaaaatacaaagattagctgggcatggtggtacatgcctgtaatcccagttactcaggaggctgaggcaggagaattgcttgaaactgggaagtggaggttgcagtgagccaagagcccaccactgccctccagcctgggcaacagagcgagactctatctcaataataataataatatcaattcttggataatgtagtaattaagaaaattaatcctttatcacaaacccttgtagcagagcacatgtccccatatatacaacgcattgaatgcattccttccttctctgacttttgggaatgtcctactctgtctatggagtagctgtcctttcaccactgtactttcttaataaacgtgcttttcctttgcactgcagactcaacctgaattctctcttgtgcaagatccaagaaccctctcttggggtctggatcgggacccctttcctgtaacatgccttctgtcagaggaagattatgggactcagaatgtcagataactcccctgtggagactgagcaaaggcagagcgggagctggaggcttcggagctctgccccactgctggggtggcgagtggccactgccctctgcagaatccacccacatgggggtggttcaggtgcagcaactcccagggacagcagagccctgcccggggctggggatgggccatcagggggaggacactgcagctctgaaaaaggcgatgccagcagggaaggagaggcagtgatcagggcagggaaactccaaagcccaaagcagctcttcgtggccagttagttttcctggggatgctccctcatggctccttccccatcctgtccccactccggggactctaccctggcttagactacaagaagatgggactgtgggggcagcccaggcaagaagggagagggtggatgagctcagagacctcagctttggctgagtgggaaccgtgcttttcttaggaatttcctcatcttgatttctttccaagtcacttcatttctctcctctctcggcccctccttggctttggccatctgctcccaaaaggaggccatggggtgggtgggaggggtgggggggagggaaggagttgtcgatgggattttactaagtgccaagccgcctgaaacctcatttaatcctccccaaggccctatgaggtgggtatgtttgtctcatgttacagatgatacaactgaggctcagagagcttaagggacttgcccccaaacccatctaagccccctgagcggctcagccagaattccaagctctggatgagatgttgtgttcgccctgctggtgcagccttttaaaaaaattaatagactttatttttagagcaattttaggctcacataaaaattcagcaggaagtacagagggttcccataggcccttctgccctgcatgcactttcccctcttattaacatcttgcatccatgtgggacatttgaggtgcagcctttttttcagagatgctgggttctattgccctgaggcagagccctcagcctgtccccagagatcaggactgtgcggttgaggggcaggggaggaagcctggctgctttcctcctagcgggagctgtgagacgctagccaagaagggatcttagtaacacagggtgagacattctggggaggggccgcctaatctcctccctcatcttggatctgggaaggctgcccggttgaacctgactctattgtcccaggagcggaggctgggccaagtcctcctcttctcctctcaccagcctcttttccaggtttggggtgggggaggtaagcgcggaggaggctgctggtgaccaaaggatggagtatccaagccagccccatgttagagatgagtggcctcagagggcttccctggagggccagtggctgcttggcctggccctgcctggggaacagagccctatgccagctgacctgttgatcagcccctgcttggttttcaattgagtctctttttctgggccctcaatcctgctgccaggaacactccatcccagcccaagaggggctccaaccccaggggtaggcccagcctctgatcctcctgtgtgtgtgagcaacaagacacacgcacgcacacttcacacccttcctctctgctccagagtcacactggtgctacgaggttcaagccgagtcctccaactacccctgcttgggtgagtacagccagtccaggggactgctctttgtgcatggtgggcaccacgcaagccgaaatggagaccccggaagagtgggaaggggaggggtgatggtggcttcccaggcagatatcagttcccagcatacacacacacacacacacacacacactctctctctctctctcacacacacacacacacagatatacaaacacacacactcacacagaaacacacacacaacacacacacaagcacacactcaaacactcacactcacacatgcacacacacactcacactcacacaaacccacactcactcacacacacaaacacacactcacactctcctgtcctggtgctcagggaccagggagcatagagtgacctgagtcttcactggcacctgctgtcatcgagagggcatgttccctgtggaataaatcggtagcagcattaaaggaagaaaggcgttttgctgtttcccataataaaacggggagttgtttctatcccagtgacaccccttgcagggtgcctgagtgatggtcctttacgcttttatttgaatgctgaccaattcgaatgcaaaccctgcagtctgctgtgttttggtgactgagcagtgctgcccgctcctgaccctggcccacctggtgttggggtcaccagggttcggggaaccagctcctccagggttactcctctccctcccattcctcagatgatgctcagcgcccttcatcagcagcccccccgggggtcccactccacagtttccaaagccctctccatccacctcctttaaactctctgacatcccagggaagcggattatctctgcagttcccattttacatttgggaaacccagggcttggagaggccatgtgcaccacctcatgaagttgtccctaccccggagctcacagtcaacagttggcaaccctgttcccaccctgttccacactggggctagactccccagccttgcatgtatgtttctgtggaggaggaggaggagggcccagggagggcgagagtggagttcggagctgagcatccctgcagcacagccttcaggccaccccaaagcgtttctgtgtgggaactgagtgggtgggcctgacttcagtggggtggtgggggctacaccttggtgccaggcacccgactctcagcccaccttctctccctgctcagtgccagtcaagtggggtggaaactgccagaaggaccgccagtcccccatcaacatcgtcaccaccaaggcaaaggtggacaaaaaactgggacgcttcttcttctctggctacgataagaagcaaacgtggactgtccaaaataacgggcactcaggtgggctggatggaggccccaggcaggcctgggcacccgagttccccaaggactgagaggatggggctcctcccaggagggtgtgccagacccaggcccatctgtgctgtgaggtggctgaaaatcccatgggggaggacagcttccaggaggagagagcactctagtatgttttcgttacttttgtcagaccagtctgggatgtgggggcaggaaacgttccaggaagaaggacatgtgcaaaggcgcggaggcatggaacagctcggcgtgttcagaggactgccaggagctctgtgtggaggaaggacagacagtgaagccaggcagagcccaagcaaggcccaggggcagctgggctggggcccaagggcagatcacccaggcctgaggagtttggacattaccccgaggacaccagggcaccacagaggaggtggacgcagaggaggcaccaggacagagctgcagtttggggcagtggagggtgcaggagagggggaggcaggcagggagaccaaggaggaggccagggaaaggtccggggctgtcccaccctgtcccaccccgcgccacccctgcaggccagaaccagagctcatgaaggttgggaggcaggagacaatgtcccatctgggtgaagctgggatgaagagctagaggaggctgagggaggctggttcgaggactctgccccttctgtgctcccagtgatgatgttgctggagaacaaggccagcatttctggaggaggactgcctgccccataccaggccaaacagttgcacctgcactggtccgacttgccatataagggctcggagcacagcctcgatggggagcactttgccatggaggtgagggccccttcccgactgggaccttgtctgggctctgggcgcgcacctgccttgggcaaggagggtagtccaggcccttcataggtccccttttcacccctccaccccgaccagatgcacatagtacatgagaaagagaaggggacatcgaggaatgtgaaagaggcccaggaccctgaagacgaaattgcggtgctggcctttctggtggaggtgggactcccatcccccacttcccggggaacccggggctgagagcttcttcttaggattcagagacctgggactccagcgaggcaggagggggcggggagactccaacttccgcctctgtttctggggttgcatgtccccgggccaggtggggagcccagagcctcaatcccagaagctgcctggccttccgcccccagatcgggagaatgaactggccaccaccactggctccctgcagactttctcaagacccttccctccctttccaggctggaacccaggtgaacgagggcttccagccactggtggaggcactgtctaatatccccaaacctggtgagtcaggatgggggagaagggcttggggtgaggggggggattcctcccacaaaggaaggggtgggtgtgcggggagctgggctctcagagtgcaggggaagaggggctccttctcccaccctcactgacagtgtcctctgcccctatctcagagatgagcactacgatggcagagagcagcctgttggacctgctccccaaggaggagaaactgaggcactacttccgctacctgggctcactcaccacaccgacctgcgatgagaaggtcgtctggactgtgttccgggagcccattcagcttcacagagaacaggtgcacagggcctggggcagggcatgggctcccactgcctggctccccagaaattatccctctgtctgccctcagaggtccctcaggatacaggtggggagcccaggtaactgaagtccgttgttaatcatcgacattcactgaagacaggcaagaaaagcctgagctgttccatcaccagattgggggctagacaggaggcaggggaaggtggagtcattcagaaaatggtgctggggtttctatcatgaacgaggctctggggagacagcagtgagcccaaaggacccaaatccctacccttgcactgctttcattccaaaacagtggttctcaaagtgcggcccctggaccagtggagtcaacatcatcactgggggaatttgttagaaatgctaatcctcagagctactgaatcaggaactctgggggtggagcccaggcatctggggcccaacaagcccgccccccagctgatgctaacgcatgctcaagtttgagagccacagtcctgtagtaagagtgacaagagaagcaggcattgcgggccccctggggtgtgagtgaaaggaagccaatgggcacttagccttcacccatgccacgcacctcatttacatcccctattcttatcatcttcacgaccaccttgagagccaggggttcagagcccctctttcctaatgagggctcccaggacaggatgaggtgcctgcctgaggtcacacggcagggagtgcagctccccctgccccgacctgctgagccccatcacttccgcagatcctggcattctctcagaagctgtactacgacaaggaacagacagtgagcatgaaggacaatgtcaggcccctgcagcagctggggcagcgcacggtgataaagtccggggccccgggtcggccgctgccctgggccctgcctgccctgctgggccccatgctggcctgcctgctggccggcttcctgcgatgatggctcacttctgcacgcagcctctctgttgcctcagctctccaagttccaggcttccggtccttagccttcccaggtgggactttaggcatgattaaaatatggacatatttttggagaaa

SEQ ID NO: 8 - DNA, 1165 bp, Homo sapiens
  ref <308>: DATABASE ACCESSION NUMBER: NCBI / NM_00717
  ref <309>: DATABASE ENTRY DATE: 1999-03-24
  ref <313>: RELEVANT RESIDUES IN SEQ ID NO: (1)..(1165)
cgctataaaacccaggccggcaggatcgctgcacccgcggcggcctcctcggtgcgcgacccccggctcagaggactctttgctgtcccgcaagatgcggatgctgctggcgctcctggccctctccgcggcgcggccatcggccagtgcagagtcacactggtgctacgaggttcaagccgagtcctccaactacccctgcttggtgccagtcaagtggggtggaaactgccagaaggaccgccagtcccccatcaacatcgtcaccaccaaggcaaaggtggacaaaaaactgggacgcttcttcttctctggctacgataagaagcaaacgtggactgtccaaaataacgggcactcagtgatgatgttgctggagaacaaggccagcatttctggaggaggactgcctgccccataccaggccaaacagttgcacctgcactggtccgacttgccatataagggctcggagcacagcctcgatggggagcactttgccatggagatgcacatagtacatgagaaagagaaggggacatcgaggaatgtgaaagaggcccaggaccctgaagacgaaattgcggtgctggcctttctggtggaggctggaacccaggtgaacgagggcttccagccactggtggaggcactgtctaatatccccaaacctgagatgagcactacgatggcagagagcagcctgttggacctgctccccaaggaggagaaactgaggcactacttccgctacctgggctcactcaccacaccgacctgcgatgagaaggtcgtctggactgtgttccgggagcccattcagcttcacagagaacagatcctggcattctctcagaagctgtactacgacaaggaacagacagtgagcatgaaggacaatgtcaggcccctgcagcagctggggcagcgcacggtgataaagtccggggccccgggtcggccgctgccctgggccctgcctgccctgctgggccccatgctggcctgcctgctggccggcttcctgcgatgatggctcacttctgcacgcagcctctctgttgcctcagctctccaagttccaggcttccggtccttagccttcccaggtgggactttaggcatgattaaaatatggacatatttttggagaaaaaaaaaaaaa